(19) United States
(12) Patent Application Publication
(10) Pub. No.: US 2024/0024368 A1
(43) Pub. Date: Jan. 25, 2024

(54) COMPOSITIONS AND METHODS FOR NEUROPROTECTION AND/OR NEUROREGENERATION

(71) Applicant: The Children's Medical Center Corporation, Boston, MA (US)

(72) Inventors: Zhigang HE, Boston, MA (US); Shane HEGARTY, Boston, MA (US); Feng TIAN, Boston, MA (US); Joanna STANICKA, Boston, MA (US); Songlin ZHOU, Boston, MA (US)

(73) Assignee: The Children's Medical Center Corporation, Boston, MA (US)

(21) Appl. No.: 18/352,730

(22) Filed: Jul. 14, 2023

Related U.S. Application Data
(63) Continuation of application No. PCT/US2022/012311, filed on Jan. 13, 2022.
(60) Provisional application No. 63/138,091, filed on Jan. 15, 2021.

(51) Int. Cl. A61K 35/30; A61P 25/28; C12N 5/0793; C12N 15/11; C12N 9/22; C12N 15/90

(52) U.S. Cl. CPC A61K 35/30; A61P 25/28; C12N 5/0619; C12N 15/11; C12N 9/22; C12N 15/907; C12N 2310/20; C12N 2800/80

(57) ABSTRACT

As described below, the present invention features compositions and methods for neuroprotection and/or neuroregeneration of damaged or degenerating neurons. In various embodiments, the compositions and methods of the present disclosure are used to treat a neurodegenerative disease and/or nervous system injury. The methods in various embodiments include reducing or eliminating activity or expression of a target gene(s) and/or a polypeptide(s) expressed by a target gene(s) in a neuron.

### CROSS-REFERENCE TO RELATED APPLICATIONS

This application is a continuation under 35 U.S.C. § 111(a) of PCT International Patent Application No. PCT/US2022/012311, filed Jan. 13, 2022, designating the United States and published in English, which claims priority to and the benefit of U.S. Provisional Application No. 63/138,091, filed Jan. 15, 2021, the entire contents of each of which are incorporated by reference herein.

### STATEMENT OF RIGHTS TO INVENTIONS MADE UNDER FEDERALLY SPONSORED RESEARCH

This invention was made with government support under Grant No. EY030204 awarded by the National Institutes of Health. The government has certain rights in the invention.

### SEQUENCE LISTING

The present application contains a Sequence Listing which has been submitted electronically in XML format following conversion from the originally filed TXT format.

The content of the electronic XML Sequence Listing, (Date of creation: Jul. 10, 2023; Size: 32,576 bytes; Name: 167705-025002US-Sequence_Listing.xml), and the original TXT format, is herein incorporated by reference in its entirety.

### BACKGROUND OF THE INVENTION

Neurodegenerative disorders afflict millions of people worldwide and can be devastating to the lives of patients and caregivers, as well as causing a huge global economic burden. Neuron degeneration occurs as a result of neuronal injury and neurodegenerative diseases in the mammalian central nervous system (CNS). Neuron degeneration involves both degeneration of the axon, and degeneration of the cell body (soma) and the other neurites of the neuron. In order to treat neurodegenerative diseases and CNS injuries, there is a need to identify methods and compositions to protect the central nervous system (CNS) neuron from such degeneration, and regenerate the neuron if it has already undergone degeneration. Degeneration of neurons actively occurs via a number of pathways and mechanisms, many of which remain to be identified, and inhibiting these mechanisms can protect central nervous system (CNS) neurons from degeneration thereby promoting neuronal survival—known as neuroprotection. Moreover, central nervous system (CNS) neurons typically fail to regenerate when undergoing degeneration. However, degenerating CNS neurons can be induced to regenerate by certain manipulations thereby promoting neuron regrowth—known as neuroregeneration.

Therefore, there is a need for improved methods and compositions for neuroprotection and/or neuroregeneration of central nervous system (CNS) neurons that are damaged or degenerating due to trauma/injury and/or a neurodegenerative disease.

### SUMMARY OF THE INVENTION

As described below, the present invention features compositions and methods for increasing survival or regeneration of damaged neurons. In some embodiments, the neurons are degenerating neurons. In various embodiments, the compositions and methods of the present disclosure are used to treat neurodegeneration that occurs in neurodegenerative diseases and/or after CNS traumas/injuries. The methods in various embodiments include reducing or eliminating activity or expression of a target gene(s) and/or the gene's encoded polypeptide(s) in a neuron.

In one aspect, the invention features a method for increasing survival, or reducing death or degeneration of a damaged or degenerating neuron. The method involves contacting the damaged or degenerating neuron with an agent that reduces the expression or activity of a polypeptide selected from any one or more of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz, thereby increasing survival, or reducing death or degeneration of the damaged or degenerating neuron.

In one aspect, the invention provides a method for treating a damaged or degenerating neuron in a subject. The method involves administering to the subject an agent that reduces the expression or activity of a polypeptide selected from any one or more of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz, thereby treating the neuronal injury.

In one aspect, the invention features a pharmaceutical composition for increasing survival, or reducing death or degeneration of a damaged or degenerating neuron. The composition contains an agent that reduces the expression or activity of a polypeptide selected from any one or more of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz and an excipient.

In embodiments, survival is increased by at least about 10%. In some embodiments, death is reduced by at least about 10%. In some embodiments, protection from degeneration is increased by at least about 10%.

In one aspect, the invention features a method for increasing regeneration of a damaged or degenerating neuron. The method involves contacting the damaged or degenerating neuron with an agent that reduces the expression or activity of a polypeptide selected from any one or more of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930, thereby increasing regeneration of the damaged or degenerating neuron.

In one aspect, the invention features a method for increasing regeneration of a damaged or degenerating neuron in a subject in need thereof. The method involves administering to the subject an agent that reduces the expression or activity of a polypeptide selected from any one or more of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930, thereby increasing regeneration of the damaged or degenerating neuron in the subject.

In one aspect, the invention features a pharmaceutical composition for increasing regeneration of a damaged or degenerating neuron. The composition contains an agent that reduces the expression or activity of a polypeptide selected from any one or more of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930 and a physiologically acceptable excipient.

In one aspect, the invention features a cell contacted with the pharmaceutical composition of any of the above aspects. In embodiments, the cell is a neuron, or a donor- or stem cell-derived neuron.

In one aspect, the invention features a neuron produced by any one of the above aspects.

In one aspect, the invention features a method of treating a neurodegenerative disease or nerve injury in a subject. The method involves, delivering to a subject in need thereof the cell of any of the above aspects, where the cell is capable of differentiating into a neuron.

In one aspect, the invention features a method of treating a neurodegenerative disease or nerve injury in a subject. The method involves delivering to a subject in need thereof the neuron of any of the above aspects, where the neuron exhibits neuronal activity and function.

In one aspect, the invention features a kit containing the agent of any of the above aspects, and instructions for use of the agent in the method of any of the above aspects.

In one aspect, the invention features a cell or neuron suitable for implantation into a subject, where the cell or neuron has been contacted with the composition of any of the above aspects and/or the neuron has been produced by the method of any one of the above aspects. The cell is capable of differentiating into a neuron.

In one aspect, the invention features a method for introducing a neuron into a subject. The method involves administering the cell or neuron of any of the above aspects to the subject.

In any of the above aspects, the damaged or degenerating neuron is contacted with another agent that reduces the expression or activity of an additional distinct polypeptide selected from the group consisting of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930, optionally to achieve a synergistic effect. In any of the above aspects, the damaged or degenerating neuron is contacted with an additional distinct polypeptide selected from the group consisting of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz, optionally to achieve a synergistic effect.

In any of the above aspects, damage to the neuron is associated with an injury or a neurodegenerative disease. In embodiments, the neurodegenerative disease is Alzheimer's disease, Parkinson's disease, Huntington's disease, multiple sclerosis (MS), amyotrophic lateral sclerosis (ALS), glaucoma, metachromatic leuokodystrophy, adrenoleukodystophy, and/or a lysosomal storage disorder. In embodiments, the injury is traumatic brain injury or spinal cord injury, traumatic brain injury, spinal cord injury, spinal cord crush, and/or optic nerve injury.

In any of the above aspects, the neuron is present in a central nervous system (CNS). In any of the above aspects, the neuron is a retinal ganglion cell. In any of the above aspects, the neuron is a mammalian neuron. In any of the above aspects, the neuron is a human neuron, or a donor or stem cell-derived human neuron. In any of the above aspects, the neuron is in vivo or in vitro.

In any of the above aspects, the agent contains a small molecule, polypeptide, or polynucleotide. In embodiments, the small molecule is listed in Table 1. In embodiments, the polynucleotide contains an inhibitory nucleic acid molecule. In embodiments, the inhibitory nucleic acid molecule contains an siRNA, shRNA, or antisense polynucleotide.

In any of the above aspects, the agent contains a CRISPR/Cas system. In any of the above aspects, the agent contains an adeno-associated virus vector containing a nucleotide sequence encoding one or more components of the CRISPR/Cas system. In embodiments, the CRISPR/Cas system contains a guide RNA complementary to at least a portion of the target gene sequence. In any of the above aspects, the gene is disrupted using a CRISPR/Cas system.

In any of the above aspects, the agent is administered locally at a site of injury or is administered systemically.

In any of the above aspects, an axon of the damaged or degenerating neuron grows at least about 250 μm from the site of injury. In any of the above aspects, an axon of the damaged or degenerating neuron grows at least about 500 μm from the site of injury.

In any of the above aspects, the polypeptide is Snrk or Stradα. In any of the above aspects, the polypeptide is Mkk7 or MAPK8IP3.

In any of the above aspects, delivery to the subject is by implantation.

The invention provides compositions and methods for increasing the survival and/or regeneration of damaged or degenerating neurons. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The ACTIVE 688732777v1 following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “5-PRIME-NUCLEOTIDASE, CYTOSOLIC, IA (Nt5c1a) polynucleotide” is meant a polynucleotide that encodes a Nt5c1a polypeptide or a fragment thereof. An exemplary Nt5c1a polynucleotide sequence is provided at NCBI Reference Sequence No: NM_032526.3.

By “5-PRIME-NUCLEOTIDASE, CYTOSOLIC, IA (Nt5c1a) polypeptide” is meant a polypeptide or fragment thereof having at least about 85% amino acid identity to UniProt Accession No. Q9BXI3, and phosphatase activity.

By “ACTIVATING TRANSCRIPTION FACTOR 3 (ATF3) gene” is meant a polynucleotide that encodes an ATF3 polypeptide or a fragment thereof. In one embodiment, an exemplary ATF3 polynucleotide sequence is provided at NCBI Reference Sequence No: XM_011509579.1.

By “ACTIVATING TRANSCRIPTION FACTOR 3 (ATF3) polypeptide” is meant a polypeptide or fragment thereof having at least about 85% amino acid identity to UniProt Accession No. P18847, which binds DNA and/or regulates transcription.

By “ACTIVATING TRANSCRIPTION FACTOR 4 (ATF4) gene” is meant a polynucleotide that encodes an ATF4 polypeptide or a fragment thereof. In one embodiment, an exemplary ATF4 polynucleotide sequence is provided at NCBI Reference Sequence No: NM_001675.4.

By “ACTIVATING TRANSCRIPTION FACTOR 4 (ATF4) polypeptide” is meant a polypeptide or fragment thereof having at least about 85% amino acid identity to UniProt Accession No. P18848, and which binds DNA and regulates transcription.

By “ACTIVATING TRANSCRIPTION FACTOR 7-INTERACTING PROTEIN (ATF7IP) polynucleotide” is meant a polynucleotide that encodes a ATF7IP polypeptide or a fragment thereof. An exemplary ATF7IP polynucleotide sequence is provided at NCBI Reference Sequence No: XM_006719109.3.

By “ACTIVATING TRANSCRIPTION FACTOR 7-INTERACTING PROTEIN (ATF7IP) polypeptide” is meant a polypeptide or fragment thereof having at least about 85% amino acid identity to UniProt Accession No. Q6VMQ6, and having transcription regulation activity.

By “BASONUCLIN 1 (Bnc1) polynucleotide” is meant a polynucleotide that encodes a Bnc1 polypeptide or a fragment thereof having at least about 85% amino acid identity to NCBI Reference Sequence No: NM_001301206.2.

By “BASONUCLIN 1 (Bnc1) polypeptide” is meant a polypeptide or fragment thereof having at least about 85% amino acid identity to UniProt Accession No. Q01954, and which binds an antibody that specifically binds a Bnc1 polypeptide.

By “Calcium responsive transcription factor (Carf) polypeptide” is meant a polypeptide or fragment thereof having at least about 85% amino acid identity to UniProt Accession No. Q8N187, and having transcriptional regulatory activity.

By “Calcium responsive transcription factor (Carf) polynucleotide” is meant a polynucleotide that encodes a Carf polypeptide or a fragment thereof having at least about 85% amino acid identity to NCBI Reference Sequence No: XM_005246858.3.

By “CCAAT/ENHANCER-BINDING PROTEIN, ALPHA (CEBPA) polynucleotide” is meant a polynucleotide that encodes a CEBPA polypeptide or a fragment. An exemplary CEBPA polynucleotide sequence is provided at NCBI Reference Sequence No: NM_001285829.1.

By “CCAAT/ENHANCER-BINDING PROTEIN, ALPHA (CEBPA) polypeptide” is meant a polypeptide or fragment thereof having at least about 85% amino acid identity to UniProt Accession No. P49715, which binds DNA and regulates transcription.

By “CCAAT/ENHANCER-BINDING PROTEIN, BETA (CEBPB) polynucleotide” is meant a polynucleotide that encodes a CEBPB polypeptide or a fragment thereof. In one embodiment, an exemplary CEBPB polynucleotide sequence is provided at NM_001285879.1.

By “CCAAT/ENHANCER-BINDING PROTEIN, BETA (CEBPB) polypeptide” is meant a polypeptide or fragment thereof having at least about 85% amino acid identity to UniProt Accession No. P17676, which binds DNA and regulates transcription.

By “CCAAT/ENHANCER-BINDING PROTEIN, GAMMA (CEBPG) polynucleotide” is meant a polynucleotide that encodes a CEBPG polypeptide or a fragment thereof. An exemplary CEBPG polynucleotide sequence is provided at NCBI Reference Sequence No: XM_024451327.1.

By “CCAAT/ENHANCER-BINDING PROTEIN, GAMMA (CEBPG) polypeptide” is meant a polypeptide or fragment thereof having at least about 85% amino acid identity to UniProt Accession No. P53567, and which binds DNA and/or regulates transcription.

By “CCAAT/ENHANCER-BINDING PROTEIN, ZETA (CEBPZ) polynucleotide” is meant a polynucleotide that encodes a CEBPZ polypeptide or a fragment thereof. An exemplary CEBPZ polynucleotide sequence is provided at NCBI Reference Sequence No: NM_005760.

By “CCAAT/ENHANCER-BINDING PROTEIN, ZETA (CEBPZ) polypeptide” is meant a polypeptide or fragment thereof having at least about 85% amino acid identity to UniProt Accession No. Q03701, and which binds DNA and regulates transcription.

By “CCCTC-BINDING FACTOR (Ctcf) polynucleotide” is meant a polynucleotide that encodes a Ctcf polypeptide or a fragment thereof having at least about 85% amino acid identity to NCBI Reference Sequence No: NM_001363916.1.

By “CCCTC-BINDING FACTOR (Ctcf) polypeptide” is meant a polypeptide or fragment thereof having at least about 85% amino acid identity to UniProt Accession No. P49711, and having DNA binding and/or transcriptional regulatory activity.

By “CHOP/DDIT3 polynucleotide” is meant a polynucleotide that encodes a CHOP/DDIT3 polypeptide or a fragment thereof. An exemplary CHOP/DDIT3 polynucleotide sequence is provided at NCBI Reference Sequence No: NM_001195054.1.

By “CHOP/DDIT3 polypeptide” is meant a polypeptide or fragment thereof having at least about 85% amino acid identity to UniProt Accession No. P35638, and which binds an antibody that specifically binds a CHOP/DDIT3 polypeptide.

By “CYCLIN-DEPENDENT KINASE 9 (CDK9) polynucleotide” is meant a polynucleotide that encodes a CDK9 polypeptide or a fragment thereof having at least about 85% amino acid identity to NCBI Reference Sequence No: NM_001261.4.

By “CYCLIN-DEPENDENT KINASE 9 (CDK9) polypeptide” is meant a polypeptide or fragment thereof having at least about 85% amino acid identity to UniProt Accession No. P50750, and having kinase activity.

By “DEATH INDUCER-OBLITERATOR 1 (Dido1) polynucleotide” is meant a polynucleotide that encodes a Dido1 polypeptide or a fragment thereof having at least about 85% amino acid identity to NCBI Reference Sequence No: XM_011528507.1.

By “DEATH INDUCER-OBLITERATOR 1 (Dido1) polypeptide” is meant a polypeptide or fragment thereof having at least about 85% amino acid identity to UniProt Accession No. Q9BTC0, and which binds an antibody that specifically binds a Dido1 polypeptide.

By “degenerating neuron” is meant a nerve cell that is undergoing a progressive loss of structure and/or function, including neurite degeneration and cell death. In various embodiments the degenerating neuron is undergoing retrograde degeneration. In various embodiments, degeneration of a neuron involves loss of functional activity of the neuron and/ordegeneration of the neuron's axons and dendrites. In some embodiments, degeneration of a neuron is associated with a neurodegenerative disease and/or nerve cell injury.

By “E1A-BINDING PROTEIN, 300-KD (Ep300) polynucleotide” is meant a polynucleotide that encodes a Ep300 polypeptide or a fragment thereof having at least about 85% amino acid identity to NCBI Reference Sequence No: NM_001362843.2.

By “E1A-BINDING PROTEIN, 300-KD (Ep300) polypeptide” is meant a polypeptide or fragment thereof having at least about 85% amino acid identity to UniProt Accession No. Q09472, and having transcriptional regulatory activity.

By “EARLY B-CELL FACTOR 3 (EBF3) polynucleotide” is meant a polynucleotide that encodes a EBF3 polypeptide or a fragment thereof. An exemplary EBF3 polynucleotide sequence is provided at NCBI Reference Sequence No: XM_017016027.1.

By “EARLY B-CELL FACTOR 3 (EBF3) polypeptide” is meant a polypeptide or fragment thereof having at least about 85% amino acid identity to UniProt Accession No. Q9H4W6, and which binds DNA and regulates transcription.

By “ELM2 and Myb/SANT-like domain containing 1 (ELMSAN1) polynucleotide” is meant a polynucleotide that encodes a ELMSAN1 polypeptide or a fragment thereof. An exemplary ELMSAN1 polynucleotide sequence is provided at NCBI Reference Sequence No: NM_001043318.

By “ELM2 and Myb/SANT-like domain containing 1 (ELMSAN1) polypeptide” is meant a polypeptide or fragment thereof having at least about 85% amino acid identity to UniProt Accession No. Q6PJG2, and which binds an antibody that specifically binds a ELMSAN1 polypeptide.

By “enolase-phosphatase 1 (Enoph1) polynucleotide” is meant a polynucleotide that encodes a Enoph1 polypeptide or a fragment thereof. An exemplary Enoph1 polynucleotide sequence is provided at NCBI Reference Sequence No: XM_005263168.1.

By “enolase-phosphatase 1 (Enoph1) polypeptide” is meant a polypeptide or fragment thereof having at least about 85% amino acid identity to UniProt Accession No. Q9UHY7, and having phosphatase activity.

By “FAST KINASE DOMAINS 5 (Fastkd5) polynucleotide” is meant a polynucleotide that encodes a Fastkd5 polypeptide or a fragment thereof. An exemplary Fastkd5 polynucleotide sequence is provided at NCBI Reference Sequence No: NM_021826.5.

By “FAST KINASE DOMAINS 5 (Fastkd5) polypeptide” is meant a polypeptide or fragment thereof having at least about 85% amino acid identity to GenBank Accession No. BAB47421.2, and which binds an antibody that specifically binds a Fastkd5 polypeptide.

By “Fgfr3 polynucleotide” is meant a polynucleotide that encodes a Fgfr3 polypeptide or a fragment thereof. An exemplary FGFR3 polynucleotide sequence is provided at NCBI Reference Sequence No: XM_011513420.1.

By “Fgfr3 polypeptide” is meant a polypeptide or fragment thereof having at least about 85% amino acid identity to UniProt Accession No. P22607, and having tyrosine kinase activity.

By “FIBROBLAST GROWTH FACTOR RECEPTOR 2 (Fgfr2) polynucleotide” is meant a polynucleotide that encodes a Fgfr2 polypeptide or a fragment thereof. An exemplary FGFR2 polynucleotide sequence is provided at NCBI Reference Sequence No: NM_001320658.2.

By “FIBROBLAST GROWTH FACTOR RECEPTOR 2 (Fgfr2) polypeptide” is meant a polypeptide or fragment thereof having at least about 85% amino acid identity to UniProt Accession No. P21802, and having tyrosine kinase activity.

By “Flt1 polynucleotide” is meant a polynucleotide that encodes a Flt1 polypeptide or a fragment thereof. An exemplary F161 polynucleotide sequence is provided at NCBI Reference Sequence No: XM_011535014.1.

By “Flt1 polypeptide” is meant a polypeptide or fragment thereof having at least about 85% amino acid identity to UniProt Accession No. P17948, and having tyrosine kinase activity.

By “Flt4 polynucleotide” is meant a polynucleotide that encodes a Flt4 polypeptide or a fragment thereof. An exemplary Flt4 polynucleotide sequence is provided at NCBI Reference Sequence No: XM_017009265.1.

By “Flt4 polypeptide” is meant a polypeptide or fragment thereof having at least about 85% amino acid identity to UniProt Accession No. P35916, and having tyrosine kinase activity.

By “FORKHEAD BOX Q1 (Foxq1) polynucleotide” is meant a polynucleotide that encodes a Foxq1 polypeptide or a fragment thereof having at least about 85% amino acid identity to NCBI Reference Sequence No: NM_033260.4.

By “FORKHEAD BOX Q1 (Foxq1) polypeptide” is meant a polypeptide or fragment thereof having at least about 85% amino acid identity to UniProt Accession No. Q9C009, and having DNA binding activity.

By “Frk polynucleotide” is meant a polynucleotide that encodes a Frk polypeptide or a fragment thereof. An exemplary Frk polynucleotide sequence is provided at NCBI Reference Sequence No: XM_005266881.2.

By “Frk polypeptide” is meant a polypeptide or fragment thereof having at least about 85% amino acid identity to UniProt Accession No. P42685, and having tyrosine kinase activity.

By “FRUCTOSE-1,6-BISPHOSPHATASE 1 (Fbp1) polynucleotide” is meant a polynucleotide that encodes a Fbp1 polypeptide or a fragment thereof. An exemplary Fbp1 polynucleotide sequence is provided at NCBI Reference Sequence No: NM_000507.4.

By “FRUCTOSE-1,6-BISPHOSPHATASE 1 (Fbp1) polypeptide” is meant a polypeptide or fragment thereof having at least about 85% amino acid identity to UniProt Accession No. P09467, and having phosphatase activity.

By “FRUCTOSE-1,6-BISPHOSPHATASE 2 (Fbp2) polynucleotide” is meant a polynucleotide that encodes a Fbp2 polypeptide or a fragment thereof. An exemplary Fbp2 polynucleotide sequence is provided at NCBI Reference Sequence No: NM_003837.4.

By “FRUCTOSE-1,6-BISPHOSPHATASE 2 (Fbp2) polypeptide” is meant a polypeptide or fragment thereof having at least about 85% amino acid identity to UniProt Accession No. 000757, and having phosphatase activity.

By “glycerol kinase 5 (Gk5) polynucleotide” is meant a polynucleotide that encodes a Gk5 polypeptide or a fragment thereof. An exemplary Gk5 polynucleotide sequence is provided at NCBI Reference Sequence No: NM_152776.

By “glycerol kinase 5 (Gk5) polypeptide” is meant a polypeptide or fragment thereof having at least about 85% amino acid identity to UniProt Accession No. Q6ZS86, and having kinase activity.

By “LARGE TUMOR SUPPRESSOR KINASE 1 (Lats1) polynucleotide” is meant a polynucleotide that encodes a Lats1 polypeptide or a fragment thereof. An exemplary Lats1 polynucleotide sequence is provided at NCBI Reference Sequence No: XM_017011477.1.

By “LARGE TUMOR SUPPRESSOR KINASE 1 (Lats1) polypeptide” is meant a polypeptide or fragment thereof having at least about 85% amino acid identity to UniProt Accession No. O95835, and having kinase activity.

By “LEUKOCYTE TYROSINE KINASE (Ltk) polynucleotide” is meant a polynucleotide that encodes a Ltk polypeptide or a fragment thereof. An exemplary Ltk sequence is provided at NCBI Reference Sequence No: XM_017022183.1.

By “LEUKOCYTE TYROSINE KINASE (Ltk) polypeptide” is meant a polypeptide or fragment thereof having at least about 85% amino acid identity to UniProt Accession No. P29376, and having tyrosine kinase activity.

By “LIM HOMEOBOX GENE 2 (Lhx2) polynucleotide” is meant a polynucleotide that encodes a Lhx2 polypeptide or a fragment thereof having at least about 85% amino acid identity to NCBI Reference Sequence No: XM_006717323.3.

By “LIM HOMEOBOX GENE 2 (Lhx2) polypeptide” is meant a polypeptide or fragment thereof having at least about 85% amino acid identity to UniProt Accession No. P50458, and which binds an antibody that specifically binds a Lhx2 polypeptide.

By “LIM HOMEOBOX GENE 6 (Lhx6) polynucleotide” is meant a polynucleotide that encodes a Lhx6 polypeptide or a fragment thereof having at least about 85% amino acid identity to NCBI Reference Sequence No: XM_011518521.2.

By “LIM HOMEOBOX GENE 6 (Lhx6) polypeptide” is meant a polypeptide or fragment thereof having at least about 85% amino acid identity to UniProt Accession No. Q9UPM6, and having transcription regulatory activity.

By “LIPIN 2 (Lpin2) polynucleotide” is meant a polynucleotide that encodes a Lpin2 polypeptide or a fragment thereof. An exemplary Lpin2 sequence is provided at NCBI Reference Sequence No: NM_014646.2.

By “LIPIN 2 (Lpin2) polypeptide” is meant a polypeptide or fragment thereof having at least about 85% amino acid identity to UniProt Accession No. Q92539, and having phosphatase activity.

By “LOW DENSITY LIPOPROTEIN RECEPTOR-RELATED PROTEIN 2 (Lrp2) polynucleotide” is meant a polynucleotide that encodes a Lrp2 polypeptide or a fragment thereof. An exemplary Lrp2 sequence is provided at NCBI Reference Sequence No: XM_011511183.3.

By “LOW DENSITY LIPOPROTEIN RECEPTOR-RELATED PROTEIN 2 (Lrp2) polypeptide” is meant a polypeptide or fragment thereof having at least about 85% amino acid identity to UniProt Accession No. P98164, and which binds an antibody that specifically binds a Lrp2 polypeptide.

By “MITOGEN-ACTIVATED PROTEIN KINASE 8-INTERACTING PROTEIN 3 (MAPK8IP3) polynucleotide” is meant a polynucleotide that encodes a MAPK8IP3 polypeptide or a fragment thereof. An exemplary MAPK8IP3 polynucleotide sequence is provided at NCBI Reference Sequence No: XM_024450203.1. An exemplary MAPK8IP3 polynucleotide sequence is provided below:

(SEQ ID NO: 1)
AGTGACGGGCGCAGCCTCGGCAGCGGCGGCGGCGGAGCCCTGAGGCGACAGCAGC
TGCGGGAGGCGACGGGCTGCGGCCTGCGGAACCTGAGGCAGCTGGGGAGGGCCGG
GCGCGCCGGCCGGATAGCGAGCCGCGCTGGCGGCGGCGGTGGCCGCGATGATGGAG
ATCCAGATGGACGAGGGCGGCGGCGTGGTGGTGTACCAGGACGACTACTGCTCCGG
CTCGGTGATGTCGGAGCGGGTGTCGGGCCTGGCGGGCTCCATCTACCGCGAGTTCGA
GCGCCTCATCCACTGCTACGACGAGGAGGTGGTCAAGGAGCTCATGCCGCTGGTGG
TGAACGTGCTGGAGAACCTAGACTCGGTGCTCAGCGAGAACCAGGAGCACGAGGTG
GAGCTGGAGCTGCTGCGCGAGGACAACGAGCAGCTGCTCACCCAGTACGAGCGTGA
GAAGGCGCTGCGCAGGCAGGCGGAGGAGAAATTCATTGAGTTTGAAGATGCTCTGG
AACAAGAGAAGAAAGAGCTGCAAATCCAGGTGGAGCACTACGAGTTCCAGACGCG
CCAGCTGGAGCTGAAGGCCAAGAACTATGCCGATCAGATTTCCCGGTTGGAGGAGC
GGGAGTCGGAGATGAAGAAGGAGTACAATGCCCTGCACCAGCGGCACACAGAGAT
GATACAGACCTACGTGGAGCACATTGAGAGGTCCAAGATGCAGCAGGTCGGAGGAA
ACAGCCAGACCGAGAGCAGCCTGCCGGGGCGGAGCAGGAAGGAGCGCCCCACCTC
CCTGAACGTGTTCCCCCTGGCTGACGGCACGGTACGTGCACAGATCGGGGGCAAGC
TCGTGCCTGCGGGGGACCACTGGCACCTGAGTGACCTCGGCCAGCTGCAGTCCAGCT
CCAGCTACCAGTGTCCACAGGATGAAATGTCCGAGTCAGGCCAGTCCTCGGCGGCC
GCCACACCCAGCACCACAGGCACCAAGTCCAACACGCCCACATCCTCCGTGCCCTC
GGCCGCCGTCACACCCCTCAACGAGAGCCTGCAGCCCCTGGGGGACTATGGCGTGG
GCTCCAAGAACAGCAAGCGTGCCCGGGAGAAGCGCGACAGCCGCAACATGGAAGT
ACAGGTCACCCAGGAGATGCGCAACGTCAGTATAGGCATGGGCAGCAGTGACGAGT
GGTCTGATGTTCAAGACATTATTGACTCCACGCCAGAGCTGGACATGTGTCCAGAGA
CCCGCCTGGACCGCACAGGAAGCAGCCCAACCCAGGGCATCGTGAACAAAGCTTTC
GGCATCAACACCGACTCCCTGTACCATGAGCTGTCGACGGCAGGGTCTGAGGTCATC
GGGGATGTGGACGAAGGGGCCGACCTCCTAGGGGAGTTCTCAGTGCGCGATGATTT
CTTTGGAATGGGCAAAGAAGTGGGGAATCTGCTACTGGAAAACTCACAGCTTCTGG
AAACCAAAAACGCCTTGAATGTGGTGAAGAATGACCTGATTGCCAAGGTCGACCAG
CTGTCCGGGGAGCAGGAGGTGCTGAGGGGCGAGTTGGAGGCTGCTAAGCAGGCCAA
AGTCAAGCTGGAAAACCGTATCAAGGAGCTGGAAGAGGAACTGAAAAGAGTGAAG
TCCGAGGCCATCATCGCCCGCCGTGAACCCAAAGAAGAGGCGGAGGATGTAAGCAG
CTATCTCTGTACAGAATCGGACAAAATCCCCATGGCCCAGCGCCGCCGCTTCACGCG
GGTGGAGATGGCCCGTGTGCTCATGGAGCGGAACCAGTACAAGGAGCGGCTGATGG
AGCTGCAGGAGGCTGTGCGGTGGACTGAGATGATCAGAGCGTCCCGAGAGCACCCA
TCCGTCCAGGAGAAGAAGAAGTCGACCATCTGGCAGTTCTTCAGCCGCCTCTTCAGC
TCTTCCTCCAGCCCCCCTCCGGCCAAGCGCCCCTATCCCTCGGTGAACATCCACTAC
AAGTCACCCACCACTGCCGGCTTCAGCCAGCGCCGCAACCATGCCATGTGCCCGATC
TCGGCAGGCAGCCGGCCCCTGGAATTCTTCCCTGACGACGACTGCACGTCCTCCGCC
CGTCGAGAGCAGAAGCGCGAGCAGTACCGCCAGGTGCGTGAGCACGTGCGTAACGA
CGACGGCCGTCTGCAGGCCTGCGGCTGGAGCCTGCCCGCCAAGTACAAGCAGCTGA
GTCCCAACGGGGGCCAGGAGGACACGCGGATGAAGAACGTGCCGGTGCCGGTGTAC
TGCCGCCCTCTGGTGGAGAAGGACCCCACCATGAAGCTGTGGTGTGCCGCGGGCGT
CAACCTGAGCGGGTGGAGGCCCAATGAGGACGACGCTGGGAATGGAGTCAAGCCA
GCGCCAGGCCGCGATCCCCTGACCTGCGACCGCGAAGGAGACGGCGAGCCCAAGAG
CGCCCACACGTCTCCCGAGAAGAAGAAGGCCAAGGAGCTCCCTGAAATGGACGCCA
CCTCCAGCCGGGTGTGGATCCTGACCAGCACCCTGACCACCAGCAAGGTGGTGATC
ATCGACGCCAACCAGCCGGGCACGGTGGTGGACCAGTTCACCGTCTGCAACGCGCA
CGTGCTGTGCATCTCCAGCATCCCCGCGGCCAGCGACAGCGACTACCCTCCCGGGGA
GATGTTCCTGGACAGCGACGTGAACCCAGAGGACCCGGGCGCAGATGGCGTGCTGG
CCGGTATCACCCTGGTGGGCTGTGCCACCCGCTGCAACGTGCCGCGGAGCAACTGCT
CCTCCCGAGGGGACACCCCAGTGCTAGACAAGGGGCAGGGGGAGGTGGCCACCATC
GCCAACGGGAAGGTCAACCCGTCCCAGTCCACAGAGGAGGCCACAGAGGCCACGG
AGGTGCCAGACCCTGGGCCCAGCGAGCCAGAGACAGCCACATTGCGGCCCGGGCCT
CTCACAGAGCACGTCTTCACTGACCCAGCCCCGACCCCGTCCTCTGGCCCCCAGCCT
GGCAGCGAGAACGGGCCAGAGCCTGACAGCAGCAGCACACGGCCAGAGCCAGAGC
CCAGCGGGGACCCCACGGGAGCAGGCAGCAGTGCTGCACCCACCATGTGGCTGGGA
GCCCAGAACGGCTGGCTCTATGTGCACTCGGCTGTGGCCAACTGGAAGAAGTGCCT
GCACTCCATCAAGCTGAAGGATTCTGTGCTGAGCCTGGTGCATGTCAAAGGCCGTGT
GCTGGTGGCTCTGGCGGACGGGACCCTGGCCATCTTCCACCGTGGTGAAGATGGCC
AGTGGGATCTGAGCAACTATCACCTAATGGACCTGGGCCACCCGCACCACTCCATCC
GCTGCATGGCTGTTGTGTACGACCGCGTGTGGTGTGGCTACAAGAACAAGGTGCAC
GTCATCCAGCCCAAGACCATGCAGATAGAGAAGTCATTTGACGCCCACCCGCGGCG
GGAGAGCCAGGTGCGGCAGCTGGCGTGGATCGGCGATGGCGTATGGGTGTCCATCC
GCCTGGACTCCACCCTGAGGCTCTACCATGCACACACGCACCAGCATCTACAGGAC
GTGGACATTGAGCCCTACGTCAGCAAGATGCTAGGCACTGGCAAGCTGGGTTTCTCC
TTCGTACGCATCACGGCCCTGCTTGTCGCGGGCAGCCGGCTCTGGGTGGGCACCGGC
AACGGAGTGGTCATCTCCATCCCCCTGACAGAGAGTGAGTGGCCTGCACACCTGCA
GGGGCAGTGGTGCTGCCAGAGGTGTACGTGGGTTCACGGGGTGGCTCTGCAGGGCC
ACCTTGGAGGGTGCCTTGCTGCCCCTACGCTGACCGCTCTCCTCTTCTCCCATGCTCC
TCCCATGTCCCCAGCTGTGGTCCTGCACCGAGGCCAGCTCCTGGGGCTCCGAGCCAA
TAAGACATCCCCCACCTCTGGGGAGGGCGCCCGTCCCGGGGGCATCATCCACGTGT
ATGGCGATGACAGCAGTGACAGGGCGGCCAGCAGCTTCATCCCCTACTGCTCCATG
GCCCAGGCCCAGCTATGCTTCCATGGGCACCGCGATGCCGTGAAGTTCTTTGTCTCG
GTGCCAGGGAACGTGCTGGCCACCCTGAATGGGAGTGTGCTGGACAGCCCAGCCGA
GGGCCCTGGGCCAGCTGCCCCTGCCTCGGAGGTCGAGGGCCAGAAGCTGCGGAACG
TGCTGGTGCTGAGCGGCGGGGAGGGCTACATCGACTTCCGCATTGGAGACGGAGAG
GACGACGAGACGGAGGAGGGCGCAGGGGACATGAGCCAGGTGAAGCCCGTGCTGT
CCAAGGCAGAGCGCAGTCACATCATCGTGTGGCAGGTGTCCTACACCCCCGAGTGA
AGCTGCTGCCCTGCCTGGCCCGACCTGTACATAGGACCCCCGACCACCTGACCCCCG
CCCGGCCCGCGGGGTAGCCAGCCAGGCGCCGCCGCCCCTCTTCTAACCTCTCAACCT
GCAGCTTTCACCTGAGTCTGGCCCCTCCAGCGGGCAGGGAGTGCGGGGATGCGGAT
CAGCTGGGAGGAGGAGGGGAGGGGAACTTCCACCCGAGGGGAAGATGCTCTCGGG
ACAGTTTCCCGGGCAGCTCCTGGCCAGCTTCCAGCCCAGAGTCCTCAAGTCCAGGGC
ACCTTGGGCCCAGCGCAGGCAGAATCCGAGGTGGTCCTGGCTCTACCCTGGGCCTCC
TACTCCCCAGCACCCCTGGAGGAGGCAGGGGCTCCCCGCCGCCGAGGCTGCCTGCC
CTGGGCCCACCTCTGCATGCTGCTCATGGGGCCACCCTGCCTCCTGGGCCCTCACTC
TGCCTAGGGGAGCTGGGCCAGGCACTAGCCTTTGCCCAGGGAGGTGGGCCTCAGGC
TGCCCAGGTGCCTGCACCCCAGCCGGCCTTCTCTGGGGCCTCCCCGTCGTCAAGCCT
CTATCCTGTCTGTCCCCACCCCAGCTGTCCCCTGCCCAGGGAGCTGGCATAAAAGCA
CGAGGCCCGGCTCCCTGGGGCAGCTGCTTGAGAACAGAGACTGCTACCCCATCCTG
CCCATGCAGGCAGGCTCTTGCCAGCCCCGTTCTGACCCGTGTCCCCCCAGGCTCTGC
CTGGGCAGAAGACTCACCTTGGAGGAGTGGGCCCTGGAGTCCTGTCCCTCCCAGAA
GCCCCCAGGGTGGGATTTCTCAGGCTGCCAGGGCAGGCCCAGGCCTCAGGAAGAAG
GGGAGGCCCCTGGCCTCTCCGGGATCAGTCCTAGGACACAGGCTCAGCCTCAGGTT
GATGGGGGATGATGTGCTCCCGGGGCCTGCCTCCTGCACGGGGCTCCACGGAGCCC
AGCTCCCAGACACGCTACTAAGTGCCTAGGGTTGCCCGCTGTGGCCTGCTTCCAGGG
AGCAACAGAGAGGCCACCAAGCAGAGGCCCGTGGGGCTGAGGATGGAGCCGCCCC
CAGCCGACTCCAAGCCCGCAGAGGGCAGACGCCACCCTGGACTGCTCTCCCTGCCC
AGCTGGGCCTCTCTGGCCTATTCCTACCTTCCAGGCCCACTGCACTCCTGTCTGGGA
GGCCCTTATGAGGGCAGCCCAGCCCCCGCACCCACCCCCAACCAGAGAAGCACAGA
TCTTGGGGAGCTGCCCCACAAGCCCCGCTGGCCACCGAGGGCTGCAGCCGCTGCGC
TGCCGGCTTCTCCCCACCACCCTGCCACCTCCACTGTGATGTATGTCCGCTCCCTCGT
CTGTTCCCCCAGGATCTCGAAGTGACTCCGGGCTGAGCAGTGGGGCGGCTGGGGGA
GGGGTGACGATTCTCCTCAGGCTTTGGCCCTGCAAGCAAACCCACATATCTGCTCTG
TATGTAATAAATGTCTTAACGTCGTA.

By “MITOGEN-ACTIVATED PROTEIN KINASE 8-INTERACTING PROTEIN 3 (MAPK8IP3) polypeptide” is meant a polypeptide or fragment thereof having at least about 85% amino acid identity to UniProt Accession No. Q9UPT6, and which binds an antibody that specifically binds a MAPK8IP3 polypeptide. An exemplary MAPK8IP3 polypeptide sequence is provided below:

(SEQ ID NO: 2)
MMEIQMDEGGGVVVYQDDYCSGSVMSERVSGLAGSIYREFERLIHCYDEEVVKELMPL
VVNVLENLDSVLSENQEHEVELELLREDNEQLLTQYEREKALRRQAEEKFIEFEDALEQ
EKKELQIQVEHYEFQTRQLELKAKNYADQISRLEERESEMKKEYNALHQRHTEMIQTYV
EHIERSKMQQVGGNSQTESSLPGRRKERPTSLNVFPLADGTVRAQIGGKLVPAGDHWHL
SDLGQLQSSSSYQCPQDEMSESGQSSAAATPSTTGTKSNTPTSSVPSAAVTPLNESLQPL
GDYGVGSKNSKRAREKRDSRNMEVQVTQEMRNVSIGMGSSDEWSDVQDIIDSTPELDM
CPETRLDRTGSSPTQGIVNKAFGINTDSLYHELSTAGSEVIGDVDEGADLLGEFSVRDDF
FGMGKEVGNLLLENSQLLETKNALNVVKNDLIAKVDQLSGEQEVLRGELEAAKQAKV
KLENRIKELEEELKRVKSEAIIARREPKEEAEDVSSYLCTESDKIPMAQRRRFTRVEMAR
VLMERNQYKERLMELQEAVRWTEMIRASREHPSVQEKKKSTIWQFFSRLFSSSSSPPPA
KRPYPSVNIHYKSPTTAGFSQRRNHAMCPISAGSRPLEFFPDDDCTSSARREQKREQYRQ
VREHVRNDDGRLQACGWSLPAKYKQLSPNGGQEDTRMKNVPVPVYCRPLVEKDPTM
KLWCAAGVNLSGWRPNEDDAGNGVKPAPGRDPLTCDREGDGEPKSAHTSPEKKKAKE
LPEMDATSSRVWILTSTLTTSKVVIIDANQPGTVVDQFTVCNAHVLCISSIPAASDSDYPP
GEMFLDSDVNPEDPGADGVLAGITLVGCATRCNVPRSNCSSRGDTPVLDKGQGEVATIA
NGKVNPSQSTEEATEATEVPDPGPSEPETATLRPGPLTEHVFTDPAPTPSSGPQPGSENGP
EPDSSSTRPEPEPSGDPTGAGSSAAPTMWLGAQNGWLYVHSAVANWKKCLHSIKLKDS
VLSLVHVKGRVLVALADGTLAIFHRGEDGQWDLSNYHLMDLGHPHHSIRCMAVVYDR
VWCGYKNKVHVIQPKTMQIEKSFDAHPRRESQVRQLAWIGDGVWVSIRLDSTLRLYHA
HTHQHLQDVDIEPYVSKMLGTGKLGFSFVRITALLVAGSRLWVGTGNGVVISIPLTETV
VLHRGQLLGLRANKTSPTSGEGARPGGIIHVYGDDSSDRAASSFIPYCSMAQAQLCFHG
HRDAVKFFVSVPGNVLATLNGSVLDSPAEGPGPAAPASEVEGQKLRNVLVLSGGEGYID
FRIGDGEDDETEEGAGDMSQVKPVLSKAERSHIIVWQVSYTPE.

By “MITOGEN-ACTIVATED PROTEIN KINASE KINASE 4 (MAP2K4) polynucleotide” is meant a polynucleotide that encodes a MAP2K4 polypeptide or a fragment thereof having at least about 85% amino acid identity to NCBI Reference Sequence No: XM_011523976.2. An exemplary MAP2K4 polynucleotide sequence is provided below:

(SEQ ID NO: 3)
GAGCTTGCTGCATTGCAGCCGCCGCGGCGCCGCTCGGCTCTTCACTCCC
AACAATGGCGGCTCCGAGCCCGAGCGGCGGCGGCGGCTCCGGGGGCGGC
AGCGGCAGCGGCACCCCCGGCCCCGTAGGGTCCCCGGCGCCAGGCCACC
CGGCCGTCAGCAGCATGCAGGGCTTTCAGATAAACTTCTGTGAAAAGGC
ACAAAGTAAACGCAAAGCACTGAAGTTGAATTTTGCAAATCCACCTTTC
AAATCTACAGCAAGGTTTACTCTGAATCCCAATCCTACAGGAGTTCAAA
ACCCACACATAGAGAGACTGAGAACACACAGCATTGAGTCATCAGGAAA
ACTGAAGATCTCCCCTGAACAACACTGGGATTTCACTGCAGAGGACTTG
AAAGACCTTGGAGAAATTGGACGAGGAGCTTATGGTTCTGTCAACAAAA
TGGTCCACAAACCAAGTGGGCAAATAATGGCAGTTAAAAGAATTCGGTC
AACAGTGGATGAAAAAGAACAAAAACAACTTCTTATGGATTTGGATGTA
GTAATGCGGAGTAGTGATTGCCCATACATTGTTCAGTTTTATGGTGCAC
TCTTCAGAGAGGGTGACTGTTGGATCTGTATGGAACTCATGTCTACCTC
GTTTGATAAGTTTTACAAATATGTATATAGTGTATTAGATGATGTTATT
CCAGAAGAAATTTTAGGCAAAATCACTTTAGCAACTGTGAAAGCACTAA
ACCACTTAAAAGAAAACTTGAAAATTATTCACAGAGATATCAAACCTTC
CAATATTCTTCTGGACAGAAGTGGAAATATTAAGCTCTGTGACTTCGGC
ATCAGTGGACAGCTTGTGGACTCTATTGCCAAGACAAGAGATGCTGGCT
GTAGGCCATACATGGCACCTGAAAGAATAGACCCAAGCGCATCACGACA
AGGATATGATGTCCGCTCTGATGTCTGGAGTTTGGGGATCACATTGATA
ATAGGAGTCAACTATTATCTGCAACTGCACTAAGAATGGGAACAGGACA
AGCCAGCTTACCTGCAGTCAATTCATTGATGAAGGCACATAGGCTTCTC
CCATCTCAGGCAAGGTGTAGTATGTCCAGTAATACTGTATATAGTGTGT
ATATTAAGTATACGTATTTTTATATGTGGAAAATAGTTTCTACTTGCAA
TATTCTATTCACTGAATAGTAAAATCACAAGTGAATGAGCTGAAAGTAA
TTCAGTTTTTGAGGGACAATATATTAGTCCATTCTCACACTACTGTAAA
GAACTACATGAGACTGGGTAATTTATGAAGAGAGGTTTA.

By “MITOGEN-ACTIVATED PROTEIN KINASE KINASE 4 (MAP2K4) polypeptide” is meant a polypeptide or fragment thereof having at least about 85% amino acid identity to UniProt Accession No. P45985, and having kinase activity. An exemplary MAP2K4 polypeptide sequence is provided below:

(SEQ ID NO: 4)
MAAPSPSGGGGSGGGSGSGTPGPVGSPAPGHPAVSSMQGKRKALKLNFA
NPPFKSTARFTLNPNPTGVQNPHIERLRTHSIESSGKLKISPEQHWDFT
AEDLKDLGEIGRGAYGSVNKMVHKPSGQIMAVKRIRSTVDEKEQKQLLM
DLDVVMRSSDCPYIVQFYGALFREGDCWICMELMSTSFDKFYKYVYSVL
DDVIPEEILGKITLATVKALNHLKENLKIIHRDIKPSNILLDRSGNIKL
CDFGISGQLVDSIAKTRDAGCRPYMAPERIDPSASRQGYDVRSDVWSLG
ITLYELATGRFPYPKWNSVFDQLTQVVKGDPPQLSNSEEREFSPSFINF
VNLCLTKDESKRPKYKELLKHPFILMYEERAVEVACYVCKILDQMPATP
SSPMYVD

By “MITOGEN-ACTIVATED PROTEIN KINASE KINASE 7 (MAP2K7; Mkk7) polynucleotide” is meant a polynucleotide that encodes a MAP2K7 polypeptide or a fragment. An exemplary MAP2K7 sequence is provided at NCBI Reference Sequence No: NM_145185.4. An exemplary Mkk7 polynucleotide sequence is provided below:

(SEQ ID NO: 5)
AGTGCGGTGTTTGTCTGCCGGACTGACGGGCGGCCGGGCGGTGCGCGGCGGCGGTG
GCGGCGGGGAAGATGGCGGCGTCCTCCCTGGAACAGAAGCTGTCCCGCCTGGAAGC
AAAGCTGAAGCAGGAGAACCGGGAGGCCCGGCGGAGGATCGACCTCAACCTGGAT
ATCAGCCCCCAGCGGCCCAGGCCCACCCTGCAGCTCCCGCTGGCCAACGATGGGGG
CAGCCGCTCGCCATCCTCAGAGAGCTCCCCGCAGCACCCCACGCCCCCCGCCCGGCC
CCGCCACATGCTGGGGCTCCCGTCAACCCTGTTCACACCCCGCAGCATGGAGAGCAT
TGAGATTGACCAGAAGCTGCAGGAGATCATGAAGCAGACGGGCTACCTGACCATCG
GGGGCCAGCGCTACCAGGCAGAAATCAACGACCTGGAGAACTTGGGCGAGATGGG
CAGCGGCACCTGCGGCCAGGTGTGGAAGATGCGCTTCCGGAAGACCGGCCACGTCA
TTGCCGTTAAGCAAATGCGGCGCTCCGGGAACAAGGAGGAGAACAAGCGCATCCTC
ATGGACCTGGATGTGGTGCTGAAGAGCCACGACTGCCCCTACATCGTGCAGTGCTTT
GGGACGTTCATCACCAACACGGACGTCTTCATCGCCATGGAGCTCATGGGCACCTGC
GCTGAGAAGCTCAAGAAGCGGATGCAGGGCCCCATCCCCGAGCGCATTCTGGGCAA
GATGACAGTGGCGATTGTGAAGGCGCTGTACTACCTGAAGGAGAAGCACGGTGTCA
TCCACCGCGACGTCAAGCCCTCCAACATCCTGCTGGACGAGCGGGGCCAGATCAAG
CTCTGCGACTTCGGCATCAGCGGCCGCCTGGTGGACTCCAAAGCCAAGACGCGGAG
CGCCGGCTGTGCCGCCTACATGGCACCCGAGCGCATTGACCCCCCAGACCCCACCA
AGCCGGACTATGACATCCGGGCCGACGTATGGAGCCTGGGCATCTCGTTGGTGGAG
CTGGCAACAGGACAGTTTCCCTACAAGAACTGCAAGACGGACTTTGAGGTCCTCAC
CAAAGTCCTACAGGAAGAGCCCCCGCTTCTGCCCGGACACATGGGCTTCTCGGGGG
ACTTCCAGTCCTTCGTCAAAGACTGCCTTACTAAAGATCACAGGAAGAGACCAAAG
TATAATAAGCTACTTGAACACAGCTTCATCAAGCGCTACGAGACGCTGGAGGTGGA
CGTGGCGTCCTGGTTCAAGGATGTCATGGCGAAGACTGAGTCACCGCGGACTAGCG
GCGTCCTGAGCCAGCCCCACCTGCCCTTCTTCAGGTAGCTGCTTGGCGGCGGCCAGC
CCCACAGGGGGCCAGGGGCATGGCCACAGGCCCCCCTCCCCACTTGGCCACCCAGC
TGCCTGCCAGGGGAGACCTGGGACCTGGACGGCCACCTAGGACTGAGGACAGAGAG
TGGGGGGTGCCCACCCACCCCCCCCGCCCCGGGCCTACCAAGCCCCCGCCCTTCCCA
CCCCGGGGTCAGCCGGCCGTGTGCGTCCCCCGACAGACACTGTGAACGGAAGACAG
CAGGCCGCGATCAGAGTCGCTGTTCATTCAGCCGCAGCCTCTGGGCCGGGGCGGCC
CCCAGGGGCCAGGAGAGAGCCCTGGAGTCCCGCAGCCACCATGCACGCTCCCAGCG
TGCTGTGTCCTTCGCCACTCCCACGCGCCCGTTCCTCTTCCGTCGCCCTCTGTCCCCT
GCTCTACCTCTCTGTCCTTGTCTGGCTCTCCCGTCACCCTCCCTGCCTCTGTCTCTCTT
CTGGCCTGAGCCTGGGCCCAGCCACCTCCTGACGGGTCCCCTGGGTCTGCATAGGTC
TCCCATGGCGCAATGAGTCAGTGGCCCCCAGCCAGGCAGTGTGGGCATTGCCACTG
CGGCTGGACGGGGCTGCGCGCTCGCGCTCTCTCTCTCTCTCTCTCTCTCTCTTTGATC
TCAGGGGGTCCTTTTTGGAGTTTATTGTATTTTATTGTACTTGGTGGGGTGTTTGGGG
TGGGGGCGGAGGAGAGCTTGTTCTCGTGGGGTTGTCGGTACCTTCAGAAACTTTTAC
CAAAGTCACGATTAGCTGCTTGTGGTGGGGCCCCAACCGCCCTCGGGCACTGGGGA
GCTGGGCTGGGGCTGCTGCTCTGGGGTCTCCGGGGGCCACAGCTTGGGGTGAGTTG
AAGACCTCAGGGGATGTGGAGGGGTCTGCGGGGCCCTGGCCGCACAGGATGGCCTT
CAGGGAAGGTGGTCTTGGGGCATGGTGCAGAGCAGGTGACCGGAGGGAATCGGTG
ACGGAGCGGGGCCAAGGGAGGGGTCCGGAGGGAGTCAGGGATGGAGGGCAGAGGG
AGTGGATGTGGGGGTTTGAGGACGTGTGACAAGCTCCAGCAGGGGTGGGGGCCGGG
CTGAGGGTGGGGGTGCGAGGTGGTCACTCCCATCGTGCCCCTGGCCGTCCCTCCACT
CACCCACACCTGGCCCAGTCCACGTTGAGGTCCAGGACTGGGAAGGACCGGGTGAG
TGCACCGGGGACCCAGGCCAGGTGCCCCCCGGAGCCTGCTGGGGTGGCCAGAGCAG
GAGGGGGTGTGTTTCCTTTTTGTGGGTGTTGCATGCAAATCAAGTGGACAAGAAAAA
ATAACAAAACAAAAAACAAGAAAAAAAAAACACAAAACCCCGTAAAATCACAAAG
AAAATCCAACACCAAAGGCGCAGAAGCCGGCTGGCCGTGGTGGGGGCAGCGTAGG
CGTAGCATCCCTCTCCTCTCACTTAGCCTGTTGACTCTTGTTATTATCATGATATTCA
CAAAACGCCGCATGTTTAAAAAGTCATAGATGTCATCTTCTCTCTGCCCCCAGGGAG
GAAAGCCACCTTCTCTTGCCCCTTGGCCCCTTTGTCAGGGGCCAGGGGTCTGCCGGG
TGGGGGTGCCAACAGGCCTGGCCCTTTCCTCCCCTGCATCCAGCCATGGGGGCCTCT
GCGATTGCCGGAAGGTTGCATGGCTGGTCCCAGGGCCAGCACAGGCCCGAGGCCGG
GCTGCCTGGTTTTATTTTTATTTAACTTTATTTTCTGTTTTATGAGTGTGTGTCCGCCC
ACCCCCACCCCCTTCAGTGTTAAGTGGGGAGCCCTGGGGGAGTCTCTCCTGCCTCCC
AGCCTCTCCCAAGACCTCCCCCCTCGTCACCAGCCATCCCTCTGGACCAGGCAGAGG
GCGGACCGGGTGGGCAGGGGCCTGAGGGTGGCTCGGGCCAGCCCACCAGCCAATGG
ACCCCTCCTCAGGCCGCCAGTGTCGCCCTGCCCCTTTTTAAAACAAAATGCCCTCGT
TTGTAAACCCTTAGACGCTTGAGAATAAACCCCTTCCTTTTCTTCCA.

By “MITOGEN-ACTIVATED PROTEIN KINASE KINASE 7 (MAP2K7; Mkk7) polypeptide” is meant a polypeptide or fragment thereof having at least about 85% amino acid identity to UniProt Accession No. O14733, and having kinase activity. An exemplary Mkk7 polypeptide sequence is provided below:

(SEQ ID NO: 6)
MAASSLEQKLSRLEAKLKQENREARRRIDLNLDISPQRPRPTLQLPLAN
DGGSRSPSSESSPQHPTPPARPRHMLGLPSTLFTPRSMESIEIDQKLQE
IMKQTGYLTIGGQRYQAEINDLENLGEMGSGTCGQVWKMRFRKTGHVIA
VKQMRRSGNKEENKRILMDLDVVLKSHDCPYIVQCFGTFITNTDVFIAM
ELMGTCAEKLKKRMQGPIPERILGKMTVAIVKALYYLKEKHGVIHRDVK
PSNILLDERGQIKLCDFGISGRLVDSKAKTRSAGCAAYMAPERIDPPDP
TKPDYDIRADVWSLGISLVELATGQFPYKNCKTDFEVLTKVLQEEPPLL
PGHMGFSGDFQSFVKDCLTKDHRKRPKYNKLLEHSFIKRYETLEVDVAS
WFKDVMAKTESPRTSGVLSQPHLPFFR.

By “MITOGEN-ACTIVATED PROTEIN KINASE KINASE KINASE 11 (Map3k11) gene” is meant a polynucleotide that encodes a Map3k11 polypeptide or a fragment thereof. An exemplary Map3k11 sequence is provided at NCBI Reference Sequence No: XR_428915.2.

By “MITOGEN-ACTIVATED PROTEIN KINASE KINASE KINASE 11 (Map3k11) polypeptide” is meant a polypeptide or fragment thereof having at least about 85% amino acid identity to UniProt Accession No. Q16584, and having kinase activity.

By “Mitogen-Activated Protein Kinase Kinase Kinase 19 (Map3k19) polynucleotide” is meant a polynucleotide that encodes a Map3k19 polypeptide or a fragment thereof. An exemplary Map3k19 sequence is provided at NCBI Reference Sequence No: NM_001018045.

By “Mitogen-Activated Protein Kinase Kinase Kinase 19 (Map3k19) polypeptide” is meant a polypeptide or fragment thereof having at least about 85% amino acid identity to UniProt Accession No. Q56UN5, and having kinase activity.

By “p21 PROTEIN-ACTIVATED KINASE 4 (Pak4) polynucleotide” is meant a polynucleotide that encodes a Pak4 polypeptide or a fragment thereof. An exemplary Pak4 sequence is provided at NCBI Reference Sequence No: NM_001014832.2.

By “p21 PROTEIN-ACTIVATED KINASE 4 (Pak4) polypeptide” is meant a polypeptide or fragment thereof having at least about 85% amino acid identity to UniProt Accession No. O96013, and having kinase activity.

By “PAIRED BOX GENE 6 (Pax6) polynucleotide” is meant a polynucleotide that encodes a Pax6 polypeptide or a fragment thereof having at least about 85% amino acid identity to NCBI Reference Sequence No: NM_001310160.2.

By “PAIRED BOX GENE 6 (Pax6) polypeptide” is meant a polypeptide or fragment thereof having at least about 85% amino acid identity to UniProt Accession No. P26367, and which binds DNA and/or regulates transcription.

By “PAS DOMAIN-CONTAINING SERINE/THREONINE KINASE (Pask) polynucleotide” is meant a polynucleotide that encodes a Pask polypeptide or a fragment thereof. An exemplary Pask sequence is provided at NCBI Reference Sequence No: XM_005246991.1.

By “PAS DOMAIN-CONTAINING SERINE/THREONINE KINASE (Pask) polypeptide” is meant a polypeptide or fragment thereof having at least about 85% amino acid identity to UniProt Accession No. Q96RG2, and having kinase activity.

By “Pdp1 polynucleotide” is meant a polynucleotide that encodes a Pdp1 polypeptide or a fragment thereof. An exemplary Pdp1 polynucleotide sequence is provided at NCBI Reference Sequence No: NM_001161779.2.

By “PHD FINGER PROTEIN 5A (Phf5a) polynucleotide” is meant a polynucleotide that encodes a Phf5a polypeptide or a fragment thereof having at least about 85% amino acid identity to NCBI Reference Sequence No: NM_032758.4.

By “PHD FINGER PROTEIN 5A (Phf5a) polypeptide” is meant a polypeptide or fragment thereof having at least about 85% amino acid identity to UniProt Accession No. Q7RTV0, and which binds an antibody that specifically binds a Phf5a polypeptide.

By “phosphatidylinositol 4-kinase (Pi4kb) polynucleotide” is meant a polynucleotide that encodes a Pi4kb polypeptide or a fragment thereof. An exemplary Pi4kb polynucleotide sequence is provided at NCBI Reference Sequence No: NM_001198774.1.

By “phosphatidylinositol 4-kinase (Pi4kb) polypeptide” is meant a polypeptide or fragment thereof having at least about 85% amino acid identity to UniProt Accession No. Q9UBF8, and having kinase activity.

By “PHOSPHOGLYCERATE KINASE 1 (Pgk1) polynucleotide” is meant a polynucleotide that encodes a Pgk1 polypeptide or a fragment thereof. An exemplary Pgk1 sequence is provided at NCBI Reference Sequence No: NM_000291.

By “PHOSPHOGLYCERATE KINASE 1 (Pgk1) polypeptide” is meant a polypeptide or fragment thereof having at least about 85% amino acid identity to UniProt Accession No. P00558, and having kinase activity.

By “PHOSPHOLIPASE D2 (Pld2) polynucleotide” is meant a polynucleotide that encodes a Pld2 polypeptide or a fragment thereof. An exemplary Pld2 polynucleotide sequence is provided at NCBI Reference Sequence No: XR_001752537.2.

By “PHOSPHOLIPASE D2 (Pld2) polypeptide” is meant a polypeptide or fragment thereof having at least about 85% amino acid identity to UniProt Accession No. O14939, and having lipase activity.

By “PRKC, APOPTOSIS, WT1, REGULATOR (Pawr) polynucleotide” is meant a polynucleotide that encodes a Pawr polypeptide or a fragment thereof. An exemplary Pawr polynucleotide sequence is provided at NCBI Reference Sequence No: XM_017019379.1.

By “PRKC, APOPTOSIS, WTT, REGULATOR (Pawr) polypeptide” is meant a polypeptide or fragment thereof having at least about 85% amino acid identity to UniProt Accession No. Q961Z0, and which regulates transcription.

By “PROTEIN KINASE, AMP-ACTIVATED, NONCATALYTIC, GAMMA-2 (Prkag2) polynucleotide” is meant a polynucleotide that encodes a Prkag2 polypeptide or a fragment thereof. An exemplary Prkag2 polynucleotide sequence is provided at NCBI Reference Sequence No: XM_017012280.2.

By “PROTEIN KINASE, AMP-ACTIVATED, NONCATALYTIC, GAMMA-2 (Prkag2) polypeptide” is meant a polypeptide or fragment thereof having at least about 85% amino acid identity to UniProt Accession No. Q9UGJ0, and having kinase activity.

By “PROTEIN KINASE, SERINE/ARGININE-SPECIFIC, 2 (Srpk2) polynucleotide” is meant a polynucleotide that encodes a Srpk2 polypeptide or a fragment thereof. An exemplary Srpk2 polynucleotide sequence is provided at NCBI Reference Sequence No: XM_024446894.1.

By “PROTEIN KINASE, SERINE/ARGININE-SPECIFIC, 2 (Srpk2) polypeptide” is meant a polypeptide or fragment thereof having at least about 85% amino acid identity to UniProt Accession No. P78362, and having kinase activity.

By “PROTEIN-TYROSINE PHOSPHATASE, NONRECEPTOR-TYPE, 2 (Ptpn2) polynucleotide” is meant a polynucleotide that encodes a Ptpn2 polypeptide or a fragment thereof. An exemplary Ptpn2 polynucleotide sequence is provided at NCBI Reference Sequence No: NM_080423.3.

By “PROTEIN-TYROSINE PHOSPHATASE, NONRECEPTOR-TYPE, 2 (Ptpn2) polypeptide” is meant a polypeptide or fragment thereof having at least about 85% amino acid identity to UniProt Accession No. P17706, and having phosphatase activity.

By “PYRUVATE DEHYDROGENASE PHOSPHATASE CATALYTIC SUBUNIT 1Pdp1 polypeptide” is meant a polypeptide or fragment thereof having at least about 85% amino acid identity to UniProt Accession No. Q9P0J1, and having phosphatase activity.

By “PYRUVATE DEHYDROGENASE PHOSPHATASE CATALYTIC SUBUNIT 2 (Pdp2) polynucleotide” is meant a polynucleotide that encodes a Pdp2 polypeptide or a fragment thereof. An exemplary Pdp2 polynucleotide sequence is provided at NCBI Reference Sequence No: NM_001329928.2.

By “PYRUVATE DEHYDROGENASE PHOSPHATASE CATALYTIC SUBUNIT 2 (Pdp2) polypeptide” is meant a polypeptide or fragment thereof having at least about 85% amino acid identity to UniProt Accession No. Q9P2J9, and having phosphatase activity.

By “PYRUVATE KINASE, LIVER AND RED BLOOD CELL (Pklr) polynucleotide” is meant a polynucleotide that encodes a Pklr polypeptide or a fragment thereof. An exemplary Pklr polynucleotide sequence is provided at NCBI Reference Sequence No: XM_011509640.3.

By “PYRUVATE KINASE, LIVER AND RED BLOOD CELL (Pklr) polypeptide” is meant a polypeptide or fragment thereof having at least about 85% amino acid identity to UniProt Accession No. P30613, and having kinase activity.

By “RECEPTOR-INTERACTING SERINE/THREONINE KINASE 1 (Ripk1) polynucleotide” is meant a polynucleotide that encodes a Ripk1 polypeptide or a fragment thereof. An exemplary Ripk1 polynucleotide sequence is provided at NCBI Reference Sequence No: XM_017011403.1.

By “RECEPTOR-INTERACTING SERINE/THREONINE KINASE 1 (Ripk1) polypeptide” is meant a polypeptide or fragment thereof having at least about 85% amino acid identity to UniProt Accession No. Q13546, and having kinase activity.

By “RECEPTOR-INTERACTING SERINE/THREONINE KINASE 3 (Ripk3) polynucleotide” is meant a polynucleotide that encodes a Ripk3 polypeptide or a fragment thereof. An exemplary Ripk3 polynucleotide sequence is provided at NCBI Reference Sequence No: NM 006871.

By “RECEPTOR-INTERACTING SERINE/THREONINE KINASE 3 (Ripk3) polypeptide” is meant a polypeptide or fragment thereof having at least about 85% amino acid identity to UniProt Accession No. Q9Y572, and having kinase activity.

By “RETINOBLASTOMA-BINDING PROTEIN 7 (Rbbp7) polynucleotide” is meant a polynucleotide that encodes a Rbbp7 polypeptide or a fragment thereof having at least about 85% amino acid identity to NCBI Reference Sequence No: NM_002893.4.

By “RETINOBLASTOMA-BINDING PROTEIN 7 (Rbbp7) polypeptide” is meant a polypeptide or fragment thereof having at least about 85% amino acid identity to UniProt Accession No. Q16576, and which binds an antibody that specifically binds a Rbbp7 polypeptide.

By “RING FINGER PROTEIN 141 (Rnf141) polynucleotide” is meant a polynucleotide that encodes a Rnf141 polypeptide or a fragment thereof having at least about 85% amino acid identity to NCBI Reference Sequence No: NM_016422.4.

By “RING FINGER PROTEIN 141 (Rnf141) polypeptide” is meant a polypeptide or fragment thereof having at least about 85% amino acid identity to UniProt Accession No. Q8WVD5, and which binds an antibody that specifically binds a Rnf141 polypeptide.

By “RNA GUANYLYLTRANSFERASE AND 5-PRIME-PHOSPHATASE (Rngtt) polynucleotide” is meant a polynucleotide that encodes a Rngtt polypeptide or a fragment thereof. An exemplary Rngtt polynucleotide sequence is provided at NCBI Reference Sequence No: XM_017011401.2.

By “RNA GUANYLYLTRANSFERASE AND 5-PRIME-PHOSPHATASE (Rngtt) polypeptide” is meant a polypeptide or fragment thereof having at least about 85% amino acid identity to UniProt Accession No. O60942, and having phosphatase activity.

By “SERINE/THREONINE PROTEIN KINASE 10 (Stk10) polynucleotide” is meant a polynucleotide that encodes a Stk10polypeptide or a fragment thereof. An exemplary Stk10polynucleotide sequence is provided at NCBI Reference Sequence No: NM_005990.4.

By “SERINE/THREONINE PROTEIN KINASE 10 (Stk10) polypeptide” is meant a polypeptide or fragment thereof having at least about 85% amino acid identity to UniProt Accession No. O94804, and having kinase activity.

By “SERINE/THREONINE PROTEIN KINASE 11 (Lkb1) polynucleotide” is meant a polynucleotide that encodes a Lkb1 polypeptide or a fragment thereof. An exemplary Lkb1polynucleotide sequence is provided at NCBI Reference Sequence No: NM_000455. An exemplary Lkb1 polynucleotide sequence is provided below:

(SEQ ID NO: 7)
GAGGTAAACAAGATGGCGGCGGCGTGTCGGGCGCGGAAGGGGGAGGCGGCCCGGG
GCGCCCGCGAGTGAGGCGCGGGGCGGCGAAGGGAGCGCGGGTGGCGGCACTTGCT
GCCGCGGCCTTGGATGGGCTGGGCCCCCCTCGCCGCTCCGCCTCCTCCACACGCGCG
GCGGCCGCGGCGAGGGGGACGCGCCGCCCGGGGCCCGGCACCTTCGGGAACCCCCC
GGCCCGGAGCCTGCGGCCTGCGCCGCCTCGGCCGCCGGGAGCCCCGTGGAGCCCCC
GCCGCCGCGCCGCCCCGCGGACCGGACGCTGAGGGCACTCGGGGCGGGGCGCGCGC
TCGGGCAGACGTTTGCGGGGAGGGGGGCGCCTGCCGGGCCCCGGCGACCACCTTGG
GGGTCGCGGGCCGGCTCGGGGGGCGCCCAGTGCGGGCCCTCGCGGGCGCCGGGCAG
CGACCAGCCCTGAGCGGAGCTGTTGGCCGCGGCGGGAGGCCTCCCGGACGCCCCCA
GCCCCCCGAACGCTCGCCCGGGCCGGCGGGAGTCGGCGCCCCCCGGGAGGTCCGCT
CGGTCGTCCGCGGCGGAGCGTTTGCTCCTGGGACAGGCGGTGGGACCGGGGCGTCG
CCGGAGACGCCCCCAGCGAAGTTGGGCTCTCCAGGTGTGGGGGTCCCGGGGGGTAG
CGACGTCGCGGACCCGGCCTGTGGGATGGGCGGCCCGGAGAAGACTGCGCTCGGCC
GTGTTCATACTTGTCCGTGGGCCTGAGGTCCCCGGAGGATGACCTAGCACTGAAAAG
CCCCGGCCGGCCTCCCCAGGGTCCCCGAGGACGAAGTTGACCCTGACCGGGCCGTC
TCCCAGTTCTGAGGCCCGGGTCCCACTGGAACTCGCGTCTGAGCCGCCGTCCCGGAC
CCCCGGTGCCCGCCGGTCCGCAGACCCTGCACCGGGCTTGGACTCGCAGCCGGGAC
TGACGTGTAGAACAATCGTTTCTGTTGGAAGAAGGGTTTTTCCCTTCCTTTTGGGGTT
TTTGTTGCCTTTTTTTTTTCTTTTTTCTTTGTAAAATTTTGGAGAAGGGAAGTCGGAAC
ACAAGGAAGGACCGCTCACCCGCGGACTCAGGGCTGGCGGCGGGACTCCAGGACCC
TGGGTCCAGCATGGAGGTGGTGGACCCGCAGCAGCTGGGCATGTTCACGGAGGGCG
AGCTGATGTCGGTGGGTATGGACACGTTCATCCACCGCATCGACTCCACCGAGGTCA
TCTACCAGCCGCGCCGCAAGCGGGCCAAGCTCATCGGCAAGTACCTGATGGGGGAC
CTGCTGGGGGAAGGCTCTTACGGCAAGGTGAAGGAGGTGCTGGACTCGGAGACGCT
GTGCAGGAGGGCCGTCAAGATCCTCAAGAAGAAGAAGTTGCGAAGGATCCCCAACG
GGGAGGCCAACGTGAAGAAGGAAATTCAACTACTGAGGAGGTTACGGCACAAAAA
TGTCATCCAGCTGGTGGATGTGTTATACAACGAAGAGAAGCAGAAAATGTATATGG
TGATGGAGTACTGCGTGTGTGGCATGCAGGAAATGCTGGACAGCGTGCCGGAGAAG
CGTTTCCCAGTGTGCCAGGCCCACGGGTACTTCTGTCAGCTGATTGACGGCCTGGAG
TACCTGCATAGCCAGGGCATTGTGCACAAGGACATCAAGCCGGGGAACCTGCTGCT
CACCACCGGTGGCACCCTCAAAATCTCCGACCTGGGCGTGGCCGAGGCACTGCACC
CGTTCGCGGCGGACGACACCTGCCGGACCAGCCAGGGCTCCCCGGCTTTCCAGCCG
CCCGAGATTGCCAACGGCCTGGACACCTTCTCCGGCTTCAAGGTGGACATCTGGTCG
GCTGGGGTCACCCTCTACAACATCACCACGGGTCTGTACCCCTTCGAAGGGGACAAC
ATCTACAAGTTGTTTGAGAACATCGGGAAGGGGAGCTACGCCATCCCGGGCGACTG
TGGCCCCCCGCTCTCTGACCTGCTGAAAGGGATGCTTGAGTACGAACCGGCCAAGA
GGTTCTCCATCCGGCAGATCCGGCAGCACAGCTGGTTCCGGAAGAAACATCCTCCG
GCTGAAGCACCAGTGCCCATCCCACCGAGCCCAGACACCAAGGACCGGTGGCGCAG
CATGACTGTGGTGCCGTACTTGGAGGACCTGCACGGCGCGGACGAGGACGAGGACC
TCTTCGACATCGAGGATGACATCATCTACACTCAGGACTTCACGGTGCCCGGACAGG
TCCCAGAAGAGGAGGCCAGTCACAATGGACAGCGCCGGGGCCTCCCCAAGGCCGTG
TGTATGAACGGCACAGAGGCGGCGCAGCTGAGCACCAAATCCAGGGCGGAGGGCC
GGGCCCCCAACCCTGCCCGCAAGGCCTGCTCCGCCAGCAGCAAGATCCGCCGGCTG
TCGGCCTGCAAGCAGCAGTGAGGCTGGCCGCCTGCAGCCCGTGTCCAGGAGCCCCG
CCAGGTGCCCGCGCCAGGCCCTCAGTCTTCCTGCCGGTTCCGCCCGCCCTCCCGGAG
AGGTGGCCGCCATGCTTCTGTGCCGACCACGCCCCAGGACCTCCGGAGCGCCCTGCA
GGGCCGGGCAGGGGGACAGCAGGGACCGGGCGCAGCCCTCCCCCCTCGGCCGCCCG
GCAGTGCACGCGGCTTGTTGACTTCGCAGCCCCGGGCGGAGCCTTCCCGGGCGGGC
GTGGGAGGAGGGAGGCGGCCTCCATGCACTTTATGTGGAGACTACTGGCCCCGCCC
GTGGCCTCGTGCTCCGCAGGGCGCCCAGCGCCGTCCGGCGGCCCCGCCGCAGACCA
GCTGGCGGGTGTGGAGACCAGGCTCCTGACCCCGCCATGCATGCAGCGCCACCTGG
AAGCCGCGCGGCCGCTTTGGTTTTTTGTTTGGTTGGTTCCATTTTCTTTTTTTCTTTTT
TTTTTTAAGAAAAAATAAAAGGTGGATTTGAGCTGTGGCTGTGAGGGGTGTTTGGGA
GCTGCTGGGTGGCAGGGGGGCTGTGGGGTCGGGCTCACGTCGCGGCCGCCTTTGCG
CTCTCGGGTCACCCTGCTTTGGCGGCCCGGCCGGAGGGCAGGACCCTCACCTCTCCC
CCAAGGCCACTGCGCTCTTGGGACCCCAGAGAAAACCCGGAGCAAGCAGGAGTGTG
CGGTCAATATTTATATCATCCAGAAAAGAAAAACACGAGAAACGCCATCGCGGGAT
GGTGCAGACGCGGCGGGGACTCGGAGGGTGCCGTGCGGGCGAGGCCGCCCAAATTT
GGCAATAAATAAAGCTTGGGAAGCTTGGA.

By “SERINE/THREONINE PROTEIN KINASE 11 (Lkb1) polypeptide” is meant a 10 polypeptide or fragment thereof having at least about 85% amino acid identity to UniProt Accession No. Q15831, and having kinase activity. An exemplary Lkb1 polypeptide sequence is provided below:

(SEQ ID NO: 8)
MEVVDPQQLGMFTEGELMSVGMDTFIHRIDSTEVIYQPRRKRAKLIGKY
LMGDLLGEGSYGKVKEVLDSETLCRRAVKILKKKKLRRIPNGEANVKKE
IQLLRRLRHKNVIQLVDVLYNEEKQKMYMVMEYCVCGMQEMLDSVPEKR
FPVCQAHGYFCQLIDGLEYLHSQGIVHKDIKPGNLLLTTGGTLKISDLG
VAEALHPFAADDTCRTSQGSPAFQPPEIANGLDTFSGFKVDIWSAGVTL
YNITTGLYPFEGDNIYKLFENIGKGSYAIPGDCGPPLSDLLKGMLEYEP
AKRFSIRQIRQHSWFRKKHPPAEAPVPIPPSPDTKDRWRSMTVVPYLED
LHGADEDEDLFDIEDDIIYTQDFTVPGQVPEEEASHNGQRRGLPKAVCM
NGTEAAQLSTKSRAEGRAPNPARKACSASSKIRRLSACKQQ.

By “SERINE/THREONINE PROTEIN KINASE 38-LIKE PROTEIN (Stk38l) polynucleotide” is meant a polynucleotide that encodes a Stk381 polypeptide or a fragment thereof. An exemplary Stk10polynucleotide sequence is provided at NCBI Reference Sequence No: XM_005253342.4.

By “SERINE/THREONINE PROTEIN KINASE 38-LIKE PROTEIN (Stk38l) polypeptide” is meant a polypeptide or fragment thereof having at least about 85% amino acid identity to UniProt Accession No. Q9Y2H1, and having kinase activity.

By “SERTA DOMAIN-CONTAINING PROTEIN 1 (Sertad1) polynucleotide” is meant a polynucleotide that encodes a Sertad1 polypeptide or a fragment thereof having at least about 85% amino acid identity to NCBI Reference Sequence No: NM_013376.4.

By “SERTA DOMAIN-CONTAINING PROTEIN 1 (Sertad1) polypeptide” is meant a polypeptide or fragment thereof having at least about 85% amino acid identity to UniProt Accession No. Q9UHV2, and which binds an antibody that specifically binds a Sertad1 polypeptide.

By “SERUM RESPONSE FACTOR (Srf) polynucleotide” is meant a polynucleotide that encodes a Srf polypeptide or a fragment thereof having at least about 85% amino acid identity to NCBI Reference Sequence No: NM_003131.4.

By “SERUM RESPONSE FACTOR (Srf) polypeptide” is meant a polypeptide or fragment thereof having at least about 85% amino acid identity to UniProt Accession No. P11831, and having transcriptional regulatory activity.

By “SH3-DOMAIN KINASE-BINDING PROTEIN 1 (Sh3kbp1) polynucleotide” is meant a polynucleotide that encodes a Sh3kbp1 polypeptide or a fragment thereof. An exemplary Sh3kbp1 polynucleotide sequence is provided at NCBI Reference Sequence No: NM_001353891.2.

By “SH3-DOMAIN KINASE-BINDING PROTEIN 1 (Sh3kbp1) polypeptide” is meant a polypeptide or fragment thereof having at least about 85% amino acid identity to UniProt Accession No. Q96B97, and having kinase activity.

By “SIM bHLH TRANSCRIPTION FACTOR 1 (Sim1) polynucleotide” is meant a polynucleotide that encodes a Sim1 polypeptide or a fragment thereof having at least about 85% amino acid identity to NCBI Reference Sequence No: NM_005068.3.

By “SIM bHLH TRANSCRIPTION FACTOR 1 (Sim1) polypeptide” is meant a polypeptide or fragment thereof having at least about 85% amino acid identity to UniProt Accession No. P81133, and having transcriptional regulatory activity.

By “SIN3 TRANSCRIPTION REGULATOR FAMILY MEMBER A (Sin3a) polynucleotide” is meant a polynucleotide that encodes a Sin3a polypeptide or a fragment thereof having at least about 85% amino acid identity to NCBI Reference Sequence No: NM_015477.3.

By “SIN3 TRANSCRIPTION REGULATOR FAMILY MEMBER A (Sin3a) polypeptide” is meant a polypeptide or fragment thereof having at least about 85% amino acid identity to UniProt Accession No. Q96ST3, and having transcription regulatory activity.

By “SMG1 NONSENSE-MEDIATED mRNA DECAY-ASSOCIATED P13K-RELATED KINASE (Smg1) polynucleotide” is meant a polynucleotide that encodes a Smg1 polypeptide or a fragment thereof. An exemplary Smg1polynucleotide sequence is provided at NCBI Reference Sequence No: XM_017023066.2.

By “SMG1 NONSENSE-MEDIATED mRNA DECAY-ASSOCIATED P13K-RELATED KINASE (Smg1) polypeptide” is meant a polypeptide or fragment thereof having at least about 85% amino acid identity to UniProt Accession No. Q96Q15, and having kinase activity.

By “SNF-RELATED KINASE (Snrk) polynucleotide” is meant a polynucleotide that encodes a Snrk polypeptide or a fragment thereof. An exemplary Snrk polynucleotide sequence is provided at NM_001100594.2. An exemplary Snrk polynucleotide sequence is provided below:

(SEQ ID NO: 9)
AGCTACCCGGCACCCCCTCCCCGCGGCCGGCAGCCCGCTCGGTATTATGATTAGCGC
TGGGTGCGGGGTTTCGGCGGCCGGGAGGGAGTTGTCGGCGCCGCGGCCGCTGCGGA
CGGACGCTCGCCTGCCGGCTGAGATATCCATGACGACATTGAAAATGAATTTTTTGT
ATTCACCAGATATTCTTATATGAGAAGATCTATTTTAAACAGTCTAAATATTTTTTCT
TCTGTTGGACCAGCATGGCAGGATTTAAGCGAGGGTATGATGGAAAGATTGCTGGA
TTATATGATCTGGATAAAACCTTGGGTCGAGGCCATTTTGCCGTGGTTAAACTTGCC
AGGCATGTCTTTACGGGTGAAAAGGTGGCAGTAAAAGTTATTGACAAGACAAAACT
GGACACTCTAGCTACTGGTCATCTTTTCCAGGAAGTGAGATGCATGAAACTAGTGCA
GCATCCTAACATCGTCCGCCTTTATGAAGTTATTGACACCCAGACCAAACTATATCT
TATTCTAGAACTTGGGGATGGAGGAGATATGTTTGATTATATAATGAAACATGAGG
AGGGTCTTAATGAAGACTTGGCCAAGAAGTATTTTGCTCAGATAGTTCATGCTATAT
CTTATTGCCATAAACTCCATGTGGTTCACAGAGACTTAAAACCAGAGAATGTAGTCT
TCTTTGAAAAACAAGGTCTTGTAAAGTTGACAGACTTTGGGTTCAGCAACAAATTTC
AACCAGGGAAGAAGCTCACTACAAGCTGTGGATCTCTTGCATATTCCGCTCCAGAA
ATTCTGCTTGGTGATGAGTATGATGCACCTGCAGTAGATATTTGGAGTCTGGGAGTG
ATCCTTTTCATGTTGGTGTGTGGGCAGCCGCCCTTTCAAGAAGCCAATGACAGTGAA
ACACTGACAATGATCATGGATTGCAAATATACAGTACCATCCCATGTGTCTAAAGAG
TGTAAAGACCTAATCACACGGATGCTACAGAGAGATCCCAAGAGAAGGGCTTCTTT
AGAAGAGATTGAAAATCATCCTTGGCTTCAGGGAGTGGACCCTTCACCAGCTACAA
AGTATAACATTCCCCTTGTGTCATACAAAAATCTCTCGGAAGAGGAGCACAACAGC
ATCATTCAGCGCATGGTGCTTGGGGACATAGCGGATCGAGACGCCATTGTAGAAGC
CCTGGAAACCAACAGGTATAACCATATCACAGCCACATACTTCCTGCTGGCTGAAA
GGATCCTGAGAGAAAAGCAAGAGAAAGAAATACAGACCAGATCTGCAAGCCCGAG
CAATATCAAGGCCCAGTTTAGGCAGTCATGGCCAACCAAAATTGATGTACCCCAGG
ACCTTGAGGATGACCTCACGGCCACTCCTTTGTCCCACGCGACTGTCCCTCAGTCTC
CTGCTCGGGCTGCTGACAGTGTCCTCAATGGCCACAGGAGCAAAGGCCTGTGTGACT
CAGCTAAGAAAGATGACCTCCCTGAGTTGGCTGGACCAGCACTCTCTACGGTGCCAC
CCGCAAGCTTAAAACCCACAGCCAGTGGGCGGAAGTGTCTGTTCAGGGTGGAAGAA
GATGAAGAGGAAGATGAGGAGGACAAGAAACCCATGTCCCTCTCAACACAAGTGGT
TTTGCGCCGGAAGCCATCTGTAACCAACCGCCTGACATCCAGGAAGAGTGCGCCCG
TCCTCAACCAGATCTTTGAGGAAGGGGAATCTGACGATGAGTTTGACATGGATGAG
AATCTGCCTCCCAAGTTGAGCAGGTTAAAGATGAATATAGCTTCTCCAGGTACAGTT
CACAAACGCTACCACCGGAGGAAAAGTCAGGGCCGGGGCTCCAGCTGCAGTAGTTC
GGAGACCAGTGATGATGATTCTGAAAGCCGGCGGCGGCTCGATAAAGATAGCGGGT
TCACCTACTCCTGGCACCGACGGGATAGCAGCGAGGGGCCCCCTGGCAGTGAGGGG
GATGGCGGGGGCCAGAGCAAGCCAAGCAATGCCAGTGGAGGGGTGGACAAGGCCA
GCCCCAGTGAGAACAATGCTGGTGGGGGCAGTCCCTCCAGCGGCTCGGGTGGCAAC
CCCACCAATACATCGGGTACCACACGCCGCTGTGCCGGCCCCAGCAACTCCATGCA
GCTGGCCTCTCGCAGTGCTGGGGAGCTCGTTGAGAGCCTCAAACTCATGAGCCTCTG
CCTCGGCTCCCAGCTTCATGGGAGCACCAAGTACATTATTGATCCACAGAATGGCTT
GTCATTTTCCAGTGTGAAAGTCCAAGAGAAATCTACGTGGAAAATGTGCATTAGCTC
CACAGGGAATGCAGGGCAGGTCCCTGCAGTGGGCGGCATAAAGTTTTTCTCTGACC
ACATGGCAGATACCACCACTGAATTGGAACGGATAAAGAGCAAGAACCTGAAAAAT
AACGTGCTGCAGCTACCTCTGTGCGAAAAGACCATCTCTGTGAACATCCAGCGGAA
CCCTAAGGAGGGGCTGCTGTGCGCATCCAGCCCAGCCAGCTGTTGCCATGTCATCTG
ACTGTGGCCCCATCTGGCCGCTAGCACGCTTCCTGCTCAGAGCAGTGAAGACCGGCT
CACTTCACTGTTCCATTTGGTTTTACTATTTTAAAGTGGGCGTTAGGAGCAATTATTT
ATTACCTTTCCATTTGTTCGCCTGATGATGTGACAATGCATGGTCTTTGTGCATGCTG
CTAGACACTTTTCTTTCCCAGCCGAAAAGCCTATTATGTAATTTTTACATTCATAATT
TTAATGTGGATGATCAGGATTAAATCAAGATATATATCTGGAACCTCTTATAAATGG
AGCACTTAGAAATTTGTTGTTCTGCACTTAACCTAGAGAGAGAAAAAATGCTTTTCT
TTGTGAAAAATCTGAATTCCTGTCCTGACCTTCTGTGATGTGGAAACCCTAGGCTCT
GAGACACACTCTCTGGTGTCTGAGACAGAACCAAAGCAATAACGTTGTGATGCCCA
CAGGCCTGGAGCCAGCTAGCGACCTTGTGCCGCCCAGCTGTCCATGGCCCGTGCAG
AGCAGAGGACAGTGAGTGTCTGCACTGAGAACCTTAAACCACAGTTGAACATACCC
ACACCTGTTTGTCTTAAGCTATAGTGTAAAAACAAAGTTTGGGCTCTGAAAATTTAA
CTGAAAAAGATTTCCTTGTTTTTGTAATAGGTGAGATAAAGTACTTAGATTTATAAG
GCAGCTTCCCCTGTAGTGATAAATTACAAGCAGACAATCTTATTTTGTAATGTGATG
AAGTGATGATGTCTTAACTCTACTTAGAGAGTGTATGTCTGTCTAACAGAACAAAAA
GATGCTCTGTGTAAATTCCTTCCTGTAGGGCACACTGCAGGATTTCCATGTAGATAG
AAGAACTATAGGGCCTAGTACAGAAGGTGCACACAAATGTTGGCAAAGTCAAAACC
CCATGAATTAAAACCTACTGGAATTTGGTTTTTAGGAGTTTGGTAATTAGATTATCTC
TTTTGTTATTTTCATTCAGTTATATCCTTTGGCTCAGCTAGCTTTGAAATTGGCTGAT
GAAAAAATATACATAAAAGGGTAAAATTCACACATACAGCAAACAAAAATGCACA
AAGCCTGCTTCGTAACTTTTTTTTCTGGAATTGTTTTTCACTTTGCCTTTTTCTGCCAA
AACAATAATCAAAGAACTCTTGCTTTAACCTATTCCTGTACAAAGACTGTTTTTGAC
CAGATAATCATCTGTTGTGGCATTCTATCTTGTAGGACACTGTATATTGCAAATTGCT
GATTATGGAAGGGGCCAGTTGCTGTTTTTTCATGCAGTGCCCTGGGAGTCTTAAAAG
CAGTGCTTAGCAACATTGGTGATAGCATGTGGCTGGGACCCAGGGCCCTTCCCCACT
CTTCAGCCCCGAGTCATGTGTCTGAGGTGACGGACTGAGACGCATCTGGTCCTGTAA
TTCAGAGAGTGGGCACATCACCAAAGAACTGCATTGCTGTGGTCACTGTTTCTTCAA
GTACACACTGACTCTGCTACTTTAGGATAAATATATTTTACTCAGAACTCTGAATTTC
ACAGTATACTTACTAAACTAAGTAAAAATGATACTTAAAATACTTATTTTACTTTCT
AGACCTAGGCTAGATGTTTTAAGCTACAGCTCTAGTTCATTGTGATATTTATAATTTG
AAAGCTATGAGAATAGATGTGTGGGTGAAGCCATAGAACATATTTGCTTGAAATTCT
TGAGCAGGGATCTTATAAAGGGCCAGAAATAAGATGTGTGGTTCACATAGATAGTG
AGCGTAACATCTGTATTAAACATAGGAGAGAAGTTTATAAAGGGCATTGGCAATAA
ACTCTTTGTTGCAGCTGTTTTCCAAGCAGTGTAAATACTTTTTCCTGTGATTATGTAT
AGCCTTGGAATGGCACCTTTTAACTAACCCATATGTGTTTGGTTTCAATGGTTTTTTA
TATTCAGATGTATATATGGTGCTCACTTTAGGATCAGCAGTGTTGACCATTTATGCTG
CATAGCTGTATTATAGCCTTATTAGTTGTGTGGTTGACCCTTGGGGTATACAAATGTC
AGTCTGAGTGGTGTCTTACTCCTTTGTTTATAAGTGAATGATTGTGCATGTTTTGTAT
GTCATAGTATGTCGTCACATAAAAGGGAGGGAGCGAAAAACCATTACATTAAGATA
ATATTGGACCAAACTACTTACTTGCTCTAAACAGTTACTTGTACCCCTTAACCTGTCT
TCAAAAGTTGCATATAGTTACAGTAGTGTATAAATTAAATATTGTGGAAAAACAGTC
TTGTATTTTTCTGTATGTGTGTATATATATATAATTATGTACTTCTGGCAATTCTATCT
GTATTTAAAGATGTGACAATCTTGACACCAATTTTAAGAATAGCTGTGAGACCGAAT
TAAAGATAATCCCTACCAAGTGAAAATTGATGTGTGTTAAGAGGGTACAGAATTAT
CAACTGATTTGGTCAGTTGCTTCCAATGCTGGTTGATTTCCCTCATTGTGTAAACATT
GACAGGTATGTGACAAATGGGAAAAAAAATCCAAATAATAAAGTGACATATTGGTG
TTCAGCAATATAAA.

By “SNF-RELATED KINASE (Snrk) polypeptide” is meant a polypeptide or fragment thereof having at least about 85% amino acid identity to UniProt Accession No. Q9NRH2, and having kinase activity. An exemplary Snrk polypeptide sequence is provided below:

(SEQ ID NO: 10)
MAGFKRGYDGKIAGLYDLDKTLGRGHFAVVKLARHVFTGEKVAVKVIDK
TKLDTLATGHLFQEVRCMKLVQHPNIVRLYEVIDTQTKLYLILELGDGG
DMFDYIMKHEEGLNEDLAKKYFAQIVHAISYCHKLHVVHRDLKPENVVF
FEKQGLVKLTDFGFSNKFQPGKKLTTSCGSLAYSAPEILLGDEYDAPAV
DIWSLGVILFMLVCGQPPFQEANDSETLTMIMDCKYTVPSHVSKECKDL
ITRMLQRDPKRRASLEEIENHPWLQGVDPSPATKYNIPLVSYKNLSEEE
HNSIIQRMVLGDIADRDAIVEALETNRYNHITATYFLLAERILREKQEK
EIQTRSASPSNIKAQFRQSWPTKIDVPQDLEDDLTATPLSHATVPQSPA
RAADSVLNGHRSKGLCDSAKKDDLPELAGPALSTVPPASLKPTASGRKC
LFRVEEDEEEDEEDKKPMSLSTQVVLRRKPSVTNRLTSRKSAPVLNQIF
EEGESDDEFDMDENLPPKLSRLKMNIASPGTVHKRYHRRKSQGRGSSCS
SSETSDDDSESRRRLDKDSGFTYSWHRRDSSEGPPGSEGDGGGQSKPSN
ASGGVDKASPSENNAGGGSPSSGSGGNPTNTSGTTRRCAGPSNSMQLAS
RSAGELVESLKLMSLCLGSQLHGSTKYIIDPQNGLSFSSVKVQEKSTWK
MCISSTGNAGQVPAVGGIKFFSDHMADTTTELERIKSKNLKNNVLQLPL
CEKTISVNIQRNPKEGLLCASSPASCCHVI.

By “SPEG COMPLEX LOCUS (Speg) polynucleotide” is meant a polynucleotide that encodes a Speg polypeptide or a fragment thereof. An exemplary Speg polynucleotide sequence is provided at NCBI Reference Sequence No: XM_011510483.2.

By “SPEG COMPLEX LOCUS (Speg) polypeptide” is meant a polypeptide or fragment thereof having at least about 85% amino acid identity to UniProt Accession No. Q15772, and having kinase activity.

By “SPHINGOSINE KINASE 1 (Sphk1) polynucleotide” is meant a polynucleotide that encodes a Sphk1 polypeptide or a fragment thereof. An exemplary Sphk1 polynucleotide sequence is provided at NCBI Reference Sequence No: NM_021972.4.

By “SPHINGOSINE KINASE 1 (Sphk1) polypeptide” is meant a polypeptide or fragment thereof having at least about 85% amino acid identity to UniProt Accession No. Q9NYA1, and having kinase activity.

By “SPHINGOSINE KINASE 2 (Sphk2) polynucleotide” is meant a polynucleotide that encodes a Sphk2 polypeptide or a fragment thereof. An exemplary Sphk2 polynucleotide sequence is provided at NCBI Reference Sequence No: NM_020126.5.

By “SPHINGOSINE KINASE 2 (Sphk2) polypeptide” is meant a polypeptide or fragment thereof having at least about 85% amino acid identity to UniProt Accession No. Q9NRA0, and having kinase activity.

By “SPHINGOSINE-1-PHOSPHATE PHOSPHATASE 1 (Sgpp1) polynucleotide” is meant a polynucleotide that encodes a Sgpp1 polypeptide or a fragment thereof. An exemplary Sgpp1polynucleotide sequence is provided at NCBI Reference Sequence No: XM_017021678.2.

By “SPHINGOSINE-1-PHOSPHATE PHOSPHATASE 1 (Sgpp1) polypeptide” is meant a polypeptide or fragment thereof having at least about 85% amino acid identity to UniProt Accession No. Q9BX95, and having phosphatase activity.

By “SPT16 HOMOLOG, FACILITATES CHROMATIN REMODELING SUBUNIT (SUPT16) polynucleotide” is meant a polynucleotide that encodes a SUPT16 polypeptide or a fragment thereof. An exemplary SUPT16 polynucleotide sequence is provided at NCBI Reference Sequence No: NM_007192.4.

By “SRY-BOX 15 (Sox15) polynucleotide” is meant a polynucleotide that encodes a Sox15 polypeptide or a fragment thereof having at least about 85% amino acid identity to NCBI Reference Sequence No: NM_006942.2.

By “SRY-BOX 15 (Sox15) polypeptide” is meant a polypeptide or fragment thereof having at least about 85% amino acid identity to UniProt Accession No. O60248, and having DNA binding and/or transcriptional regulatory activity.

By “SRY-BOX 7 (Sox7) polynucleotide” is meant a polynucleotide that encodes a Sox7 polypeptide or a fragment thereof having at least about 85% amino acid identity to NCBI Reference Sequence No: NM_031439.4.

By “SRY-BOX 7 (Sox7) polypeptide” is meant a polypeptide or fragment thereof having at least about 85% amino acid identity to UniProt Accession No. Q9BT81, and having transcriptional regulatory activity.

By “STE20-LIKE PROTEIN KINASE (Slk) polynucleotide” is meant a polynucleotide that encodes a Slk polypeptide or a fragment thereof. An exemplary Slk polynucleotide sequence is provided at NCBI Reference Sequence No: NM_001304743.2.

By “STE20-LIKE PROTEIN KINASE (Slk) polypeptide” is meant a polypeptide or fragment thereof having at least about 85% amino acid identity to UniProt Accession No. Q9H2G2, and having kinase activity.

By “STE20-RELATED KINASE ADAPTOR ALPHA (Stradu) polynucleotide” is meant a polynucleotide that encodes a Stradα polypeptide or a fragment thereof having at least about 85% amino acid identity to NCBI Reference Sequence No: NM_001363789.1.

By “STE20-RELATED KINASE ADAPTOR ALPHA (Stradu) polypeptide” is meant a polypeptide or fragment thereof having at least about 85% amino acid identity to UniProt Accession No. Q7RTN6, and kinase activity.

By “structure-specific recognition protein 1 (SSRP1) polynucleotide” is meant a polynucleotide that encodes a SSRP1 polypeptide or a fragment thereof. In one embodiment, the polynucleotide sequence is provided at NCBI Reference Sequence No: XM_024448666.1.

By “structure-specific recognition protein 1 (SSRP1) polypeptide” is meant a polypeptide or fragment thereof having at least about 85% amino acid identity to UniProt Accession No. Q08945, and which binds an antibody that specifically binds a SSRP1 polypeptide.

By “SUPT16 polypeptide” is meant a polypeptide or fragment thereof having at least about 85% amino acid identity to UniProt Accession No. Q9Y5B9, and which binds an antibody that specifically binds a SUPT16 polypeptide.

By “TESTIS-SPECIFIC PROTEIN KINASE 1 (Tesk1) polynucleotide” is meant a polynucleotide that encodes a Tesk1 polypeptide or a fragment thereof. An exemplary Tesk1 polynucleotide sequence is provided at NCBI Reference Sequence No: NM_006285.3.

By “TESTIS-SPECIFIC PROTEIN KINASE 1 (Tesk1) polypeptide” is meant a polypeptide or fragment thereof having at least about 85% amino acid identity to UniProt Accession No. Q15569, and having kinase activity.

By “TESTIS-SPECIFIC SERINE/THREONINE KINASE 4 (Tssk4) polynucleotide” is meant a polynucleotide that encodes a Tssk4 polypeptide or a fragment thereof. An exemplary Tssk4 polynucleotide sequence is provided at NCBI Reference Sequence No: XM_024449544.1.

By “TESTIS-SPECIFIC SERINE/THREONINE KINASE 4 (Tssk4) polypeptide” is meant a polypeptide or fragment thereof having at least about 85% amino acid identity to UniProt Accession No. Q6SA08, and having kinase activity.

By “THYMOPOIETIN (Tmpo) polynucleotide” is meant a polynucleotide that encodes a Tmpo polypeptide or a fragment thereof having at least about 85% amino acid identity to NCBI Reference Sequence No: XM_005269132.4.

By “THYMOPOIETIN (Tmpo) polypeptide” is meant a polypeptide or fragment thereof having at least about 85% amino acid identity to UniProt Accession No. P42166, and which binds an antibody that specifically binds a Tmpo polypeptide.

By “TIA1 CYTOTOXIC GRANULE-ASSOCIATED RNA-BINDING PROTEIN-LIKE 1 (Tial1) polynucleotide” is meant a polynucleotide that encodes a Tial1 polypeptide or a fragment thereof having at least about 85% amino acid identity to NCBI Reference Sequence No: NM 001323967.2.

By “TIA1 CYTOTOXIC GRANULE-ASSOCIATED RNA-BINDING PROTEIN-LIKE 1 (Tial1) polypeptide” is meant a polypeptide or fragment thereof having at least about 85% amino acid identity to UniProt Accession No. Q01085, and having transcriptional regulatory activity.

By “transcription factor 24 (Tcf24) polynucleotide” is meant a polynucleotide that encodes a Tcf24 polypeptide or a fragment thereof having at least about 85% amino acid identity to NCBI Reference Sequence No: NM_001193502.1.

By “transcription factor 24 (Tcf24) polypeptide” is meant a polypeptide or fragment thereof having at least about 85% amino acid identity to UniProt Accession No. Q7RTU0, and which binds DNA and/or regulates transcription.

By “TRANSCRIPTION FACTOR 3 (Tcf3) polynucleotide” is meant a polynucleotide that encodes a Tcf3 polypeptide or a fragment thereof. An exemplary Tcf3 polynucleotide sequence is provided at NCBI Reference Sequence No: XM_024451677.1.

By “TRANSCRIPTION FACTOR 3 (Tcf3) polypeptide” is meant a polypeptide or fragment thereof having at least about 85% amino acid identity to UniProt Accession No. P15923, and which binds DNA and regulates transcription.

By “TRANSFORMING GROWTH FACTOR-BETA-INDUCED FACTOR (Tgif1) polynucleotide” is meant a polynucleotide that encodes a Tgif1 polypeptide or a fragment thereof having at least about 85% amino acid identity to NCBI Reference Sequence No: NM_001278684.2.

By “TRANSFORMING GROWTH FACTOR-BETA-INDUCED FACTOR (Tgif1) polypeptide” is meant a polypeptide or fragment thereof having at least about 85% amino acid identity to UniProt Accession No. Q15583, and having transcription regulatory activity.

By “TYROSINE 3-MONOOXYGENASE/TRYPTOPHAN 5-MONOOXYGENASE ACTIVATION PROTEIN, ZETA ISOFORM (Ywhaz) polynucleotide” is meant a polynucleotide that encodes a Ywhaz polypeptide or a fragment thereof. An exemplary Ywhaz polynucleotide sequence is provided at NCBI Reference Sequence No: NM_001135700.2.

By “TYROSINE 3-MONOOXYGENASE/TRYPTOPHAN 5-MONOOXYGENASE ACTIVATION PROTEIN, ZETA ISOFORM (Ywhaz) polypeptide” is meant a polypeptide or fragment thereof having at least about 85% amino acid identity to UniProt Accession No. P63104, and which binds an antibody that specifically binds a Ywhaz polypeptide.

By “TYROSINE KINASE WITH IMMUNOGLOBULIN AND EGF FACTOR HOMOLOGY DOMAINS 1 (TIE1) polynucleotide” is meant a polynucleotide that encodes a TIE1 polypeptide or a fragment thereof. An exemplary TIE1 polynucleotide sequence is provided at NCBI Reference Sequence No: XM_006710869.1.

By “TYROSINE KINASE WITH IMMUNOGLOBULIN AND EGF FACTOR HOMOLOGY DOMAINS 1 (TIE1) polypeptide” is meant a polypeptide or fragment thereof having at least about 85% amino acid identity to UniProt Accession No. P35590, and having kinase activity.

By “URIDINE/CYTIDINE KINASE-LIKE 1 (Uckl1) polynucleotide” is meant a polynucleotide that encodes a Uckl1 polypeptide or a fragment thereof. An exemplary Uckl1polynucleotide sequence is provided at NCBI Reference Sequence No: NM_001353476.2.

By “URIDINE/CYTIDINE KINASE-LIKE 1 (Uckl1) polypeptide” is meant a polypeptide or fragment thereof having at least about 85% amino acid identity to UniProt Accession No. Q9NWZ5, and having kinase activity.

By “VAC14 COMPONENT OF PIKFYVE COMPLEX (Pikfyve) polynucleotide” is meant a polynucleotide that encodes a Pikfyve polypeptide or a fragment thereof. An exemplary Pikfyve polynucleotide sequence is provided at NCBI Reference Sequence No: XM_005256038.4.

By “VAC14 COMPONENT OF PIKFYVE COMPLEX (Pikfyve) polypeptide” is meant a polypeptide or fragment thereof having at least about 85% amino acid identity to UniProt Accession No. Q08AM6, and having kinase activity.

By “Zinc finger protein (Zfp930) polynucleotide” is meant a polynucleotide that encodes a Zfp930 polypeptide or a fragment thereof having at least about 85% amino acid identity to European Nucleotide Archive Accession No.: BAA01477.1.

By “Zinc finger protein (Zfp930) polypeptide” is meant a polypeptide or fragment thereof having at least about 85% amino acid identity to UniProt Accession No. Q62512, and having DNA binding activity.

By “agent” is meant any small molecule chemical compound, polypeptide, nucleic acid molecule, or fragments thereof.

By “ameliorate” is meant decrease, suppress, attenuate, diminish, arrest, or stabilize the development or progression of a disease.

By “alteration” is meant a change (increase or decrease) in the expression levels, structure, or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

“Detect” refers to identifying the presence, absence or amount of the analyte to be detected.

By “disease” is meant any condition or disorder that damages or interferes with the normal function of a cell, tissue, or organ. Examples of diseases include but are not limited to Alzheimer's disease, Parkinson's disease, Huntington's disease, multiple sclerosis (MS), amyotrophic lateral sclerosis (ALS), glaucoma, metachromatic leuokodystrophy, adrenoleukodystophy, lysosomal storage disorders, traumatic brain injury, spinal cord injury, spinal cord crush, and/or optic nerve injury. In some embodiments, the disease is a neurological condition.

By “effective amount” is meant the amount of an agent required to ameliorate the symptoms of a disease relative to an untreated patient. The effective amount of active compound(s) used to practice the present invention for therapeutic treatment of a neurodegenerative disease varies depending upon the manner of administration, the age, body weight, and general health of the subject. Ultimately, the attending physician or veterinarian will decide the appropriate amount and dosage regimen. Such amount is referred to as an “effective” amount.

In some embodiments, an effective amount is the amount of an inhibitory nucleic acid (e.g., siRNA, shRNA, antisense polynucleotide) required to treat a disease or disorder (e.g., neurodegenerative disease, and/or neuronal injury) or symptom thereof, in a subject. In some embodiments, an effective amount is the amount of a vector encoding an inhibitory nucleic acid (e.g., siRNA, shRNA, antisense polynucleotide) required to treat a disease or disorder (e.g., neurodegenerative disease, and/or neuronal injury) or symptom thereof, in a subject. In some embodiments, the inhibitory nucleic acid is a siRNA, shRNA, or antisense polynucleotide. In some embodiments, an effective amount is the amount of a nucleic acid molecule or polynucleotide (e.g., mRNA or DNA), an encoded polypeptide, or a polypeptide (e.g., a gene-editing polypeptide) required to treat a disease or disorder (e.g., neurodegenerative disease, and/or neuronal injury) or symptom thereof, in a subject. In some embodiments, an effective amount is the amount of a polypeptide (e.g., a gene-editing polypeptide) required to treat a disease or disorder (e.g., neurodegenerative disease, and/or neuronal injury) or symptom thereof, in a subject. In various embodiments, the polypeptide is a zinc finger nuclease (ZFN), a Transcription Activator-Like Effector Nuclease (TALEN), or a CRISPR polypeptide (e.g., Cas9, saCas9, or spCas9). In some embodiments, the nucleic acid molecule contains, consists of, or encodes single-guide RNA. In some embodiments, an effective amount is the amount of a vector (e.g., an adeno-associated virus (AAV) vector) required to treat a disease or disorder (e.g., neurodegenerative disease, and/or neuronal injury) or symptom thereof, in a subject.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, “heterologous” is used to refer to a gene, polynucleotide, or polypeptide experimentally put into a cell or virus particle that does not normally comprise that polynucleotide or polypeptide. In various embodiments “heterologous” is used to refer to a sequence derived from a different cell or virus from that virus or cell into which the sequence has been introduced.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 25%, 50%, 75%, or 100%.

By “inhibitory nucleic acid” is meant a double-stranded RNA, siRNA, shRNA, or antisense RNA, or a portion thereof, or a mimetic thereof, that when administered to a mammalian cell results in a decrease in the expression of a target gene. In various embodiments, the expression of the target gene is decreased by 10%, 25%, 50%, 75%, or even 90-100%. In some embodiments, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany the material in the material's original source or surroundings. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using techniques in analytical chemistry or microscopy, for example, polyacrylamide gel electrophoresis, mass spectroscopy, electron microscopy, or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “knockout” is meant an alteration that renders a gene inoperative. In some embodiments, the alteration is a nonsense mutation, an indel mutation, or a deletion of the gene.

By “marker” is meant any analyte, protein or polynucleotide having an alteration in expression level or activity that is associated with a disease or disorder.

By “neuroprotective” is meant increase survival of a neuron at risk of cell death. In various embodiments, a neuroprotective agent effects an increase in neuron survival of about or of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, or 500%.

In some embodiments, the neuroprotective agent knocks out a target gene or inhibits a biological activity of a molecular target.

By “neuroregenerative” is meant to positively effect regrowth of an axon from a site of injury. In various embodiments, a neuroregenerative agent effects a regrowth of an axon of about or at least about 5 μm, 10 μm, 15 μm, 20 μm, 25 μm, 30 μm, 35 μm, 40 μm, 45 μm, 50 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 1,000 μm, 1,500 μm, 2000 μm, 2,500 μm, or 5,000 μm. In some embodiments, the neuroregenerative agent knocks out a target gene or inhibits a biological activity of a molecular target.

The term “nucleotide molecule”, “polynucleotide”, or “nucleic acid sequence” are used interchangeably to refer to a molecule comprising a polymer of nucleobases. In some embodiments, a polynucleotide comprises RNA, or DNA. In various embodiments, the nucleotide molecule or polynucleotide comprises modified nucleotides (e.g., locked nucleic acids (LNA)). In some embodiments, the nucleotide molecule or polynucleotide comprises RNA and DNA. The sugar backbone of the nucleotide molecule is non-limiting and may comprise ribose, deoxyribose, or various other suitable sugars. In some embodiments, the nucleic acid molecule comprises at least two nucleotides covalently linked together. In some embodiments, the nucleic acid molecule of the present invention is single-stranded. In some embodiments, the nucleic acid molecule is double stranded. In some embodiments, the nucleic acid molecule is triple-stranded. In some embodiments, the nucleic acid molecule comprises phosphodiester bonds. In some embodiments, the nucleic acid molecule comprises a single-stranded or double-stranded deoxyribonucleic acid (DNA) or a single-stranded or double-stranded ribonucleic acid (RNA). In some embodiments, the nucleic acid molecule comprises a nucleic acid analog. In some embodiments, the nucleic acid analog has a backbone, comprising a bond other than and/or in addition to a phosphodiester bond, such as, by non-limiting example, phosphoramide, phosphorothioate, phosphorodithioate or O-methylphophoroamidite linkage. In some embodiments, the nucleic acid analog is selected from a nucleic acid analog with a backbone selected from a positive backbone; a non-ionic backbone and a non-ribose backbone. In some embodiments, the nucleic acid molecule contains one or more carbocyclic sugars. In some embodiments, the nucleic acid molecule comprises modifications of its ribose-phosphate backbone. In some embodiments, these modifications are performed to facilitate the addition of additional moieties, such as labels. In some embodiments, these modifications are performed to increase the stability and half-life of such molecules in physiological “Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral methyl phosphonates, 2-O-methyl ribonucleotides, and peptide-nucleic acids (PNAs).

Unless otherwise indicated, a particular nucleic acid sequence also implicitly encompasses conservatively modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al, Nucleic Acid Res, 19:081, 1991; Ohtsuka et al., J. Biol. Chem., 260:2600-2608, 1985; Rossolini et al., Mol. Cell Probes, 8:91-98, 1994). The term nucleic acid can be used interchangeably with gene, cDNA, mRNA, oligonucleotide, and polynucleotide. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

As used herein, “obtaining” as in “obtaining an agent” includes synthesizing, purchasing, or otherwise acquiring the agent.

As used herein “pharmaceutically acceptable” means that which is useful in preparing a pharmaceutical composition that is generally safe and non-toxic to a subject. In various embodiments, pharmaceutically acceptable components of a composition include substances acceptable for veterinary use and/or human pharmaceutical use. In some embodiments pharmaceutically-acceptable refers to molecular entities and compositions that do not produce an allergic or similar untoward reaction when administered to a subject.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

As used herein, “proximal” is used to indicate adjacency to or nearly contacting. For example, in various embodiments, an axon or nerve cell body is proximal to a site of injury if the axon or nerve cell body is no more than about 1 mm, 2 mm, 3 mm, 4 mm, 5 mm, 6 mm, 7 mm, 8 mm, 9 mm, 10 mm, 15 mm, 20 mm, 25 mm, 30 mm, 40 mm, or 50 mm from a site of injury or from a center of a site of spinal cord injury, as measured along the injured spinal cord.

As used herein “recombinant” is used to refer to molecules or polypeptides formed by laboratory methods of genetic recombination to bring together genetic material from multiple sources to create sequences not otherwise found in nature.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “retrograde infection” is meant spread of a virus following infection at an axon of a neuron to the cell body of the neuron.

By “retrograde degeneration” is meant the dieback of a neuron toward the cell body of the neuron from a site of damage in an axon of the neuron.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The terms “transduction” and “transfection” are used interchangeably and refers to the process by which a viral vector, nucleic acid molecule, or a portion thereof is introduced into a cell.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “vector” refers to an agent that contains or carries modified genetic material and can be used to introduce heterologous genes to a host cell. The vector can be, for example, a virus vector, a virus-like particle, a non-viral vector, a plasmid, or a cosmid.

The vector can be a virus particle. The vector can be a nanoparticle (e.g., an inorganic nanoparticle and/or a protein/peptide-based nanoparticle), a carbon nanotube, a liposome, or a nanoscale polymeric material. Additional non-limiting examples of non-viral vectors suitable for use in the invention of the disclosure are those described in Yin, H. et al., “Non-viral vectors for gene-based therapy”, Nature Reviews Genetics, 15:541-555 (2014), the entirety of which is incorporated herein by reference for all purposes. A vector as used herein can be composed of either DNA or RNA. In some embodiments, a vector is composed of DNA. The vector can be an expression vector. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a schematic of the clustered regularly interspaced short palindromic repeats (CRISPR)/Cas9 screen experimental design using the mouse optic nerve crush (ONC) model. Briefly, Rosa-Lox-Stop-Lox-Cas9-EGFP knock-in (LSL-Cas9) mice were intravitreally injected with adeno-associated virus serotype 2 (AAV2)-Cre and AAV2-sgRNA (for target genes) 2 weeks before optic nerve crush (ONC). Two weeks after optic nerve crush (ONC), Cholera Toxin Subunit B (CTB)-555 was intravitreally injected before retinal ganglion cell (RGC) survival and axon regeneration were analyzed.

FIGS. 2A-2D are images and bar graphs demonstrating that clustered regularly interspaced short palindromic repeats (CRISPR)/Cas9-mediated deletion of Pten promotes retinal ganglion cell (RGC) survival and axon regeneration after optic nerve crush (ONC) injury: FIG. 2A provides bar graphs showing quantification of retinal ganglion cell (RGC) survival in control (AAV2-Cre only) and Pten sgRNA (+AAV2-Cre) retinas 2 weeks after optic nerve crush (ONC). FIG. 2B presents representative images of retinal wholemounts immunostained with the retinal ganglion cell (RGC) marker RNA-binding protein with multiple splicing (RBMPS). FIG. 2C presents bar graphs providing quantification of the density of retinal ganglion cell (RGC) axon regeneration of control (AAV2-Cre only) and Pten sgRNA (+AAV2-Cre) optic nerves 2 weeks after optic nerve crush (ONC). FIG. 2D provides representative confocal image stacks of tissue cleared optic nerves showing regenerated retinal ganglion cell (RGC) axons labeled by CTB-555 intraocular injection two weeks after optic nerve crush (ONC). n=3 retinas or optic nerves per group. Scale bar=200 μm. **** p<0.0001.

FIG. 3 provides a schematic of a neuron and lists methods for enhancing intrinsic axon regenerative ability. See He, Z. and Jin, Y., “Intrinsic Control of Axon Regeneration”, Neuron, 90:P 437-451 (2016), DOI: 10.1016/j.neuron.2016.04.022.

FIG. 4 is a schematic of a clustered regularly interspaced short palindromic repeats (CRISPR)/Cas9 system for sequence-specific genome editing. To delete target genes, a chimeric single guide RNA (sgRNA) is generated to direct the Cas9 endonuclease to a specific genomic location (˜20 nucleotide spacer). Cas9-induced double stranded DNA breaks (upstream of protospacer adjacent motif (PAM)) are mostly repaired via error-prone non-homologous end joining (NHEJ) pathway.

FIG. 5 is a schematic and images summarizing the forward genetic CRISPR screen design. Briefly, plasmid and AAV libraries were prepared. LSL-Cas9 mice were intravitreally injected with AAV2-Cre and AAV2-sgRNA (for target genes) 2 weeks before ONC. Two weeks after ONC, CTB-555 was intravitreally injected before RGC survival and axon regeneration phenotypes were analyzed.

FIGS. 6A-6D provide bar graphs and images demonstrating that the CRISPR screen uncovered new pro-survival and pro-regenerative gene targets. FIGS. 6A and 6B are a bar graph and images presenting data relating to gene targets promoting retinal ganglion cell (RGC) survival when knocked out. 25 of 57 hit genes were shown to significantly promote retinal ganglion cell survival (RGC) when knocked out, compared to control. FIGS. 6C and 6D are a bar graph and images presenting data relating to gene targets promoting retinal ganglion cell (RGC) regeneration when knocked out. 5 of 32 hit genes were shown to significantly promote RGC axon regeneration when knocked out, compared to control. In some embodiments, the target genes are kinases (e.g., darkest grey bars in FIGS. 6A and 6C) or phosphatases (e.g., medium-shade grey bars on the right or bottom portion of the bar graph in FIGS. 6A and 6C, respectively). Scale bar, 200 μm.

FIGS. 7A-7C are a bar graph and images. FIGS. 7A and 7B are a bar graph and images demonstrating that CRISPR deletion of Map2k7 completely protects RGCs from cell death after optic nerve crush injury and prevents retrograde (‘die-back’) degeneration of CNS neurons, when compared to control and intact (no ONC). FIG. 7C presents images demonstrating that CRISPR deletion of MAPK8IP3 protects RGCs from cell death after optic nerve crush injury. Scale bar, 200 and 1000 μm.

FIGS. 8A-8S present a bar graph and images relating to significant retinal ganglion cell (RGC) axon regeneration resulting from knock-out of indicated genes. FIG. 8A is a bar graph. FIGS. 8B-8S are images. Scale bar, 0.5 mm.

FIGS. 9A-9B are images and a bar graph relating to retinal ganglion cell (RGC) survival resulting from knock-out of indicated genes. FIG. 9A demonstrates significantly improved survival after optical nerve crush (ONC) injury by CRISPR deletion of several target genes, compared to control. Scale bar, 50 μm. FIG. 9B is a bar graph. In some embodiments, the target genes are transcription factors.

### DETAILED DESCRIPTION OF THE INVENTION

The invention features compositions and methods that are useful for neuroprotection and/or neuroregeneration of damaged neurons. In some embodiments, the damaged neurons are undergoing neurodegeneration. In various embodiments, the compositions and methods of the present disclosure are used to treat neurodegenerative diseases and central nervous system (CNS) traumas/injuries. The methods in various embodiments include reducing or eliminating activity or expression of a target gene(s) and/or a polypeptide(s) expressed by a target gene(s) in a neuron. In various embodiments, the neurons are damaged or degenerating due to a trauma/injury and/or a neurodegenerative disease.

The invention of the disclosure is based, at least in part, upon the identification of genes whose knockout significantly increases neuronal survival and/or neuronal regeneration. As described in the Examples provided herein an unbiased CRISPR-Cas9 forward genetic screen of over 2,000 genes was used to identify genes whose knockout increased neuronal survival and/or neuronal regeneration in the mouse optic nerve crush (ONC) model. The mouse optic nerve crush (ONC) model is a model of mammalian CNS neuron degeneration, in which injured CNS neurons—called retinal ganglion cells (RGCs)—reproducibly undergo neurodegeneration and fail to regenerate spontaneously. Accordingly, the invention provides in various embodiments new targets for neuroprotection and/or neurodegeneration therapeutic interventions in neurodegenerative disorders.

### Inhibitory Nucleic Acid Therapy

The inhibition of any one of the following genes resulted in increased survival, reduction in cell death, or protection from neuron degeneration: ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz. In embodiments, inhibition of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or all of the foregoing genes resulted in increased neuron survival, reduction in neuron death, or protection from neuron degeneration.

The inhibition of any one of the following genes results in an increase in axon regrowth or neuroregeneration: Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930. In embodiments, inhibition of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the foregoing genes resulted in neuroregeneration (e.g., axon regrowth). Thus, provided herein are inhibitory nucleic acid molecules (e.g., antisense, siRNA, shRNA), that target such genes. Such nucleic acid molecules can be delivered to cells of a subject having a neurodegenerative disease or damaged or degenerating neurons. The nucleic acid molecules are delivered to the cells of a subject in a form in which they can be taken up so that therapeutically effective levels of the inhibitory nucleic acid molecules are introduced.

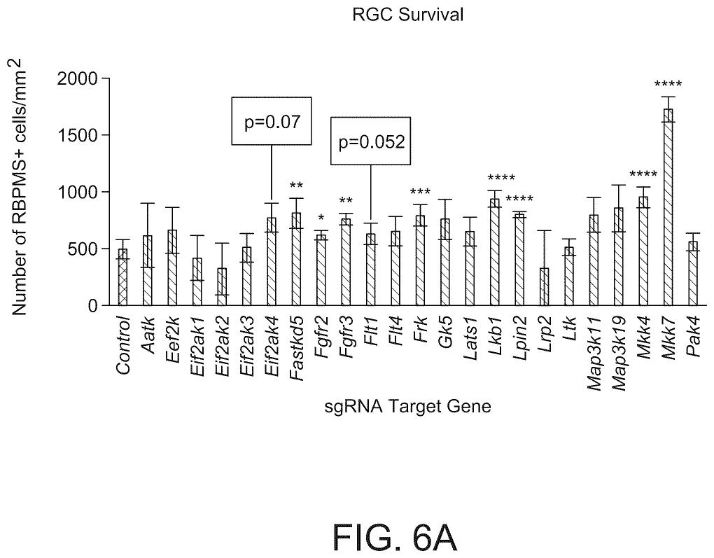

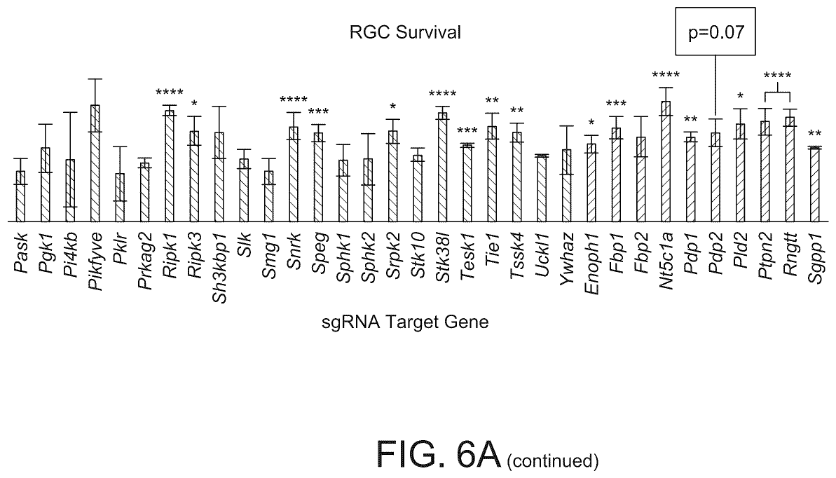

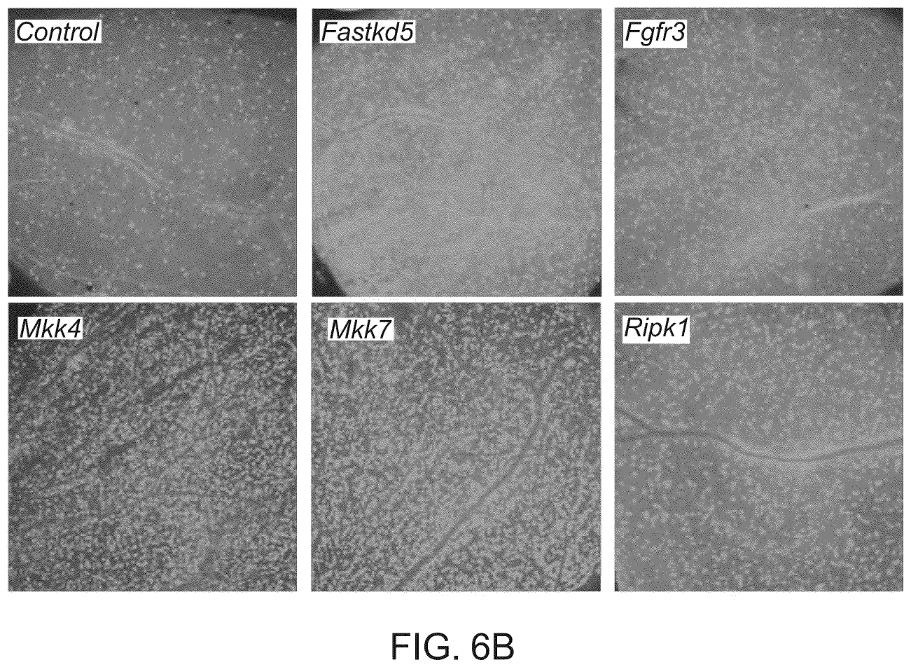

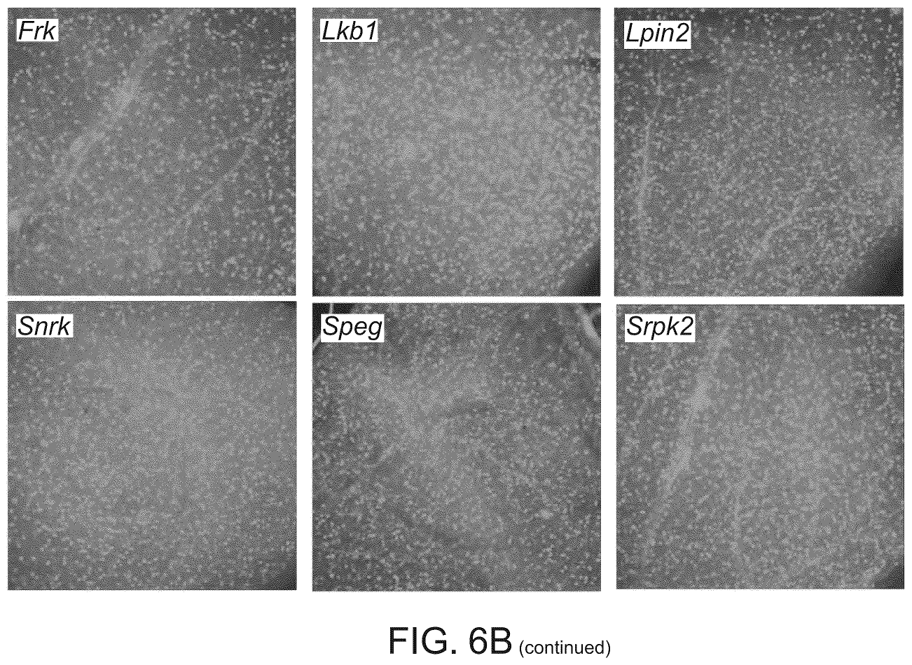

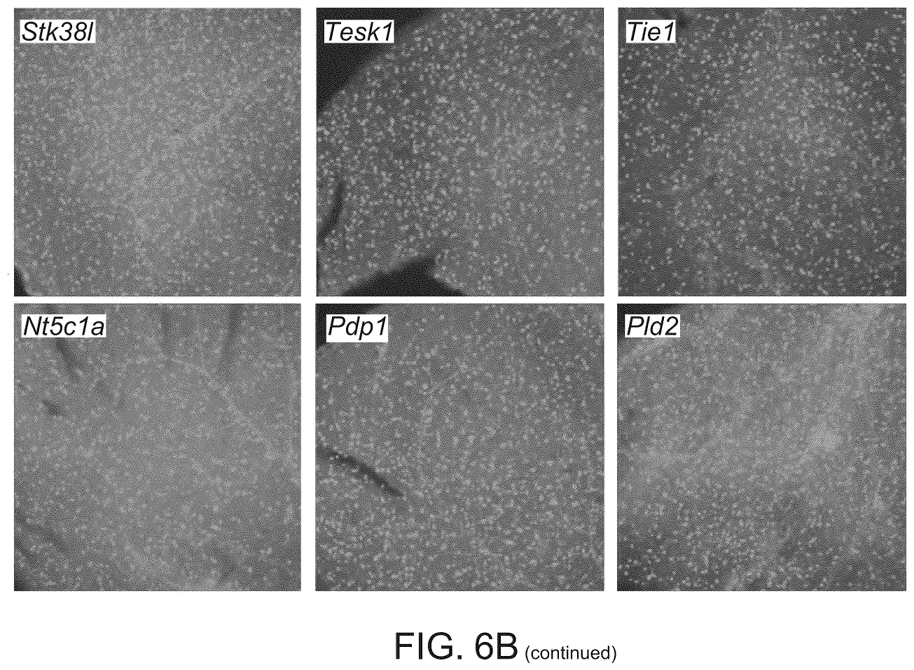

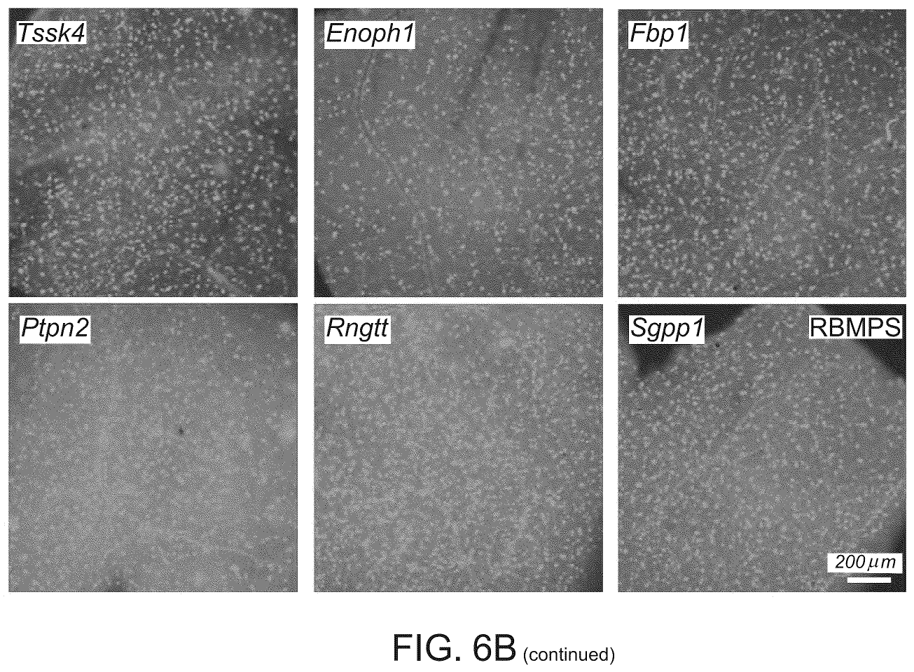

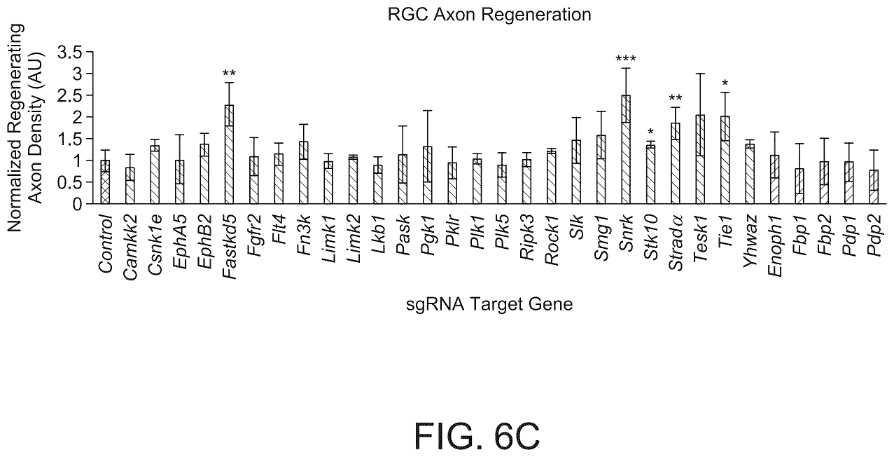

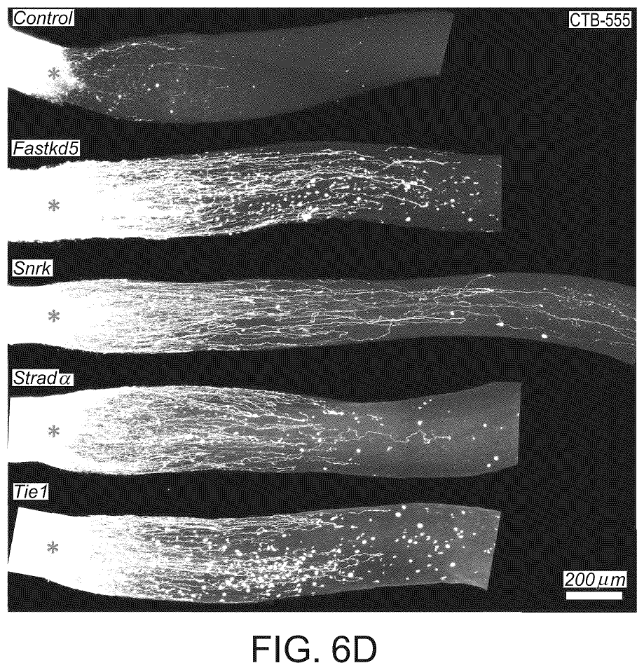

In some embodiments, the target gene is selected from one or more of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz. In some embodiments, more than one target gene is selected. In some embodiments at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the foregoing genes are selected. In some embodiments, the target gene is selected from one or more of ATF3, ATF4, CEBPG, CHOP, Lkb1, Lpin2, Mkk4, Mkk7, Nt5c1a, SSRP1, and SUPT16. In some embodiments, the target gene is selected from one or more of MAPK8IP3, Mkk7, and Snrk. In some embodiments, the target gene is selected from one or more of the 1, 2, 3, 4, 5, 6, 7, 8, 9, 10, 15, 20, 25, 30, 35, 40, 45, or 50 genes associated with the highest number of RBPMS+ cells/mm² in FIG. 6A. In some embodiments, the target genes selected comprise MAP2K7 and MAP2K4. In some embodiments, the target gene comprises Lkb1.

In some embodiments, the target gene is selected from one or more of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930. In some embodiments, more than one target gene is selected. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the foregoing genes are selected. In some embodiments, the target gene is selected from one or more of Fastkd5, Lhx6, Pawr, Rbbp7, Snrk, Stk10, Stradα, Tcf3, Tgif1, and Tie1. In some embodiments, the target gene is Snrk. In some embodiments, the target gene is selected from one or more of the 1, 2, 3, 4, 5, 6, 7, 8, 9, 10, 15, 20, 25, or 30 genes associated with the highest normalized regenerating axon density (AU) in FIG. 6C.

In some embodiments where more than one target gene is selected, a synergistic regenerative or cell (e.g., a neuron) survival effect can be achieved by selecting at least one target gene from ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz and selecting at least one other distinct target gene from Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930. In some embodiments, a synergistic effect can be effected by selecting one target gene that is associated with an increase in cell survival (i.e., neuroprotection) and another target gene that is associated with an increase in cell growth (i.e., neuroregeneration).

In one embodiment, the invention provides inhibitory nucleic acid molecules, such as antisense nucleic acid molecules, that decrease the expression of at least one of the above listed target genes. Inhibitory nucleic acid molecules are essentially nucleobase oligomers that may be employed to decrease the expression of a target nucleic acid sequence, such as a nucleic acid sequence that encodes a target gene. The inhibitory nucleic acid molecules provided by the invention include any nucleic acid molecule sufficient to decrease the expression of a nucleic acid molecule of a target gene by about or at least about 5%, 10%, 15%, 20%, 25%, 30%, 35%, 40%, 45%, 50%, 55%, 60%, 65%, 70%, 75%, 80%, 85%, 90%, 95%, or 100%. The inhibitory nucleic acid molecules provided by the invention in various embodiments include any nucleic acid molecule sufficient to decrease the expression of a nucleic acid molecule of a target gene by at least about 5-10%, at least about 25%-50%, at least about 50%-75%, or even by as much as about 75%-100%. Each of the nucleic acid sequences provided herein may be used, for example, in the discovery and development of therapeutic antisense nucleic acid molecules to decrease the expression of a polypeptide or polynucleotide encoded by a target gene. If desired, antisense nucleic acid molecules that target one or more target genes are administered in combination, such that the coordinated reduction in the expression of two or more target genes is achieved.

The invention is not limited to antisense nucleic acid molecules but encompasses virtually any single-stranded or double-stranded nucleic acid molecule that decreases expression of target genes or its encoded RNA/protein products. The invention further provides catalytic RNA molecules or ribozymes. Such catalytic RNA molecules can be used to inhibit expression of a target gene (e.g., Mkk7, MAPK81P3, Map2K4, or Snrk) in vivo.

The inclusion of ribozyme sequences within an antisense RNA confers RNA-cleaving activity upon the molecule, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference. In various embodiments of this invention, the catalytic nucleic acid molecule is formed in a hammerhead or hairpin motif. Examples of such hammerhead motifs are described by Rossi et al., Nucleic Acids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 7/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

In another approach, the inhibitory nucleic acid molecule is a double-stranded nucleic acid molecule used for RNA interference (RNAi)-mediated knock-down of the expression of a target gene. siRNAs are also useful for the inhibition of gene expression (e.g., through targeting mRNAs encoded by a target gene for degradation). See, for example, Nakamoto et al., Hum Mol Genet, 2005. Desirably, the siRNA is designed such that it provides for the cleavage of a target mRNA encoded by a target gene of the invention. In one embodiment, a double-stranded RNA (dsRNA) molecule is made that includes between eight and twenty-five (e.g., 8, 10, 12, 15, 16, 17, 18, 19, 20, 21, 22, 23, 24, 25) consecutive nucleobases of a nucleobase oligomer of the invention. The dsRNA can be two complementary strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. Double stranded RNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference. An inhibitory nucleic acid molecule that “corresponds” to a target gene comprises at least a fragment of the double-stranded gene, such that each strand of the double-stranded inhibitory nucleic acid molecule is capable of binding to the complementary strand of the target gene. The inhibitory nucleic acid molecule need not have perfect correspondence or need not be perfectly complementary to the reference sequence. In one embodiment, an siRNA has at least about 85%, 90%, 95%, 96%, 97%, 98%, or even 99% sequence identity with the target nucleic acid. For example, a 19 base pair duplex having 1-2 base pair mismatch is considered useful in the methods of the invention. In other embodiments, the nucleobase sequence of the inhibitory nucleic acid molecule exhibits 1, 2, 3, 4, 5 or more mismatches.

Inhibitory nucleic acid molecules of the invention also include double stranded nucleic acid “decoys.” Decoy molecules contain a binding site for a transcription factor that is responsible for the deregulated transcription of a gene of interest. The present invention provides decoys that competitively block binding to a regulatory element in a target gene. The competitive inhibition of regulatory element binding by the decoy results in the indirect inhibition of transcription of a target gene. An overview of decoy technology is provided by Suda et al., Endocr. Rev., 1999, 20, 345-357; S. Yla-Hertttuala and J. F. Martin, The Lancet 355, 213-222, 2000). In one therapeutic method, short double-stranded DNA decoy molecules are introduced into cells (e.g., neoplastic cells) of a subject. The decoys are provided in a form that facilitates their entry into target cells of the subject. Having entered a cell, the decoy specifically binds an endogenous transcription factor, thereby competitively inhibiting the transcription factor from binding to an endogenous gene. The decoys are administered in amounts and under conditions whereby binding of the endogenous transcription factor to the endogenous gene is effectively competitively inhibited without significant host toxicity. Depending on the transcription factor, the methods can effect up- or down-regulation of gene expression. The subject compositions comprise the decoy molecules in a context that provides for pharmacokinetics sufficient for effective therapeutic use.

In one embodiment, the inhibitory nucleic acid molecules of the invention are administered systemically in dosages between about 0.01 and 100 mg/kg (e.g., 1, 5, 10, 20, 25, 50, 75, and 100 mg/kg). In other embodiments, the dosage ranges from between about 0.01 and 500 mg/kg/day. The inhibitory nucleic acid molecules can also be introduced into cells by vectors.

### Modified Inhibitory Nucleic Acid Molecules

In some embodiments, a desirable inhibitory nucleic acid molecule is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed to enhance the stability or efficacy of an inhibitory nucleic acid molecule.

Inhibitory nucleic acid molecules include nucleobase oligomers containing modified backbones or non-natural internucleoside linkages. Oligomers having modified backbones include those that retain a phosphorus atom in the backbone and those that do not have a phosphorus atom in the backbone. For the purposes of this specification, modified oligonucleotides that do not have a phosphorus atom in their intemucleoside backbone are also considered to be nucleobase oligomers. Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriesters, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl intemucleoside linkages, mixed heteroatom and alkyl or cycloalkyl intemucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Nucleobase oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleobase oligomer, particularly the 3′ position of the sugar on the 3′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

In other nucleobase oligomers, both the sugar and the internucleoside linkage, i.e., the backbone, are replaced with novel groups. Methods for making and using these nucleobase oligomers are described, for example, in “Peptide Nucleic Acids (PNA): Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In other embodiments, a single stranded modified nucleic acid molecule (e.g., a nucleic acid molecule comprising a phosphorothioate backbone and 2′-O-Me sugar modifications is conjugated to cholesterol. Such conjugated oligomers are known as “antagomirs.” Methods for silencing a target gene in vivo with antagomirs are described, for example, in Krutzfeldt et al., Nature 438: 685-689.

### Inhibitory Polynucleotides for Target Genes

In general, the invention includes any inhibitory nucleic acid sequence (e.g., an inhibitory nucleic acid molecule, such as an antisense molecule, a dsRNA, siRNA, or shRNA) containing at least one strand that hybridizes with a target gene (e.g., Mkk7, MAPK8IP3, Map2K4, and Snrk).

In some embodiments, the target gene is selected from one or more of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz. In some embodiments, more than one target gene is selected. In some embodiments at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the foregoing genes are selected. In some embodiments, the target gene is selected from one or more of ATF3, ATF4, CEBPG, CHOP, Lkb1, Lpin2, Mkk4, Mkk7, Nt5c1a, SSRP1, and SUPT16. In some embodiments, the target gene is selected from one or more of MAPK8IP3, Mkk7, and Snrk. In some embodiments, the target gene is selected from one or more of the 1, 2, 3, 4, 5, 6, 7, 8, 9, 10, 15, 20, 25, 30, 35, 40, 45, or 50 genes associated with the highest number of RBPMS+ cells/mm² in FIG. 6A. In some embodiments, the target genes selected comprise MAP2K7 and MAP2K4. In some embodiments, the target gene comprises Lkb1.

In some embodiments, the target gene is selected from one or more of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930. In some embodiments, more than one target gene is selected. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the foregoing genes are selected. In some embodiments, the target gene is selected from one or more of Fastkd5, Lhx6, Pawr, Rbbp7, Snrk, Stk10, Stradα, Tcf3, Tgif1, and Tie1. In some embodiments, the target gene is Snrk. In some embodiments, the target gene is selected from one or more of the 1, 2, 3, 4, 5, 6, 7, 8, 9, 10, 15, 20, 25, or 30 genes associated with the highest normalized regenerating axon density (AU) in FIG. 6C.

In some embodiments where more than one target gene is selected, a synergistic regenerative or cell (e.g., a neuron) survival effect can be achieved by selecting at least one target gene from ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz and selecting at least one other distinct target gene from Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930. In some embodiments, a synergistic effect can be effected by selecting one target gene that is associated with an increase in cell survival (i.e., neuroprotection) and another target gene that is associated with an increase in cell growth (i.e., neuroregeneration).

The inhibitory nucleic acid molecules of the invention can be between 8 and 45 nucleotides in length. In some embodiments, the inhibitory nucleic acid molecules of the invention comprises 8, 10, 15, 16, 17, 18, 19, 20, 21, 22, 23, 24, 25, 26, 27, 28, 29, 30, 31, 32, 33, 34, 35, or 45 nucleic acid residues or basepairs. In yet other embodiments, the inhibitory nucleic acid molecule is at least about 60%, 65%, 70%, 75%, 80%, 85%, 90%, 95%, 96%, 97%, 98%, 99% complementary to a sequence of a target gene.

### Delivery of Nucleobase Oligomers

Naked oligonucleotides are capable of entering cells and inhibiting the expression of a target gene. Nonetheless, it may be desirable to utilize a formulation that aids in the delivery of an inhibitory nucleic acid molecule or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

Polynucleotides of the present invention can be delivered to a cell using a vector, for example an adeno-associated virus vector (AAV). Interestingly, recombinant adeno-associated virus (rAAV) particles have tissue-specific targeting capabilities, such that a polynucleotide of the rAAV will be delivered specifically to one or more predetermined tissue(s), cell(s), and/or bodily fluids.

More than 30 naturally occurring serotypes of AAV are available and are useful in the particles, vectors, nucleotide molecules, and methods described herein. Many natural variants in the adeno-associated virus (AAV) capsid exist, allowing identification and use of an AAV with properties specifically suited for neural cells as well as other cell types. AAV viruses (i.e., AAV particles) can be engineered by conventional molecular biology techniques, making it possible to optimize these particles for cell specific delivery of the desired nucleic acid sequences, for minimizing immunogenicity, for tuning stability and particle lifetime, for efficient degradation, for accurate delivery to the nucleus, etc.′

The use of adeno-associated viruses (AAVs) is a common mode of exogenous delivery of DNA because AAVs are relatively non-toxic, provide efficient polynucleotide transfer, and can be easily optimized for specific purposes. Among the serotypes of AAVs isolated from human or non-human primates (NHP) and well characterized, human serotype 2 is the first AAV that was developed as a polynucleotide transfer vector. This serotype has been widely used for efficient polynucleotide transfer experiments in different target tissues and animal models. Other AAV serotypes include useful in the vectors and methods of this disclosure include, but are not limited to, AAV1, AAV3, AAV4, AAV5, AAV6, AAV6.2, AAV7, AAV8, AAV9, AAV10, rh.10, rh.39, rh,43, CSP3, and the like.

Transducing viral vectors, such as the AAV vectors described above, are especially suitable for transfer of polynucleotides to somatic cells because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, an inhibitory nucleic acid as described can be cloned into an adeno-associated virus (AAV) or retroviral vector and expression can be driven from its endogenous promoter, from the retroviral long terminal repeat, or from a promoter specific for a target cell type of interest. In some embodiments, the target cell type of interest is a neuron. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). AAV2 and AAV9 have been approved by the Food and Drug Administration of the United States for treatment of a number of neurological conditions. In some embodiments, a viral vector is used to administer a polynucleotide encoding inhibitory nucleic acid molecules that inhibits a target gene of the invention.

Non-viral approaches can also be employed for the introduction of an inhibitory nucleic acid molecule to a cell of a patient requiring treatment. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). In some embodiments, the nucleic acids are administered in combination with a liposome and protamine. Further non-limiting examples of non-viral approaches include use of a nanoparticle (e.g., an inorganic nanoparticle and/or a protein/peptide-based nanoparticle), a carbon nanotube, a liposome, or a nanoscale polymeric material. Additional non-limiting examples of non-viral approaches suitable for use in the invention of the disclosure are those described in Yin, H. et al., “Non-viral vectors for gene-based therapy”, Nature Reviews Genetics, 15:541-555 (2014), the entirety of which is incorporated herein by reference for all purposes.

Polynucleotide transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of polynucleotide encoding inhibitory nucleic acid molecules into the affected tissues of a patient can also be accomplished by transferring a polynucleotide encoding the inhibitory nucleic acid into a cultivatable cell type ex vivo (e.g., an autologous or heterologous primary cell or progeny thereof), after which the cell (or its descendants) are injected into a targeted tissue.

Expression of polynucleotides for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct polynucleotide expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

In some embodiments, the inhibitory nucleic acid molecule is selectively expressed in a neuron. In some other embodiments, the inhibitory nucleic acid molecule is expressed in a neuron using a lentiviral vector. In still other embodiments, the inhibitory nucleic acid molecule is administered intrathecally. In some embodiments, the inhibitory nucleic acid molecule is administered systemically. In some embodiments, the inhibitory nucleic acid molecule is administered locally at a site of injury or neurodegeneration and not systemically. In some embodiments, the inhibitory nucleic acid molecule is administered by a vector targeted to a particular cell type or tissue within a subject. Selective targeting or expression of inhibitory nucleic acid molecules to a neuron is described in, for example, Nielsen et al., J Gene Med. 2009 July; 11(7):559-69. DOI: 10.1002/jgm.1333.

Recombinant Adeno-Associated Viruses (rAAV)

In some embodiments, an inhibitory nucleic acid molecule described herein is administered using an adeno-associated virus. Adeno-associated virus is a small (20-26 nm), icosahedral, and nonenveloped virus. AAV particles contain a single-stranded DNA genome consisting of approximately 4.7 kb. The genome contains three open reading frames (ORFs) encoding for replication proteins (Rep), capsid proteins (Caps), and the assembly activating protein (AAP), and is flanked by two inverted terminal repeats (ITRs). Interestingly, recombinant adeno-associated virus (rAAV) particles have tissue-specific targeting capabilities, such that a heterologous gene of the rAAV will be delivered specifically to one or more predetermined tissue(s), cell(s), or body fluids. A capsid protein encoded by the rAAV facilitates the tissue-specific targeting. In various embodiments, the recombinant adeno-associated virus (rAAV) particles disclosed herein are encoded by any one of the vectors and/or polynucleotides described herein or produced by any one of the methods described herein.

More than 30 naturally occurring serotypes of AAV are available and are useful in the particles, vectors, nucleotide molecules, and methods described herein. Many natural variants in the adeno-associated virus (AAV) capsid exist, allowing identification and use of an AAV with properties specifically suited for neural cells as well as other cell types. AAV viruses (i.e., AAV particles) can be engineered by conventional molecular biology techniques, making it possible to optimize these particles for cell specific delivery of the desired nucleic acid sequences, for minimizing immunogenicity, for tuning stability and particle lifetime, for efficient degradation, for accurate delivery to the nucleus, etc.′

The use of adeno-associated viruses (AAVs) is a common mode of exogenous delivery of polynucleotides because AAVs are relatively non-toxic, provide efficient gene transfer, and can be easily optimized for specific purposes. Among the serotypes of AAVs isolated from human or non-human primates (NHP) and well characterized, human serotype 2 is the first AAV that was developed as a gene transfer vector. This serotype has been widely used for efficient gene transfer experiments in different target tissues and animal models. Other AAV serotypes include useful in the vectors and methods of this disclosure include, but are not limited to, AAV1, AAV3, AAV4, AAV5, AAV6, AAV6.2, AAV7, AAV8, AAV9, AAV10, rh.10, rh.39, rh,43, CSP3, and the like (see, e.g., WO 2005/033321 and U.S. Pat. No. 7,198,951 for a discussion of various AAV serotypes). In certain embodiments the serotype is selected to optimize a desired mode of delivery.

Adeno-associated virus components suitable for inclusion in particles and vectors of the present invention include the capsid proteins, including the virion particle (VPs) proteins VP1, VP2, VP3, and hypervariable regions, the replication proteins (rep), including rep 78, rep 68, rep 52, and rep 40, and the sequences encoding these proteins. These components may be readily utilized in a variety of vector systems and cells. Such components may be used alone or in combination with other adeno-associated virus (AAV) serotype sequences or fragments, or in combination with elements from other AAV or non-AAV viral sequences. For example, in some embodiments a vector, nucleotide molecule, or particle of the present invention can comprise an AAV2 replication open reading frame (AAV2 Rep) in combination with an AAV7Retro, AAV9Retro, or AAV10Retro capsid open reading frame (AAV7Retro Cap, AAV9Retro Cap, and AAV10Retro Cap, respectively). Where, “Retro” indicates a capsid open reading frame (Cap) encoding a virion protein 1 (VP1) peptide comprising an amino acid sequence with about or at least about 80%, 85%, 90%, 95%, 96%, 97%, 98%, 99%, or 100% identity to LADQDYTKTA (SEQ ID NO: 11). In some embodiments, a retro-AAV2 (Tervo et al., Neuron 92: 372-382, 2016, which is incorporated herein by reference) comprises the amino acid sequence LADQDYTKTA (SEQ ID NO: 11).

In some embodiments, the recombinant adeno-associated virus (rAAV) particle comprises or is a vector. In some embodiments, the viral particle is a recombinant AAV particle comprising a nucleic acid comprising a heterologous gene flanked by one or two AAV inverted terminal repeats (ITRs). In various embodiments, the heterologous gene is encapsidated in the AAV particle. The AAV particle comprises capsid proteins. In some embodiments, the vector comprises a heterologous gene operatively linked to control sequences including promoters and transcription initiation and termination sequences, thereby forming an expression cassette.

### Polynucleotide Molecules

Recombinant adeno-associated virus (rAAV) vectors (alternatively, AAV vectors) of the invention comprise a nucleotide sequence (or polynucleotide) encoding an inhibitory nucleic acid molecule and any associated regulatory sequences (e.g., a promoter described herein and other control sequences described herein), and 5′ and 3′ adeno-associated virus (AAV inverted terminal repeats (ITRs). This recombinant adeno-associated virus vector can be packaged into a capsid protein encoded by a capsid open reading frame (Cap) and delivered to a selected target cell (e.g., a degenerating or damaged neuron). In some embodiments, the heterologous gene is a nucleic acid sequence, heterologous to the vector sequences, which encodes an inhibitory nucleic acid molecule (e.g., siRNA, miRNA, miRNA inhibitor) or other gene product, of interest. The nucleic acid coding sequence in certain embodiments is operatively linked to regulatory components in a manner which permits heterologous gene transcription, translation, and/or expression in a cell of a target tissue.

In some embodiments, the recombinant adeno-associated virus (AAV) vectors of the present invention comprise cis-acting 5′ and 3′ inverted terminal repeat (ITR) sequences described, e.g., by B. J. Carter, in “Handbook of Parvoviruses”, ed., P. Tijsser, CRC Press, pp. 155 168 (1990). The inverted terminal repeat (ITR) sequences can be about 50, 100, 125, 140, 145, or 150 bp in length. The ability to modify these inverted terminal repeat (ITR) sequences is within the skill of the art; see, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996). In various embodiments, a heterologous sequence comprised by a vector of the present invention and associated regulatory elements is flanked by 5′ and 3′ adeno-associated virus (AAV) inverted terminal repeat (ITR) sequences. The adeno-associated virus (AAV) inverted terminal repeat (ITR) sequences may be obtained from any known AAV, including, as non-limiting examples, AAV2, AAV7, AAV9, and AAV10.

In various embodiments, vectors of the present invention also include expression control sequences operably linked to the heterologous gene in a manner which permits transcription, translation and/or expression of the gene in a cell transfected with the vector(s) or infected with a virus particle of the invention. Thus, the present invention in various aspects provides an expression cassette. As used herein, “operably linked” sequences include both expression control sequences that are contiguous with the gene of interest (i.e., act in trans) and expression control sequences that act in trans or at a distance to control the gene of interest.

Expression control sequences include transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and sequences that enhance secretion of the encoded product. A great number of expression control sequences, including promoters which are native, constitutive, inducible and/or tissue-specific, are known in the art and are suitable for use in embodiments of the present invention. In some embodiments of the present invention a polyadenylation sequence can be inserted following a heterologous gene sequence. In various embodiments, the polyadenylation sequence is inserted before a 3′ adeno-associated virus (AAV) inverted terminal repeat (ITR) sequence. A rAAV vector useful in the present invention may also comprise an intron sequence. A non-limiting example of an intron sequence is an intron derived from SV-40, and is referred to as the SV-40 T intron sequence. Vectors of the present invention in various embodiments comprise an internal ribosome entry site (IRES). An IRES sequence is used to produce more than one polypeptide from a single gene transcript. An IRES sequence may be used to produce a protein that includes more than one polypeptide chain.

The precise nature of sequences needed for gene expression in host cells may vary between species, tissues or cell types. In some embodiments, vectors of the present invention comprise 5′ non-transcribed and 5′ non-translated sequences involved with the initiation of transcription and translation respectively of a heterologous gene, such as, to provide non-limiting examples, a TATA box, a capping sequence, a CAAT sequence, an enhancer elements, and the like. In various embodiments, a 5′ non-transcribed sequences can include a promoter region that includes a promoter sequence for transcriptional control of an operably joined gene. In some embodiments, vectors of the present invention include enhancer sequences or upstream activator sequences as desired. The vectors of the invention may optionally include 5′ leader or signal sequences. The choice and design of an appropriate vector is within the ability and discretion of one of ordinary skill in the art.

Examples of suitable promoters include, but are not limited to the retroviral Rous sarcoma virus (RSV) LTR promoter (optionally with the RSV enhancer), the cytomegalovirus (CMV) promoter (optionally with the CMV enhancer) (see, e.g., Boshart et al (1985) Cell, 41:521-530), the SV40 promoter, the dihydrofolate reductase promoter, the β-actin promoter (e.g., chicken β-actin promoter), the phosphoglycerol kinase (PGK) promoter, the EF1α promoter, the CBA promoter, UBC promoter, GUSB promoter, NSE promoter, Synapsin promoter, MeCP2 (methyl-CPG binding protein 2) promoter, GFAP; CBh promoter and the like. Exemplary promoters include, but are not limited to, the MoMLV LTR, a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken beta-actin/Rabbit β-globin promoter (CAG promoter; Niwa et al., Gene, 1991, 108(2):193-9) and the elongation factor 1-alpha promoter (EF1-alpha) promoter (Kim et al., Gene, 1990, 91(2):217-23 and Guo et al., Gene Ther., 1996, 3(9):802-10). In some embodiments, the promoter comprises a human β-glucuronidase promoter or a cytomegalovirus enhancer linked to a chicken β-actin (CBA) promoter. The promoter can be a constitutive, inducible, or repressible promoter. Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1u promoter [Invitrogen].

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter [Invitrogen]. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Non-limiting examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (see, e.g., WO 98/10088); the ecdysone insect promoter (see, e.g., No et al, Proc. Natl. Acad. Sci. USA, 93:3346-3351 (1996)), the tetracycline-repressible system (see, e.g., Gossen et al, Proc. Natl. Acad. Sci. USA, 89:5547-5551 (1992)), the tetracycline-inducible system (see, e.g., Gossen et al, Science, 268:1766-1769 (1995), and Harvey et al, Curr. Opin. Chem. Biol., 2:512-518 (1998)), the RU486-inducible system (see, e.g., Wang et al, Nat. Biotech., 15:239-243 (1997) and Wang et al, Gene Ther., 4:432-441 (1997)) and the rapamycin-inducible system (see, e.g., Magari et al, J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only. In another embodiment, the native promoter for a heterologous gene comprised by the vector will be used. The native promoter may be preferred when it is desired that expression of the heterologous gene should mimic the native expression. The native promoter may be used when expression of the heterologous gene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the promoter expresses the heterologous gene in a brain cell and/or in a cell body disposed in the brain. A brain cell may refer to any brain cell known in the art, including without limitation a neuron (such as a sensory neuron, motor neuron, interneuron, dopaminergic neuron, medium spiny neuron, cholinergic neuron, GABAergic neuron, pyramidal neuron, etc.), a glial cell (such as microglia, macroglia, astrocytes, oligodendrocytes, ependymal cells, radial glia, etc.), a brain parenchyma cell, microglial cell, ependymal cell, and/or a Purkinje cell. In some embodiments, the promoter expresses the heterologous gene in a neuron. In some embodiments, the heterologous gene is exclusively expressed in neurons (e.g., expressed in a neuron and not expressed in other cells of the CNS, such as glial cells).

In some embodiments, vectors of the present invention comprise expression control sequences imparting tissue-specific gene expression capabilities. In some cases, the tissue-specific expression control sequences bind tissue-specific transcription factors that induce transcription in a tissue specific manner. Exemplary tissue-specific regulatory sequences include, but are not limited to, the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter; alpha-fetoprotein (AFP) promoter, bone osteocalcin promoter; bone sialoprotein promoter, CD2 promoter; immunoglobulin heavy chain promoter; T cell receptor α-chain promoter, neuronal such as neuron-specific enolase (NSE) promoter, neurofilament light-chain gene promoter, and the neuron-specific vgf gene promoter. In some embodiments, the expression control sequence allows for specific expression in the central nervous system (CNS) or a subset of one or more neurons or other CNS cells.

In some embodiments, one or more inhibitory nucleic acids are incorporated in a gene of an adeno-associated virus vector, to inhibit the expression of a target gene in one or more tissues of a subject harboring the heterologous gene, e.g., non-central nervous system (CNS) tissues. In some embodiments, a vector of the present invention may comprise a replication open reading frame (Rep) from an adeno-associated virus (AAV) serotype that differs from that serotype which corresponds to a capsid open reading frame (Cap) comprised by the vector. In one embodiment, the Rep and Cap are expressed from separate sources (e.g., separate vectors, or a cell and a vector). In another embodiment, the Rep and Cap are fused in frame to one another to form a chimeric adeno-associated virus (AAV) vector, such as AAV2/7, AAV2/9, or AAV2/10. In some embodiments, an AAV1 Rep is fused in frame to Cap of the AAV serotype selected from the group consisting of AAV2, AAV3, AAV4, AAV5, AAV6, AAV6.2, AAV7, AAV7m8, AAV8, AAV9, AAV10, rh.10, rh.39, rh.43, and CSP3. In some embodiments, an AAV2 Rep is fused in frame to Cap of the AAV serotype selected from the group consisting of AAV1, AAV3, AAV4, AAV5, AAV6, AAV6.2, AAV7, AAV7m8, AAV8, AAV9, AAV10, rh.10, rh.39, rh.43, and CSP3. In some embodiments, an AAV3 Rep is fused in frame to Cap of the AAV serotype selected from the group consisting of AAV1, AAV2, AAV4, AAV5, AAV6, AAV6.2, AAV7, AAV7m8, AAV8, AAV9, AAV10, rh.10, rh.39, rh.43, and CSP3. In some embodiments, an AAV4 Rep is fused in frame to Cap of the AAV serotype selected from the group consisting of AAV1, AAV2, AAV3, AAV5, AAV6, AAV6.2, AAV7, AAV7m8, AAV8, AAV9, AAV10, rh.10, rh.39, rh.43, and CSP3. In some embodiments, an AAV5 Rep is fused in frame to Cap of the AAV serotype selected from the group consisting of AAV1, AAV2, AAV3, AAV4, AAV6, AAV6.2, AAV7, AAV7m8, AAV8, AAV9, AAV10, rh.10, rh.39, rh.43, and CSP3. In some embodiments, an AAV6 Rep protein is fused in frame to a Cap of the AAV serotype selected from the group consisting of AAV1, AAV2, AAV3, AAV4, AAV5, AAV6.2, AAV7, AAV7m8, AAV8, AAV9, AAV10, rh.10, rh.39, rh.43, and CSP3. In some embodiments, an AAV6.2 Rep protein is fused in frame to a Cap of the AAV serotype selected from the group consisting of AAV1, AAV2, AAV3, AAV4, AAV5, AAV6, AAV7, AAV7m8, AAV8, AAV9, AAV10, rh.10, rh.39, rh.43, and CSP3. In some embodiments, an AAV7 Rep protein is fused in frame to a Cap of the AAV serotype selected from the group consisting of AAV1, AAV2, AAV3, AAV4, AAV5, AAV6.2, AAV6.2, AAV8, AAV9, AAV10, rh.10, rh.39, rh.43, and CSP3. In some embodiments, an AAV7m8 Rep protein is fused in frame to a Cap of the AAV serotype selected from the group consisting of AAV1, AAV2, AAV3, AAV4, AAV5, AAV6.2, AAV6.2, AAV7, AAV8, AAV9, AAV10, rh.10, rh.39, rh.43, and CSP3. In some embodiments, an AAV8 Rep protein is fused in frame to a Cap of the AAV serotype selected from the group consisting of AAV1, AAV2, AAV3, AAV4, AAV5, AAV6.2, AAV6.2, AAV7, AAV7m8, AAV9, AAV10, rh.10, rh.39, rh.43, and CSP3. In some embodiments, an AAV9 Rep protein is fused in frame to a Cap of the AAV serotype selected from the group consisting of AAV1, AAV2, AAV3, AAV4, AAV5, AAV6.2, AAV6.2, AAV7, AAV7m8, AAV8, AAV10, rh.10, rh.39, rh.43, and CSP3. In some embodiments, an AAV10 Rep protein is fused in frame to a Cap of the AAV serotype selected from the group consisting of AAV1, AAV2, AAV3, AAV4, AAV5, AAV6.2, AAV6.2, AAV7, AAV7m8, AAV8, AAV9, rh.39, rh.43, and CSP3. In some embodiments, an AAVrh.39 Rep protein is fused in frame to a Cap of the AAV serotype selected from the group consisting of AAV1, AAV2, AAV3, AAV4, AAV5, AAV6.2, AAV6.2, AAV7, AAV7m8, AAV8, AAV9, AAV10, rh.10, rh.43, and CSP3. In some embodiments, an AAVrh.43 Rep protein is fused in frame to a Cap of the AAV serotype selected from the group consisting of AAV1, AAV2, AAV3, AAV4, AAV5, AAV6.2, AAV6.2, AAV7, AAV7m8, AAV8, AAV9, AAV10, rh.10, rh.39, and CSP3. In some embodiments, an AAVCSP3 Rep protein is fused in frame to a Cap of the AAV serotype selected from the group consisting of AAV1, AAV2, AAV3, AAV4, AAV5, AAV6.2, AAV6.2, AAV7, AAV7m7, AAV8, AAV9, AAV10, rh.10, rh.39, and rh.43.

### Polypeptide Expression

In order to express the inhibitory nucleic acids and gene editing systems described herein, DNA molecules obtained by any of the methods described herein or those that are known in the art, can be inserted into appropriate expression vectors by techniques well known in the art. For example, a double stranded DNA can be cloned into a suitable vector by restriction enzyme linking involving the use of synthetic DNA linkers or by blunt-ended ligation. DNA ligases are usually used to ligate the DNA molecules and undesirable joining can be avoided by treatment with alkaline phosphatase.

Therefore, the invention includes vectors (e.g., recombinant plasmids) that include nucleic acid molecules (e.g., genes or recombinant nucleic acid molecules encoding genes) as described herein. The term “recombinant vector” includes a vector (e.g., plasmid, phage, phasmid, virus, cosmid, fosmid, or other purified nucleic acid vector) that has been altered, modified or engineered such that it contains greater, fewer or different nucleic acid sequences than those included in the native or natural nucleic acid molecule from which the recombinant vector was derived. For example, a recombinant vector may include a gene, or fragment thereof, operatively linked to regulatory sequences, e.g., promoter sequences, terminator sequences, and the like, as defined herein. Recombinant vectors which allow for expression of the genes or nucleic acids included in them are referred to as “expression vectors.”

In some of the molecules of the invention described herein, one or more DNA molecules having a nucleotide sequence encoding one or more polypeptides of the invention are operatively linked to one or more regulatory sequences, which are capable of integrating the desired DNA molecule into a cell. Cells which have been stably transformed by the introduced DNA can be selected, for example, by introducing one or more markers which allow for selection of host cells which contain the expression vector. A selectable marker gene can either be linked directly to a nucleic acid sequence to be expressed, or be introduced into the same cell by co-transfection. Additional elements may also be needed for optimal synthesis of proteins described herein. It would be apparent to one of ordinary skill in the art which additional elements to use. It can be advantageous to codon-optimize a nucleotide sequence encoding one or more polypeptides of the invention for expression in a host organism. For example, a nucleotide sequence encoding a polypeptide of the invention can be codon-optimized for expression in a human cell. Also, polypeptide sequences of the invention can be humanized to facilitate expression in a human cell.

Factors of importance in selecting a particular plasmid or viral vector include, but are not limited to, the ease with which recipient cells that contain the vector are recognized and selected from those recipient cells which do not contain the vector; the number of copies of the vector which are desired in a particular host; and whether it is desirable to be able to “shuttle” the vector between host cells of different species.

Once the vector(s) is constructed to include a DNA sequence for expression, it may be introduced into an appropriate host cell by one or more of a variety of suitable methods that are known in the art, including but not limited to, for example, transformation, transfection, conjugation, protoplast fusion, electroporation, calcium phosphate-precipitation, direct microinjection, etc.

After the introduction of one or more vector(s), host cells are usually grown in a selective medium, which selects for the growth of vector-containing cells. Expression of recombinant proteins can be detected by immunoassays including Western blot analysis, immunoblot, and immunofluorescence. Purification of recombinant proteins can be carried out by any of the methods known in the art or described herein, for example, any conventional procedures involving extraction, precipitation, chromatography and electrophoresis. A further purification procedure that may be used for purifying proteins is affinity chromatography using monoclonal antibodies which bind a target protein. Generally, crude preparations containing a recombinant protein are passed through a column on which a suitable monoclonal antibody is immobilized. The protein usually binds to the column via the specific antibody while the impurities pass through. After washing the column, the protein is eluted from the gel by changing pH or ionic strength, for example.

### Genome Editing

Therapeutic gene editing is a major focus of biomedical research, embracing the interface between basic and clinical science. A degenerating or injured neuron may be treated according to the methods of the present invention by knocking out (e.g., by deletion) or inhibiting expression of a target gene(s). The development of novel “gene editing” tools provides the ability to manipulate the DNA sequence of a cell (e.g., to delete a target gene) at a specific chromosomal locus, without introducing mutations at other sites of the genome. This technology effectively enables the researcher to manipulate the genome of a subject's cells in vitro or in vivo.

In one embodiment, gene editing involves targeting an endonuclease (an enzyme that causes DNA breaks internally within a DNA molecule) to a specific site of the genome and thereby triggering formation of a chromosomal double strand break (DSB) at the chosen site. If, concomitant with the introduction of the chromosome breaks, a donor DNA molecule may be introduced (for example, by plasmid or oligonucleotide introduction), interactions between the broken chromosome and the introduced DNA can occur, especially if the two sequences share homology. In this instance, a process termed “gene targeting” can occur, in which the DNA ends of the chromosome invade homologous sequences of the donor DNA by homologous recombination (HR). By using the donor plasmid sequence as a template for HR, a seamless repair of the chromosomal DSB can be accomplished. In some embodiments, no donor DNA molecule is introduced and the double-stranded break is repaired by the error-prone non-homologous end joining NHEJ pathway leading to knock-out or deletion of the target gene (e.g., through the introduction of indels or nonsense mutations). In some embodiments, an endonuclease(s) can be targeted to at least two distinct chosen sites located within a gene sequence so that chromosomal double strand breaks at the distinct sites leads to excision and deletion of a nucleotide sequence flanked by the two distinct sites.

In some embodiments, the chosen site is associated with or disposed within a nucleotide sequence encoding a gene selected from one or more of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz. In some embodiments, more than one chosen site is selected. In some embodiments the chosen sites are associated with at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the foregoing genes. In some embodiments, the chosen site is associated with or disposed within a nucleotide sequence encoding a gene selected from one or more of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930. In some embodiments, the chosen site is associated with one or more of the 1, 2, 3, 4, 5, 6, 7, 8, 9, 10, 15, 20, 25, 30, 35, 40, 45, or 50 genes associated with the highest number of RBPMS+ cells/mm² in FIG. 6A. In some embodiments, the chosen sites selected are associated with or disposed within nucleotides sequences encoding a MAP2K7 gene and a MAP2K4 gene. In some embodiments, the chosen site is associated with or disposed within a nucleotide sequence encoding a Lkb1 gene.

In some embodiments, the chosen site is associated with or disposed within a nucleotide sequence encoding a gene selected from one or more of ATF3, ATF4, CEBPG, CHOP, Lkb1, Lpin2, Mkk4, Mkk7, Nt5c1a, SSRP1, and SUPT16. In some embodiments, more than one chosen site is selected. In some embodiments, the chosen sites are associated with at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the foregoing genes. In some embodiments, the chosen site is associated with or disposed within a nucleotide sequence encoding a gene selected from one or more of Fastkd5, Lhx6, Pawr, Rbbp7, Snrk, Stk10, Stradα, Tcf3, Tgif1, and Tie1. In some embodiments, the chosen site is associated with Snrk. In some embodiments, the chosen site is associated with one or more of the 1, 2, 3, 4, 5, 6, 7, 8, 9, 10, 15, 20, 25, or 30 genes associated with the highest normalized regenerating axon density (AU) in FIG. 6C.

In some embodiments where more than one chosen site is selected, a synergistic regenerative or cell (e.g., a neuron) survival effect can be achieved by selecting a chosen site associated with or disposed within a nucleotide sequence encoding a gene selected from one or more of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz and selecting at least one other distinct chosen site associated with or disposed within a nucleotide sequence encoding a gene selected from one or more of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930. In some embodiments, a synergistic effect can be effected by selecting one chosen site that is associated with an increase in cell survival (i.e., neuroprotection) and another chosen site that is associated with an increase in cell growth (i.e., neuroregeneration).

Current genome editing tools use the induction of double strand breaks (DSBs) to enhance gene manipulation of cells, including the deletion or knockout of genes. Such methods include zinc finger nucleases (ZFNs; described for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, and U.S. Pat. Publ. Nos. 20030232410 and US2009020314, which are incorporated herein by reference), Transcription Activator-Like Effector Nucleases (TALENs; described for example in U.S. Pat. Nos. 8,440,431, 8,440,432, 8,450,471, 8,586,363, and 8,697,853, and U.S. Pat. Publ. Nos. 20110145940, 20120178131, 20120178169, 20120214228, 20130122581, 20140335592, and 20140335618, which are incorporated herein by reference), and the CRISPR (Clustered Regularly Interspaced Short Palindromic Repeats)/Cas9 system (described for example in U.S. Pat. Nos. 8,697,359, 8,771,945, 8,795,965, 8,871,445, 8,889,356, 8,906,616, 8,932,814, 8,945,839, 8,993,233, and 8,999,641, and U.S. Pat. Publ. Nos. 20140170753, 20140227787, 20140179006, 20140189896, 20140273231, 20140242664, 20140273232, 20150184139, 20150203872, 20150031134, 20150079681, 20150232882, and 20150247150, which are incorporated herein by reference). In some embodiments a CRISPR/Cas12 system can be used for gene editing. In some embodiments, the Cas12 polypeptide is Cas12b. In some embodiments any Cas polypeptide can be used for gene editing (e.g., CasX). In various embodiments, the Cas polypeptide is selected so that a nucleotide encoding the Cas poypeptide can fit within an adeno-associated virus (AAV) capsid. For example, ZFN DNA sequence recognition capabilities and specificity can be unpredictable. Similarly, TALENs and CRISPR/Cas9 cleave not only at the desired site, but often at other “off-target” sites, as well. These methods have significant issues connected with off-target double-stranded break induction and the potential for deleterious mutations, including indels, genomic rearrangements, and chromosomal rearrangements, associated with these off-target effects. ZFNs and TALENs entail use of modular sequence-specific DNA binding proteins to generate specificity for −18 bp sequences in the genome. CRISPR/Cas9, TALENs, and ZFNs have all been used in clinical trials (see, e.g., Li., H, et al., “Applications of genome editing technology in the targeted therapy of human diseases: mechanisms, advances and prospects”, Signal Transduct Target Ther., 5:1 (2020), DOI: 10.1038/s41392-019-0089-y).

RNA-guided nucleases-mediated genome editing, based on Type 2 CRISPR (Clustered Regularly Interspaced Short Palindromic Repeat)/Cas (CRISPR Associated) systems, offers a valuable approach to alter the genome. In brief, Cas9, a nuclease guided by single-guide RNA (sgRNA), binds to a targeted genomic locus next to the protospacer adjacent motif (PAM) and generates a double-strand break (DSB). The DSB is then repaired either by non-homologous end joining (NHEJ), which leads to insertion/deletion (indel) mutations, or by homology-directed repair (HDR), which requires an exogenous template and can generate a precise modification at a target locus (Mali et al., Science. 2013 Feb. 15; 339(6121):823-6). Genetic manipulation using engineered nucleases has been demonstrated in tissue culture cells and rodent models of diseases.

CRISPR has been used in a wide range of organisms including baker's yeast (S. cerevisiae), zebra fish, nematodes (C. elegans), plants, mice, and several other organisms. Additionally, CRISPR has been modified to make programmable transcription factors that allow scientists to target and activate or silence specific genes. Libraries of tens of thousands of guide RNAs are now available.

Since 2012, the CRISPR/Cas system has been used for gene editing (silencing, enhancing or changing specific genes) that even works in eukaryotes like mice and primates. By inserting a plasmid containing Cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of Cas genes and repeat structures have been used to define 8 CRISPR subtypes (E. coli, Y. pest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (about 30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E. coli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes. See also U.S. Patent Publication 2014/0068797, which is incorporated by reference in its entirety.

Cas9

Cas9 is a nuclease, an enzyme specialized for cutting DNA, with two active cutting sites, one for each strand of the double helix. The team demonstrated that they could disable one or both sites while preserving Cas9's ability to home located its target DNA. Jinek et al. (2012) combined tracrRNA and spacer RNA into a “single-guide RNA” molecule that, mixed with Cas9, could find and cut the correct DNA targets. It has been proposed that such synthetic guide RNAs might be able to be used for gene editing (Jinek et al., Science. 2012 Aug. 17; 337(6096):816-21).

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Coinjection of Cas9 mRNA and sgRNAs into the germline (zygotes) generated mice with mutations. Delivery of Cas9 DNA sequences also is contemplated.

Cas9 variants have been developed or discovered that can fit into an adeno-associated virus (AAV) capsid with sgRNA. Non-limiting examples of such variants (e.g., Cas9 orthologs) suitable for use in embodiments of the invention of the disclosure include saCas9 (Staphylococcus aureus Cas9), cjCas9 (Camphylobacter jejuni Cas9), NmeCas9 (Neisseria meningitidis Cas9), and spCas9 (Streptococcus pyrogenes Cas 9). An example of a saCas9 suitable for delivery by an AAV vector is provided in Ann Ran, F. et al. “In vivo genome editing using Staphylococcus aureus Cas9”, Nature, 9:186-91, DOI: 10.1038/nature14299.

gRNA

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type 21 promoter U6). Synthetic gRNAs are slightly over 100 bp at the minimum length and contain a portion which is targets the 20 protospacer nucleotides immediately preceding the PAM sequence NGG; gRNAs do not contain a PAM sequence.

CRISPR Interference

In some embodiments, a target gene can be inhibited using CRISPR interference (CRISPRi). CRISPRi is a technique where expression of a target gene is inhibited by the binding of a nuclease-inactive CRISPR system (a CRISPRi system), optionally comprising transcriptional repressors. In some embodiments, the method of CRISPRi involves designing an sgRNA complementary to a promoter or exonic sequence of a target gene. In some embodiments, CRISPRi involves guiding a transcriptional repressor to a transcription start site of a target gene. CRISPRi has been successfully used for the repression of gene expression in mice and an exemplary method for using CRISPRi to repress a gene is provided in MacLeod, et al., “Effective CRISPR interference of an endogenous gene via a single transgene in mice”, Scientific Reports, 9:17312 (2019).

### Small Molecule Inhibitors

The inhibition of any one of the following genes resulted in increased survival, a reduction in cell death or protection from neuron degeneration: ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz. In embodiments, inhibition of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or all of the foregoing genes resulted in increased neuron survival, reduction in neuron death, or protection from neuron degeneration.

The inhibition of any one of the following genes results in an increase in axon regrowth or neuroregeneration: Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930. In embodiments, inhibition of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the foregoing genes resulted in neuroregeneration (e.g., axon regrowth). Thus, provided herein are inhibitory agents that inhibit expression of such genes or inhibit an activity of molecular target associated with (e.g., a polypeptide or polynucleotide encoded by) such genes. In some embodiments, the agent comprises a small molecule compound (e.g., those listed below). Such agents can be delivered to cells of a subject having a neurodegenerative disease or a degenerating/degenerating neuron. The compounds are delivered to the cells of a subject in a form in which they can be taken up so that therapeutically effective levels of the agent is introduced to a cell.

In some embodiments, the inhibitory agent comprises one or more of the agents listed in Table 1 or discussed elsewhere herein. In some embodiments, the agent is a direct inhibitor of the target gene or molecule. In some embodiments, the agent is an indirect inhibitor of the target gene or molecule.

TABLE 1
Small molecule compounds suitable for use in pharmaceutical
compositions of the present disclosure.

| Molecular/ | | |
|---|---|---|
| Gene target | Name of small-molecule | Source/Reference |
| Carf | Soyasapogenol-A | Scientific Reports 10: 6323 (2020), |
| | | DOI: 10.1038/s41598-020-62953-5 |
| Cdk9 | A-1467729 | Leukemia, 34: 1646-1657 (2020), |
| | | DOI: 10.1038/s41375-019-0652-0 |
| Cdk9 | A-1592668 | Leukemia, 34: 1646-1657 (2020), |
| | | DOI: 10.1038/s41375-019-0652-0 |
| Cebp | Withaferin A | Biochimica et Biophysica Acta, |
| | | 1864: 1349-1358 (2017), DOI: |
| | | 10.1016/j.bbamcr.2017.05.003 |
| ep300 | Y08197 | Acta Pharmacologica Sinica 40: 1436- |
| | | 1447 (2019), DOI: 10.1038/s41401- |
| | | 019-0237-5 |
| Fbp1 | 2,5-dichloro-N-(5-chloro-2- | Cayman Chemical Co. |
| | benzoxazolyl)-benzenesulfonamide | |
| | (CAS: 883973-99-7) | |
| Fgfr | Sorafenib Tosylate | Spelleck Chemicals LLC |
| Fgfr | Ponatinib (AP24534) | Spelleck Chemicals LLC |
| Fgfr | Infigratinib (BGJ398) | Spelleck Chemicals LLC |
| Fgfr | Nintedanib (BIBF 1120) | Spelleck Chemicals LLC |
| Fgfr | Pazopanib HCl (GW786034 HCl) | Spelleck Chemicals LLC |
| Fgfr | PD173074 | Spelleck Chemicals LLC |
| Fgfr | Dovitinib (TKI-258) | Spelleck Chemicals LLC |
| Fgfr | AZD4547 | Spelleck Chemicals LLC |
| Fgfr | Danusertib (PHA-739358) | Spelleck Chemicals LLC |
| Fgfr | Lenvatinib (E7080) | Spelleck Chemicals LLC |
| Fgfr | Brivanib (BMS-540215) | Spelleck Chemicals LLC |
| Fgfr | ENMD-2076 | Spelleck Chemicals LLC |
| Fgfr | Pazopanib | Spelleck Chemicals LLC |
| Fgfr | MK-2461 | Spelleck Chemicals LLC |
| Fgfr | Brivanib Alaninate (BMS-582664) | Spelleck Chemicals LLC |
| Fgfr | Tyrphostin AG 1296 | Spelleck Chemicals LLC |
| Fgfr | SSR128129E | Spelleck Chemicals LLC |
| Fgfr | Sulfatinib | Spelleck Chemicals LLC |
| Fgfr | Pemigatinib (INCB054828) | Spelleck Chemicals LLC |
| Fgfr | ODM-203 | Spelleck Chemicals LLC |
| Fgfr | Futibatinib (TAS-120) | Spelleck Chemicals LLC |
| Fgfr | ASP5878 | Spelleck Chemicals LLC |
| Fgfr | Derazantinib(ARQ-087) | Spelleck Chemicals LLC |
| Fgfr | lenvatinib Mesylate | Spelleck Chemicals LLC |
| Fgfr | Nintedanib Ethanesulfonate Salt | Spelleck Chemicals LLC |
| Fgfr | H3B-6527 | Spelleck Chemicals LLC |
| Fgfr | Roblitinib (FGF401) | Spelleck Chemicals LLC |
| Fgfr | PRN1371 | Spelleck Chemicals LLC |
| Fgfr | PD-166866 (PD166866) | Spelleck Chemicals LLC |
| Fgfr | Fisogatinib (BLU-554) | Spelleck Chemicals LLC |
| Fgfr | S49076 | Spelleck Chemicals LLC |
| Fgfr | NSC12 | Spelleck Chemicals LLC |
| Fgfr | ON123300 | Spelleck Chemicals LLC |
| Fgfr | SU5402 | Spelleck Chemicals LLC |
| Fgfr | BLU9931 | Spelleck Chemicals LLC |
| Fgfr | FIIN-2 | Spelleck Chemicals LLC |
| Fgfr | Dovitinib (TKI258) Lactate | Spelleck Chemicals LLC |
| Fgfr | CH5183284 (Debio-1347) | Spelleck Chemicals LLC |
| Fgfr | LY2874455 | Spelleck Chemicals LLC |
| Fgfr | Ferulic Acid | Spelleck Chemicals LLC |
| Fgfr | BO-264 | Spelleck Chemicals LLC |
| Fgfr | SKLB 610 | Spelleck Chemicals LLC |
| Fgfr | Alofanib(RPT835) | Spelleck Chemicals LLC |
| Fgfr | Gambogenic acid | Spelleck Chemicals LLC |
| Fgfr | Erdafitinib (JNJ-42756493) | Spelleck Chemicals LLC |
| Flt1 | Sunitinib Malate (CAS: 341031-54-7) | Santa Cruz Biotechnology, Inc. |
| Flt1 | AAL-993 (CAS: 269390-77-4) | Santa Cruz Biotechnology, Inc. |
| Flt1 | PKC-412 (CAS: 120685-11-2) | Santa Cruz Biotechnology, Inc. |
| Flt1 | DMXAA (CAS: 117570-53-3) | Santa Cruz Biotechnology, Inc. |
| Flt1 | VEGFR Tyrosine Kinase Inhibitor III, | Santa Cruz Biotechnology, Inc. |
| | KRN633 (CAS: 286370-15-8) | |
| Flt1 | Foretinib (CAS: 849217-64-7) | Santa Cruz Biotechnology, Inc. |
| Flt1 | AZ628 (CAS: 878739-06-1) | Santa Cruz Biotechnology, Inc. |
| Flt1 | BIBF1120 (CAS: 656247-17-5) | Santa Cruz Biotechnology, Inc. |
| Flt1 | Motesanib Diphosphate (CAS: | Santa Cruz Biotechnology, Inc. |
| | 857876-30-3) | |
| Flt1 | SU 4312 (CAS: 5812-07-7) | Santa Cruz Biotechnology, Inc. |
| Flt1 | VEGFR Tyrosine Kinase Inhibitor II | Santa Cruz Biotechnology, Inc. |
| | (CAS: 269390-69-4) | |
| Flt1 | ZM-306416 (CAS: 690206-97-4) | Santa Cruz Biotechnology, Inc. |
| Flt1 | ABT-869 (CAS: 796967-16-3) | Santa Cruz Biotechnology, Inc. |
| Flt1 | 4,4prime-Bis(4- | Santa Cruz Biotechnology, Inc. |
| | aminophenoxy)biphenyl (CAS: | |
| | 13080-85-8) | |
| Flt1 | MGCD-265 (CAS: 875337-44-3) | Santa Cruz Biotechnology, Inc. |
| Flt1 | Sunitinib, Free Base (CAS: 557795- | Santa Cruz Biotechnology, Inc. |
| | 19-4) | |
| Flt1 | Pazopanib Hydrochloride (CAS: | Santa Cruz Biotechnology, Inc. |
| | 635702-64-6) | |
| Flt1 | Brivanib (CAS: 649735-46-6) | Santa Cruz Biotechnology, Inc. |
| Frk | SU 4984 | EP1372621 |
| Lkb1/Strada | temsirolimus | Curr Drug Targets, 15: 32-52 (2014) |
| Lkb1/Strada | everolimus (rapamycin) | Curr Drug Targets, 15: 32-52 (2014) |
| Lkb1/Strada | ridaforolimus | Curr Drug Targets, 15: 32-52 (2014) |
| Lkb1/Strada | 2-DG metformin phenformin | Curr Drug Targets, 15: 32-52 (2014) |
| Lkb1/Strada | 17-AAG | Curr Drug Targets, 15: 32-52 (2014) |
| Lkb1/Strada | geldanamycin | Curr Drug Targets, 15: 32-52 (2014) |
| Lkb1/Strada | novobicin | Curr Drug Targets, 15: 32-52 (2014) |
| Lkb1/Strada | XL888 | Curr Drug Targets, 15: 32-52 (2014) |
| Lkb1/Strada | celecoxib | Curr Drug Targets, 15: 32-52 (2014) |
| Lkb1/Strada | non-steroidal anti-inflamatory drugs | Curr Drug Targets, 15: 32-52 (2014) |
| Lkb1/Strada | dasatinib | Curr Drug Targets, 15: 32-52 (2014) |
| Map2k4/MKK4 | BSJ-04-122 | Cell Chem. Biol. 27: P1553-1560.E8, |
| | | DOI: |
| | | 10.1016/j.chembiol.2020.08.014 |
| Map2k7/MKK7 | MKK7-COV-1 | Cell Chem. Biol. 26: P98-108.E5 |
| | | (2019), DOI: |
| | | 10.1016/j.chembiol.2018.10.011 |
| Map2k7/MKK7 | MKK7-DOCK-1 | Cell Chem. Biol. 26: P98-108.E5 |
| | | (2019), DOI: |
| | | 10.1016/j.chembiol.2018.10.011 |
| Map2k7/MKK7 | MKKY-DOCK-2 | Cell Chem. Biol. 26: P98-108.E5 |
| | | (2019), DOI: |
| | | 10.1016/j.chembiol.2018.10.011 |
| Map2k7/MKK7 | MKKY-COV-23 | Cell Chem. Biol. 26: P98-108.E5 |
| | | (2019), DOI: |
| | | 10.1016/j.chembiol.2018.10.011 |
| Map2k7/MKK7 | MKK7-COV-2 | Cell Chem. Biol. 26: P98-108.E5 |
| | | (2019), DOI: |
| | | 10.1016/j.chembiol.2018.10.011 |
| Map2k7/MKK7 | MKK7-COV-4 | Cell Chem. Biol. 26: P98-108.E5 |
| | | (2019), DOI: |
| | | 10.1016/j.chembiol.2018.10.011 |
| Map2k7/MKK7 | MKK7-COV-14 | Cell Chem. Biol. 26: P98-108.E5 |
| | | (2019), DOI: |
| | | 10.1016/j.chembiol.2018.10.011 |
| Map2k7/MKK7 | MKK7-COV-5 | Cell Chem. Biol. 26: P98-108.E5 |
| | | (2019), DOI: |
| | | 10.1016/j.chembiol.2018.10.011 |
| Map2k7/MKK7 | MKK7-COV-15 | Cell Chem. Biol. 26: P98-108.E5 |
| | | (2019), DOI: |
| | | 10.1016/j.chembiol.2018.10.011 |
| Map2k7/MKK7 | MKK7-COV-16 | Cell Chem. Biol. 26: P98-108.E5 |
| | | (2019), DOI: |
| | | 10.1016/j.chembiol.2018.10.011 |
| Map2k7/MKK7 | MKK7-COV-17 | Cell Chem. Biol. 26: P98-108.E5 |
| | | (2019), DOI: |
| | | 10.1016/j.chembiol.2018.10.011 |
| Map2k7/MKK7 | MKK7-COV-18 | Cell Chem. Biol. 26: P98-108.E5 |
| | | (2019), DOI: |
| | | 10.1016/j.chembiol.2018.10.011 |
| Map2k7/MKK7 | MKK7-COV-19 | Cell Chem. Biol. 26: P98-108.E5 |
| | | (2019), DOI: |
| | | 10.1016/j.chembiol.2018.10.011 |
| Map2k7/MKK7 | MKK7-COV-27 | Cell Chem. Biol. 26: P98-108.E5 |
| | | (2019), DOI: |
| | | 10.1016/j.chembiol.2018.10.011 |
| Map2k7/MKK7 | MKK7-COV-3 | Cell Chem. Biol. 26: P98-108.E5 |
| | | (2019), DOI: |
| | | 10.1016/j.chembiol.2018.10.011 |
| Map2k7/MKK7 | MKK7-COV-21 | Cell Chem. Biol. 26: P98-108.E5 |
| | | (2019), DOI: |
| | | 10.1016/j.chembiol.2018.10.011 |
| Map2k7/MKK7 | MKK7-COV-20 | Cell Chem. Biol. 26: P98-108.E5 |
| | | (2019), DOI: |
| | | 10.1016/j.chembiol.2018.10.011 |
| Map2k7/MKK7 | MKK7-COV-22 | Cell Chem. Biol. 26: P98-108.E5 |
| | | (2019), DOI: |
| | | 10.1016/j.chembiol.2018.10.011 |
| Map2k7/MKK7 | MKK7-COV-13 | Cell Chem. Biol. 26: P98-108.E5 |
| | | (2019), DOI: |
| | | 10.1016/j.chembiol.2018.10.011 |
| Map2k7/MKK7 | MKK7-COV-25 | Cell Chem. Biol. 26: P98-108.E5 |
| | | (2019), DOI: |
| | | 10.1016/j.chembiol.2018.10.011 |
| Map2k7/MKK7 | MKK7-COV-24 | Cell Chem. Biol. 26: P98-108.E5 |
| | | (2019), DOI: |
| | | 10.1016/j.chembiol.2018.10.011 |
| Map2k7/MKK7 | MKK7-COV-26 | Cell Chem. Biol. 26: P98-108.E5 |
| | | (2019), DOI: |
| | | 10.1016/j.chembiol.2018.10.011 |
| Map2k7/MKK7 | MKK7-COV-1 | Cell Chem. Biol. 26: P98-108.E5 |
| | | (2019), DOI: |
| | | 10.1016/j.chembiol.2018.10.011 |
| Map2k7/MKK7 | MKK7-COV-2 | Cell Chem. Biol. 26: P98-108.E5 |
| | | (2019), DOI: |
| | | 10.1016/j.chembiol.2018.10.011 |
| Map2k7/MKK7 | MKK7-COV-3 | Cell Chem. Biol. 26: P98-108.E5 |
| | | (2019), DOI: |
| | | 10.1016/j.chembiol.2018.10.011 |
| Map2k7/MKK7 | MKK7-COV-7 | Cell Chem. Biol. 26: P98-108.E5 |
| | | (2019), DOI: |
| | | 10.1016/j.chembiol.2018.10.011 |
| Map2k7/MKK7 | MKK7-COV-8 | Cell Chem. Biol. 26: P98-108.E5 |
| | | (2019), DOI: |
| | | 10.1016/j.chembiol.2018.10.011 |
| Map2k7/MKK7 | MKK7-COV-9 | Cell Chem. Biol. 26: P98-108.E5 |
| | | (2019), DOI: |
| | | 10.1016/j.chembiol.2018.10.011 |
| Map2k7/MKK7 | MKK7-COV-28 | Cell Chem. Biol. 26: P98-108.E5 |
| | | (2019), DOI: |
| | | 10.1016/j.chembiol.2018.10.011 |
| Map2k7/MKK7 | MKK7-COV-12 | Cell Chem. Biol. 26: P98-108.E5 |
| | | (2019), DOI: |
| | | 10.1016/j.chembiol.2018.10.011 |
| Map2k7/MKK7 | MKK7-COV-29 | Cell Chem. Biol. 26: P98-108.E5 |
| | | (2019), DOI: |
| | | 10.1016/j.chembiol.2018.10.011 |
| Map2k7/MKK7 | MKK7-NEG-1 | Cell Chem. Biol. 26: P98-108.E5 |
| | | (2019), DOI: |
| | | 10.1016/j.chembiol.2018.10.011 |
| Map2k7/MKK7 | MKK7-NEG-2 | Cell Chem. Biol. 26: P98-108.E5 |
| | | (2019), DOI: |
| | | 10.1016/j.chembiol.2018.10.011 |
| Pld2 | CAY10594 | Scientific Reports 9: 7242 (2019), |
| | | DOI: 10.1038/s41598-019-43673-x |
| Ptpn2 | PTP inhibitor XIX | BMB Rep. 40: 329-334 (2017), DOI: |
| | | 10.5483/bmbrep.2017.50.6.003 |
| Ripk | Name | AbMole Bioscience |
| Ripk | RIPA-56 | AbMole Bioscience |
| Ripk | GSK2982772 | AbMole Bioscience |
| Ripk | GSK2983559 free acid | AbMole Bioscience |
| Ripk | HS-1371 | AbMole Bioscience |
| Ripk | GSK872 | AbMole Bioscience |
| Ripk | WEHI-345 | AbMole Bioscience |
| Ripk | Necrostatin-1 | AbMole Bioscience |
| Rngtt | Mizoribine | WO2016141297A2 |
| Sin3 | selamectin | Mol Cancer Ther., 14: 1824-1836 |
| | | (2015), DOI: 10.1158/1535- |
| | | 7163.MCT-14-0980-T |
| Sin3 | ivermectin | Mol Cancer Ther., 14: 1824-1836 |
| | | (2015), DOI: 10.1158/1535- |
| | | 7163.MCT-14-0980-T |
| Sin3 | an avermectin | Mol Cancer Ther., 14: 1824-1836 |
| | | (2015), DOI: 10.1158/1535- |
| | | 7163.MCT-14-0980-T |
| Sin3 | an avermectin marocyclic lactone | Mol Cancer Ther., 14: 1824-1836 |
| | derivative | (2015), DOI: 10.1158/1535- |
| | | 7163.MCT-14-0980-T |
| Sin3 | a macrocyclic lactone | Mol Cancer Ther., 14: 1824-1836 |
| | | (2015), DOI: 10.1158/1535- |
| | | 7163.MCT-14-0980-T |
| Snrk | staurosporine | IUPHAR/BPS Guide to Pharmacology |
| Snrk | fedratinib | IUPHAR/BPS Guide to Pharmacology |
| Snrk | NVP-TAE684 | IUPHAR/BPS Guide to Pharmacology |
| Snrk | sunitinib | IUPHAR/BPS Guide to Pharmacology |
| Snrk | SU-14813 | IUPHAR/BPS Guide to Pharmacology |
| Snrk | SB203580 | IUPHAR/BPS Guide to Pharmacology |
| Snrk | ruboxistaurin | IUPHAR/BPS Guide to Pharmacology |
| Snrk | erlotinib | IUPHAR/BPS Guide to Pharmacology |
| Snrk | GSK690693 | IUPHAR/BPS Guide to Pharmacology |
| Snrk | masitinib | IUPHAR/BPS Guide to Pharmacology |
| Srpk2 | SRPKIN-1 | Cell Chem Biol. 25: 460-470 (2018), |
| | | DOI: |
| | | 10.1016/j.chembiol.2018.01.013 |
| STK10 | SB-633825 | MedChemExpress LLC |
| Stk381 | the calcium ionophore A23187 | Scientific Reports, 9: 16010 (2019), |
| | | DOI: 10.1038/s41598-019-52435-8 |
| SUPT16 | CBL0100 | Sci Transl Med 3: 95ra74 (2011), DOI: |
| | | 10.1126/scitranslmed.3002530; |
| | | Stem Cells Dev., 15: 1693-1701, DOI: |
| | | 10.1089/scd.2018.0150 |
| SUPT16 | a FACT inhibitor | Sci Transl Med 3: 95ra74 (2011), DOI: |
| | | 10.1126/scitranslmed.3002530; |
| | | Stem Cells Dev., 15: 1693-1701, DOI: |
| | | 10.1089/scd.2018.0150 |

In some embodiments the inhibitory agent is a small molecule inhibitor of Map2k7/MKK7 and has one of the following chemical structures:

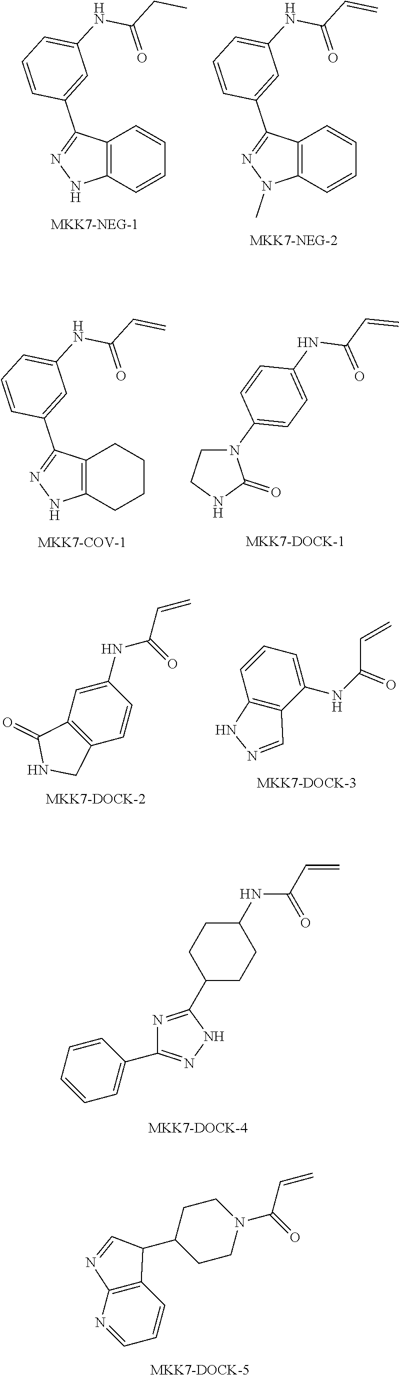

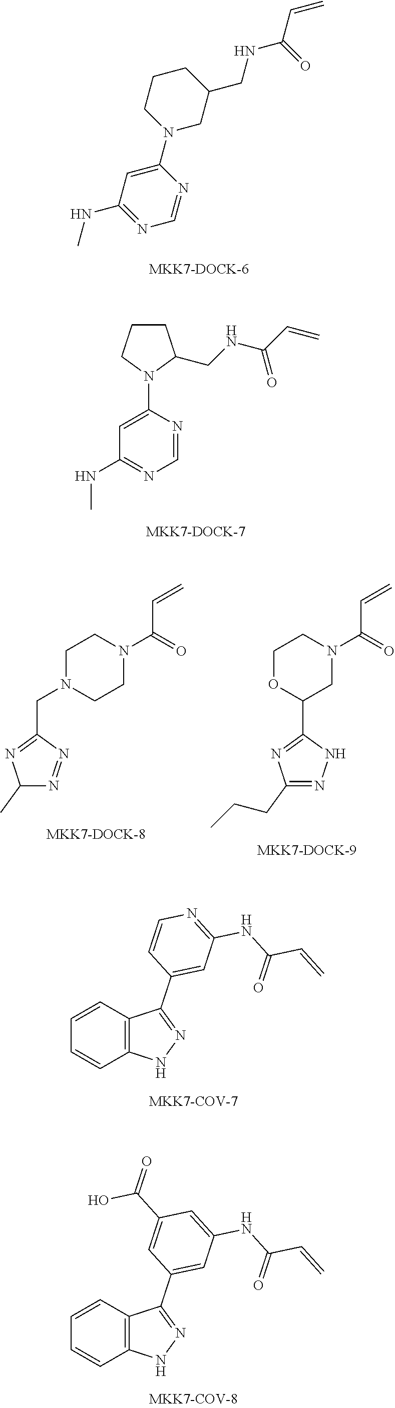

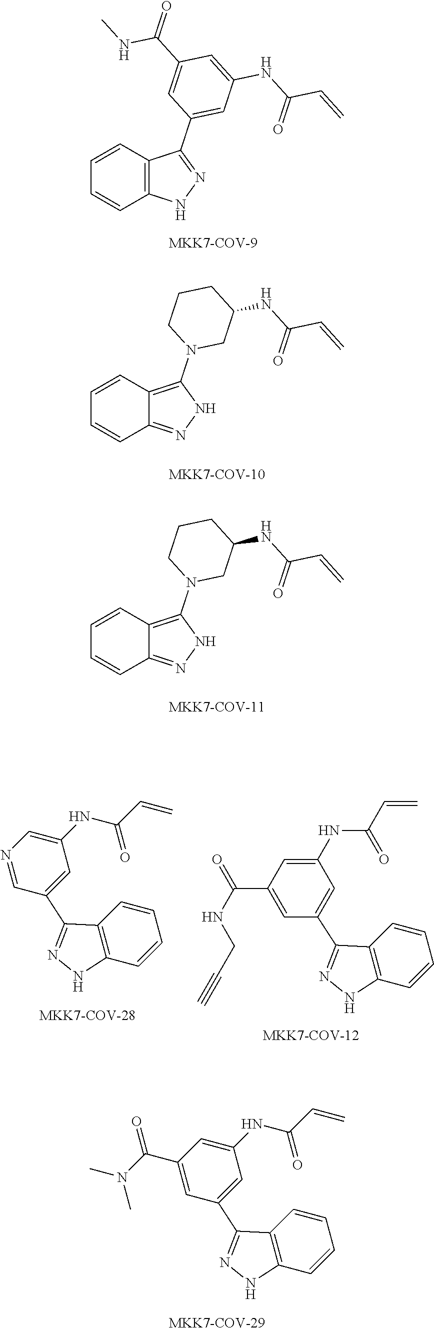 [SMILES: C=CC(=O)Nc1cc(C(=O)NC)cc(-c2n[nH]c3ccccc23)c1 ; C=CC(=O)N[C@H]1CCCN(c2[nH]nc3ccccc23)C1 ; C=CC(=O)N[C@@H]1CCCN(c2[nH]nc3ccccc23)C1 ; C=CC(=O)Nc1cncc(-c2n[nH]c3ccccc23)c1 ; C#CCNC(=O)c1cc(NC(=O)C=C)cc(-c2n[nH]c3ccccc23)c1 ; C=CC(=O)Nc1cc(C(=O)N(C)C)cc(-c2n[nH]c3ccccc23)c1]

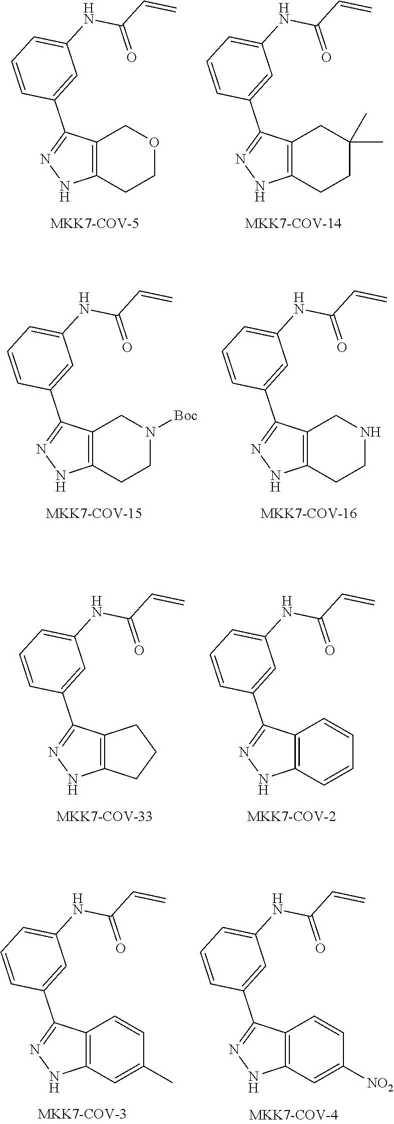

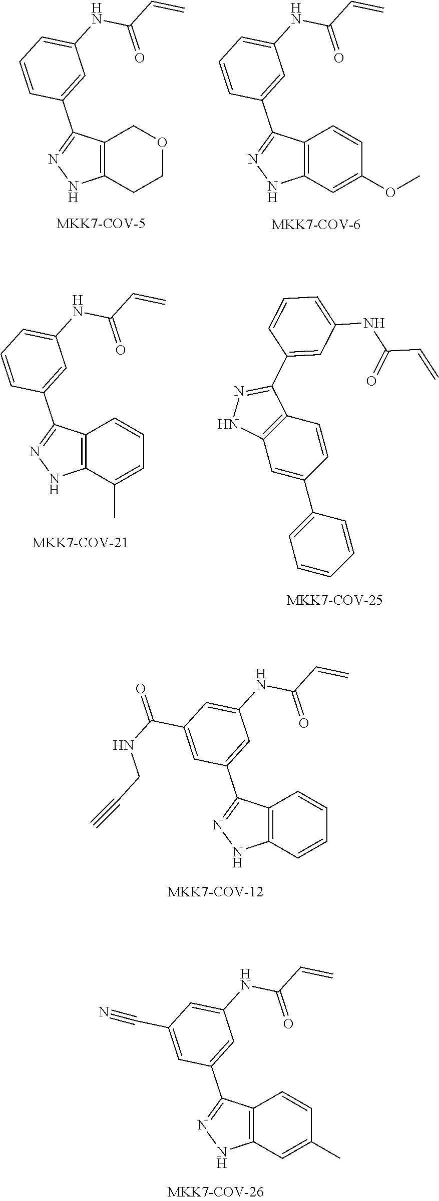 [SMILES: C=CC(=O)Nc1cccc(-c2n[nH]c3c2COCC3)c1 ; C=CC(=O)Nc1cccc(-c2n[nH]c3cc(OC)ccc23)c1 ; C=CC(=O)Nc1cccc(-c2n[nH]c3c(C)cccc23)c1 ; C=CC(=O)Nc1cccc(-c2n[nH]c3cc(-c4ccccc4)ccc23)c1 ; C#CCNC(=O)c1cc(NC(=O)C=C)cc(-c2n[nH]c3ccccc23)c1 ; C=CC(=O)Nc1cc(C#N)cc(-c2n[nH]c3cc(C)ccc23)c1]

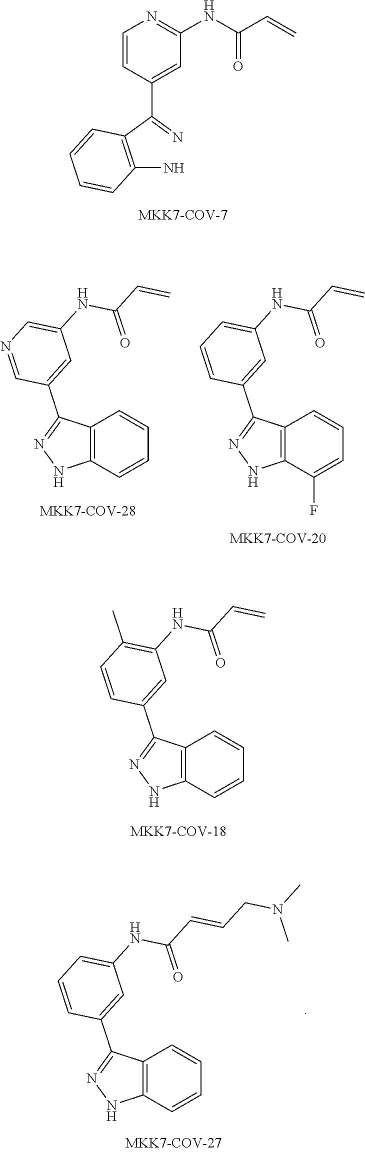 [SMILES: C=CC(=O)Nc1cc(C(=N)c2ccccc2N)ccn1 ; C=CC(=O)Nc1cncc(-c2n[nH]c3ccccc23)c1 ; C=CC(=O)Nc1cccc(-c2n[nH]c3c(F)cccc23)c1 ; C=CC(=O)Nc1cc(-c2n[nH]c3ccccc23)ccc1C ; CN(C)C/C=C/C(=O)Nc1cccc(-c2n[nH]c3ccccc23)c1]

In some embodiments, the inhibitory agent is a small molecule inhibitor of Map2k7/MKK7 and has the structure

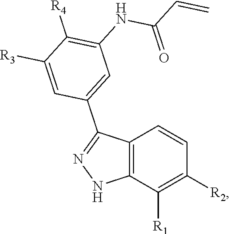

where R₁ is independently H, F, CH₃, Cl, or NO₂, R₂ is independently H, CH₃, NO₂, OMe, Br, Cl, or PH, R₃ is independently H or CN, and R₄ is independently H or CH₃.

BSJ-04-122 has the structure

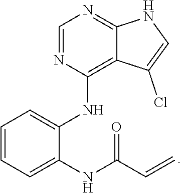 [SMILES: C=CC(=O)Nc1ccccc1Nc1ncnc2[nH]cc(Cl)c12]

In some embodiments, BSJ-04-122 is a small molecule inhibitor of the target gene MKK4 or a molecular target associated therewith.

In some embodiments, a small molecule inhibitor of the target gene Chop or a small molecule inhibitor associated therewith is one of the small molecule inhibitors disclosed by PubChem [Internet]. Bethesda (MD): National Library of Medicine (US), National Center for Biotechnology Information; 2004-. PubChem Bioassay Record for AID 2732, Source: Emory University Molecular Libraries Screening Center.

### Transplantation of Neurons

Transplantation of neurons into a subject can lead to death of the transplanted neurons, while the growth of the transplanted cells into the host tissue is critical for establishment of functional connections between the grafted cells and the host neurons. Therefore, the present invention provides neurons that have been treated by any of the methods provided herein and/or with any of the agents described herein to reduce or eliminate expression of a target gene and/or to inhibit or eliminate activity of a molecular target. Such neurons in various embodiments have an increased capacity for survival and/or growth following transplantation relative to a reference neuron(s) that have not been treated according to the methods described herein. In some embodiments, the neuron is a stem cell or donor cell-derived neuron. Representative methods for preparing a human neuron cell from a donor cell are provided in Andrew S. Yoo et al., “MicroRNAs Induce a Permissive Chromatin Environment that Enables Neuronal Subtype-Specific Reprogramming of Adult Human Fibroblasts.” Cell Stem Cell, September 2017, DOI: 10.1016/j.stem.2017.08.002, the disclosure of which is incorporated herein in its entirety for all purposes. Examples and methods of neuron cell transplantation and cells suitable for use in such transplantation into a subject are provided in Li., J. and Lepski, G., “Cell Transplantation for Spinal Cord Injury: A Systematic Review”, BioMed Research International, vol. 2013, Article ID 786475, DOI: 10.1155/2013/786475; in Borlongan, C., “Concise Review: Stem Cell Therapy for Stroke Patients: Are We There Yet?”, 8:983-988 (2019), DOI: 10.1002/sctm.19-0076; in Connor, B., “Concise Review: The Use of Stem Cells for Understanding and Treating Huntington's Disease”, Stem Cells, 36:146-160 (2017), DOI: 10.1002/stem.2747; and in Grade, S. and Gotz, M., “Neuronal replacement therapy: previous achievements and challenges ahead”, npj Regenerative Medicine, vol. 2, Article No. 29 (2017), DOI: 10.1038/s41536-017-0033-0, the disclosures of which are incorporated herein in its entirety for all purposes.

In some embodiments, the target gene is selected from one or more of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz. In some embodiments, more than one target gene is selected. In some embodiments at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the foregoing genes are selected. In some embodiments, the target gene is selected from one or more of ATF3, ATF4, CEBPG, CHOP, Lkb1, Lpin2, Mkk4, Mkk7, Nt5c1a, SSRP1, and SUPT16. In some embodiments, the target gene is selected from one or more of MAPK8IP3, Mkk7, and Snrk. In some embodiments, the target gene is selected from one or more of the 1, 2, 3, 4, 5, 6, 7, 8, 9, 10, 15, 20, 25, 30, 35, 40, 45, or 50 genes associated with the highest number of RBPMS+ cells/mm² in FIG. 6A. In some embodiments, the target genes selected comprise MAP2K7 and MAP2K4. In some embodiments, the target gene comprises Lkb1.

In some embodiments, the target gene is selected from one or more of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930. In some embodiments, more than one target gene is selected. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the foregoing genes are selected. In some embodiments, the target gene is selected from one or more of Fastkd5, Lhx6, Pawr, Rbbp7, Snrk, Stk10, Stradα, Tcf3, Tgif1, and Tie1. In some embodiments, the target gene is Snrk. In some embodiments, the target gene is selected from one or more of the 1, 2, 3, 4, 5, 6, 7, 8, 9, 10, 15, 20, 25, or 30 genes associated with the highest normalized regenerating axon density (AU) in FIG. 6C.

In some embodiments where more than one target gene is selected, a synergistic regenerative or cell (e.g., a neuron) survival effect can be achieved by selecting at least one target gene from ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz and selecting at least one other distinct target gene from Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930. In some embodiments, a synergistic effect can be effected by selecting one target gene that is associated with an increase in cell survival (i.e., neuroprotection) and another target gene that is associated with an increase in cell growth (i.e., neuroregeneration).

### Pharmaceutical Compositions

In some aspects, the present invention provides pharmaceutical compositions. To prepare the pharmaceutical compositions of this invention, an effective amount of an agent (e.g., small molecule, a vector, or an inhibitory polynucleotide) is combined with a pharmaceutically acceptable carrier, which carrier may take a wide variety of forms depending on the form of preparation desired for administration. In some embodiments, the pharmaceutical composition comprises a neuron or stem cell treated by any of the methods provided herein with any of the agents described herein to reduce or eliminate expression of a target gene and/or to inhibit or eliminate activity of a molecular target. In some embodiments, the agent is an inhibitor of a molecular target. These pharmaceutical compositions are desirable in unitary dosage form suitable, particularly, for administration percutaneously, or by parenteral injection. Any of the usual pharmaceutical media may be employed such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules, and tablets. For parenteral compositions, the carrier will usually comprise sterile water, at least in large part, though other ingredients, for example, to aid solubility and cell viability, may be included. Other ingredients may include antioxidants, viscosity stabilizers, chelating agents, buffers, preservatives. If desired, further ingredients may be incorporated in the compositions, e.g. anti-inflammatory agents, antibacterials, antifungals, disinfectants, vitamins, antibiotics.

The agent in various embodiments inhibits expression of a target gene or a molecular target associated with the target gene. In some embodiments, the target gene is selected from one or more of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz. In some embodiments, more than one target gene is selected. In some embodiments at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the foregoing genes are selected. In some embodiments, the target gene is selected from one or more of ATF3, ATF4, CEBPG, CHOP, Lkb1, Lpin2, Mkk4, Mkk7, Nt5c1a, SSRP1, and SUPT16. In some embodiments, the target gene is selected from one or more of MAPK8IP3, Mkk7, and Snrk. In some embodiments, the target gene is selected from one or more of the 1, 2, 3, 4, 5, 6, 7, 8, 9, 10, 15, 20, 25, 30, 35, 40, 45, or 50 genes associated with the highest number of RBPMS+ cells/mm² in FIG. 6A. In some embodiments, the target genes selected comprise MAP2K7 and MAP2K4. In some embodiments, the target gene comprises Lkb1.

In some embodiments, the target gene is selected from one or more of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930. In some embodiments, more than one target gene is selected. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the foregoing genes are selected. In some embodiments, the target gene is selected from one or more of Fastkd5, Lhx6, Pawr, Rbbp7, Snrk, Stk10, Stradα, Tcf3, Tgif1, and Tie1. In some embodiments, the target gene is Snrk. In some embodiments, the target gene is selected from one or more of the 1, 2, 3, 4, 5, 6, 7, 8, 9, 10, 15, 20, 25, or 30 genes associated with the highest normalized regenerating axon density (AU) in FIG. 6C.

In some embodiments where more than one target gene is selected, a synergistic regenerative or cell (e.g., a neuron) survival effect can be achieved by selecting at least one target gene from ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz and selecting at least one other distinct target gene from Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930. In some embodiments, a synergistic effect can be effected by selecting one target gene that is associated with an increase in cell survival (i.e., neuroprotection) and another target gene that is associated with an increase in cell growth (i.e., neuroregeneration).

The invention provides therapeutic compositions that decrease the expression of a target gene directly or indirectly or that inhibit activity of a molecular target (e.g., a polypeptide encoded by a target gene). In some embodiments, a decrease in expression of the target gene or inhibition of a molecular target results in increased survival, decrease in death of a neuron or protection from neuron degeneration. In some embodiments, a decrease in expression of the target gene or inhibition of a molecular target results in neuroregeneration of a neuron (e.g., regrowth of an axon). In one embodiment, the present invention provides a pharmaceutical composition comprising an inhibitory nucleic acid molecule (e.g., an antisense, siRNA, or shRNA polynucleotide) that decreases the expression of one or more target genes. In another embodiment, the present invention provides a pharmaceutical composition comprising a small molecule inhibitor of a molecular target. In some embodiments, the present invention provides a pharmaceutical composition comprising a heterologous gene sequence expressing a gene used in gene editing or CRISPR interference. Agents of the invention may be administered as part of a pharmaceutical composition. The compositions should be sterile and contain a therapeutically effective amount of the polypeptides or nucleic acid molecules in a unit of weight or volume suitable for administration to a subject.

Agents of the invention (e.g., an inhibitory nucleic acid molecule or a polynucleotide encoding a gene editing polynucleotide) may be administered within a pharmaceutically-acceptable diluent, carrier, or excipient, in unit dosage form. Conventional pharmaceutical practice may be employed to provide suitable formulations or compositions to administer the compounds to patients suffering from a neurological condition. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols. In some embodiments, the composition is administered locally to a patient (e.g., at a site of injury) and not systemically. In some embodiment, the composition is administered systemically.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for inhibitory nucleic acid molecules include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. The formulations can be administered to human patients in therapeutically effective amounts (e.g., amounts which prevent, eliminate, or reduce a pathological condition) to provide therapy for a neoplastic disease or condition. The preferred dosage of an agent of the invention is likely to depend on such variables as the type and extent of the disorder, the overall health status of the particular patient, the formulation of the compound excipients, and its route of administration.

With respect to a subject having a neurological condition, an effective amount is sufficient to ameliorate the neurological condition. Generally, doses of agent of the present invention would be from about 0.01 mg/kg per day to about 1000 mg/kg per day. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of an agent of the invention.

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes.

### Methods of Treatment

The present invention provides methods of treating disease, injury, and/or disorder or symptoms thereof, where the methods involve administering a therapeutically effective amount of a pharmaceutical composition of the present disclosure to a subject (e.g., a mammal such as a human). In an embodiment, the method includes the step of administering to a subject a therapeutic amount of an agent described herein sufficient to treat the disease, injury, or disorder or a symptom thereof, under conditions such that the disease or disorder is treated. In some embodiments, the composition is a pharmaceutical composition described herein.

The described method has wide applicability to the treatment of central nervous system (CNS) degeneration/damage. In some embodiments, the central nervous system degeneration/damage is caused by a neurodegenerative disease or CNS trauma/injury. In this regard, the subject method is useful for, but not limited to, treatment of injury to the brain and spinal cord due to ischemias, hypoxia, traumas, neurodegenerative diseases, infectious diseases, cancers, autoimmune diseases and metabolic disorders. Examples of disorders include stroke, aneurism, head trauma, spinal trauma, hypotension, arrested breathing, cardiac arrest, Reye's syndrome, cerebral thrombosis, embolism, cerebral hemorrhage, brain tumors, encephalomyelitis, hydroencephalitis, and operative and postoperative brain injury. Non-limiting examples of neurodegenerative diseases that may be treated using the compositions and/or methods of the present disclosure include Alzheimer's disease, Parkinson's disease, Huntington's disease, multiple sclerosis (MS), amyotrophic lateral sclerosis (ALS), glaucoma, metachromatic leuokodystrophy, adrenoleukodystophy, lysosomal storage disorders, traumatic brain injury, spinal cord injury, spinal cord crush, and/or optic nerve injury.

The methods herein include administering to the subject (including a subject identified as in need of such treatment) an effective amount of an agent described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention in general comprise administration of a therapeutically effective amount of the compositions described herein, such as a composition comprising a small molecule inhibitor or an inhibitory nucleic acid, to a subject (e.g., animal, human) in need thereof, including a mammal, particularly a human. Such treatment will be suitably administered to subjects, particularly humans, suffering from, having, susceptible to, or at risk for a disease, injury, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any injury, disorder, or disease resulting in neuron degeneration/damage.

The pharmaceutical compositions of this invention can be administered by any suitable routes including, by way of illustration, oral, topical, rectal, transdermal, subcutaneous, intravenous, intramuscular, intranasal, intracranial, intracerebral, intraventricular, intrathecal, and the like. In some embodiments, the administration modalities as described in U.S. Pat. Nos. 5,543,158; 5,641,515 and 5,399,363 may be used to deliver compositions of the present invention. In some embodiments, a preferred mode of administration is by portal vein injection.

For therapeutic uses, the compositions and agents disclosed herein may be administered by any convenient method; for example, parenterally, conveniently in a pharmaceutically or physiologically acceptable carrier, e.g., phosphate buffered saline, saline, deionized water, or the like. The compositions may be added to a retained physiological fluid such as blood or synovial fluid. For central nervous system (CNS) administration, a variety of techniques are available for promoting transfer of an agent across the blood brain barrier including disruption by surgery or injection, drugs which transiently open adhesion contact between central nervous system (CNS) vasculature endothelial cells, and compounds which facilitate translocation through such cells.

As examples, many of the disclosed compositions are amenable to be directly injected or infused or contained within implants e.g. osmotic pumps, grafts comprising appropriately transformed cells. Compositions of the present invention may also be amenable to direct injection or infusion, topical, intratracheal/nasal administration e.g. through aerosol, intraocularly, or within/on implants e.g. fibers e.g. collagen, osmotic pumps, or grafts comprising appropriately transformed cells. Generally, the amount administered will be empirically determined. Other additives may be included, such as stabilizers, bactericides, etc. In various embodiments, these additives can be present in conventional amounts.

In various embodiments, the agents of the present invention are administered in sufficient amounts to provide sufficient levels of the agent in a target cell without undue adverse effects. Conventional and pharmaceutically acceptable routes of administration include, but are not limited to, direct delivery to a selected organ or tissue (e.g., the spinal cord or brain), oral, inhalation (including intranasal and intratracheal delivery), intraocular, intravenous, intramuscular, subcutaneous, intradermal, intratumoral, and other parental routes of administration. Routes of administration may be combined, if desired.

The dose of an agent used to achieve a particular “therapeutic effect” will vary based on several factors including, but not limited to: the route of administration, the level of gene or RNA expression used to achieve a therapeutic effect, the specific disease or disorder being treated, and the stability of the gene or RNA product. One of skill in the art can readily determine a dose range to treat a patient having a particular disease, injury, or condition based on the aforementioned factors, as well as other factors that are well known in the art. In some embodiments, the therapeutic effect is axon regeneration, inhibition of cell death, protection from neuron degeneration or restoration of neuronal cell health.

Administration of agents of the present invention to a subject may be by, for example, intramuscular injection or by administration into the bloodstream of the subject. Administration into the bloodstream may be by injection into a vein, an artery, or any other vascular conduit. In some embodiments, the agents are administered into the bloodstream by way of isolated limb perfusion, a technique well known in the surgical arts, the method essentially enabling the artisan to isolate a limb from the systemic circulation prior to administration. A variant of the isolated limb perfusion technique, described in U.S. Pat. No. 6,177,403, can also be employed by the skilled artisan to administer the agent into the vasculature of an isolated limb to potentially enhance transduction into muscle cells or tissue. Moreover, in certain instances, it may be desirable to deliver the agent to the central nervous system (CNS) of a subject. In various embodiments, by “CNS” is meant all cells and tissue of the brain and spinal cord of a vertebrate. Thus, the term can include, but is not be limited to, neuronal cells, glial cells, astrocytes, cereobrospinal fluid (CSF), interstitial spaces, bone, cartilage and the like. An agent may be delivered directly to the central nervous system (CNS) or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection.

Agents of the present invention can be inserted into a delivery device which facilitates introduction by injection or implantation into a subject. Such delivery devices include tubes, e.g., catheters, for injecting cells and fluids into the body of a recipient subject. In a preferred embodiment, the tubes additionally have a needle, e.g., a syringe, through which the contents of the invention can be introduced into the subject at a desired location. Agents of the invention can be inserted into such a delivery device, e.g., a syringe, in different forms. For example, an agent can be suspended in a solution or embedded in a support matrix when contained in such a delivery device. As used herein, the term “solution” includes a pharmaceutically acceptable carrier or diluent in which the agent of the invention remain functional and/or viable. Pharmaceutically acceptable carriers and diluents include saline, aqueous buffer solutions, solvents and/or dispersion media. The use of such carriers and diluents is well known in the art. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. In some embodiments, the selection of the carrier is not a limitation of the present invention. The solution is preferably sterile and fluid. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating recombinant adeno-associated virus particles, nucleotide molecules, and/or vectors as described herein in a pharmaceutically acceptable carrier or diluent and, as other ingredients enumerated herein, followed by filtered sterilization. Optionally, an agent may be administered on support matrices. Support matrices in which an agent can be incorporated or embedded include matrices which are recipient-compatible and which degrade into products which are not harmful to the recipient. Natural and/or synthetic biodegradable matrices are examples of such matrices. Natural biodegradable matrices include plasma clots, e.g., derived from a mammal, and collagen matrices. Synthetic biodegradable matrices include synthetic polymers such as polyanhydrides, polyorthoesters, and polylactic acid. Other examples of synthetic polymers and methods of incorporating or embedding cells into these matrices are known in the art. These matrices provide support and protection for the cells in vivo.

In some embodiments, methods are provided for implanting a neuron or stem cell treated by any of the survival- and/or growth-promoting methods provided herein and/or contacted with any of the agents described herein to reduce or eliminate expression of a target gene and/or to inhibit or eliminate activity of a molecular target into a subject. Methods for transplantation of neurons into a subject are known in the art and can be found, for example, in Ishikawa, M., et al., “Transplantation of neurons derived from human iPS cells cultured on collagen matrix into guinea-pig cochleae”, Journal of Tissue Engineering and Regenerative Medicine, 11:1766-1778 (2017), DOI: 10.1002/term.2072; and in Derr, J. et al, “Whole-brain 3D mapping of human neural transplant innervation”, Nature Communications, vol. 8, Art. No. 14162 (2017), DOI: 10.1038/ncomms14162; and Kondziolka, D., et al., “Transplantation of cultured human neuronal cells for patients with stroke”, Neurology, vol. 55, DOI: https://doi.org/10.1212/WNL.55.4.565; and Harward, S., et al, “Interneuron transplantation: a prospective surgical therapy for medically refractory epilepsy”, Journal of Neurosurgery, 48:E18 (2020), DOI: 10.3171/2020.2.FOCUS19955, the disclosures of which are incorporated herein by reference in their entirety for all purposes.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a bioactive factor at a particular target site.

One feature of certain embodiments of an implant can be the linear release of an agent of the present invention, which can be achieved through the manipulation of the polymer composition and form. By choice of monomer composition or polymerization technique, the amount of water, porosity and consequent permeability characteristics can be controlled. The selection of the shape, size, polymer, and method for implantation can be determined on an individual basis according to the disorder, injury, or disease to be treated and the individual patient response. The generation of such implants is generally known in the art.

In another embodiment of an implant an agent of the invention is encapsulated in implantable hollow fibers or the like. Such fibers can be pre-spun and subsequently loaded with the agent, or can be co-extruded with a polymer which acts to form a polymeric coat about the agent.

In addition to the methods of delivery described above, the following techniques are also contemplated as alternative methods of delivering an agent to a subject. Ultrasound has been used as a device for enhancing the rate and efficacy of drug permeation into and through a circulatory system. Other drug delivery alternatives contemplated are intraosseous injection (see, e.g., U.S. Pat. No. 5,779,708), microchip devices (see, e.g., U.S. Pat. No. 5,797,898), ophthalmic formulations, transdermal matrices (see, e.g., U.S. Pat. Nos. 5,770,219 and 5,783,208), and feedback-controlled delivery (see, e.g., U.S. Pat. No. 5,697,899).

### Kits

The invention provides kits for the treatment or prevention of a disease, injury, or disorder. The agents described herein may, in some embodiments, be assembled into pharmaceutical or diagnostic or research kits to facilitate their use in therapeutic, diagnostic or research applications. In certain embodiments agents in a kit may be in a pharmaceutical formulation and dosage suitable for a particular application and for a method of administration of the agents. Kits for research purposes may contain the components in appropriate concentrations or quantities for running various experiments.

Kits may include dose-size-specific ampules or aliquots of compositions of the present invention. Kits may also contain devices to be used in administering the compositions. In some embodiments, the kit comprises a sterile container which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The kit may be designed to facilitate use of the methods described herein. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or another suitable solvent), which may or may not be provided with the kit.

The kit may contain any one or more of the components described herein in one or more containers. As an example, in one embodiment, the kit may include instructions for mixing one or more components of the kit and/or isolating and mixing a sample and administering to a subject. The kit may include a container housing agents described herein. The agents may be in the form of a liquid, gel or solid (powder). The agents may be prepared sterilely, packaged in syringe and shipped refrigerated. A second container may comprise other agents prepared sterilely. Alternatively, the kit may include agents premixed and shipped in a syringe, vial, tube, or other container. The kit may have one or more or all of the components useful to administer the agents to a subject, such as a syringe, topical application devices, or intravenous needle tubing and bag.

If desired an agent of the invention is provided together with instructions for administering an agent of the present invention to a subject having or at risk of developing a disease, injury, or disorder described herein. The instructions will generally include information about the use of the composition for the treatment or prevention of the disease, injury, or disorder. In other embodiments, the instructions include at least one of the following: description of the therapeutic agent; dosage schedule and administration for treatment or prevention of a disease, injury, or disorder described herein; precautions; warnings; indications; counter-indications; overdosage information; adverse reactions; animal pharmacology; clinical studies; and/or references. The instructions may be printed directly on the container (when present), provided on a transportable storage medium, stored on a remote server, or provided as a label applied to the container, or as a separate sheet, pamphlet, card, or folder supplied in or with the container. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

In certain aspects, practitioners of the present invention may employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes 1 and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1. Forward Genetic CRISPR Screening Strategy in the Mouse Optic Nerve Crush (ONC) Model

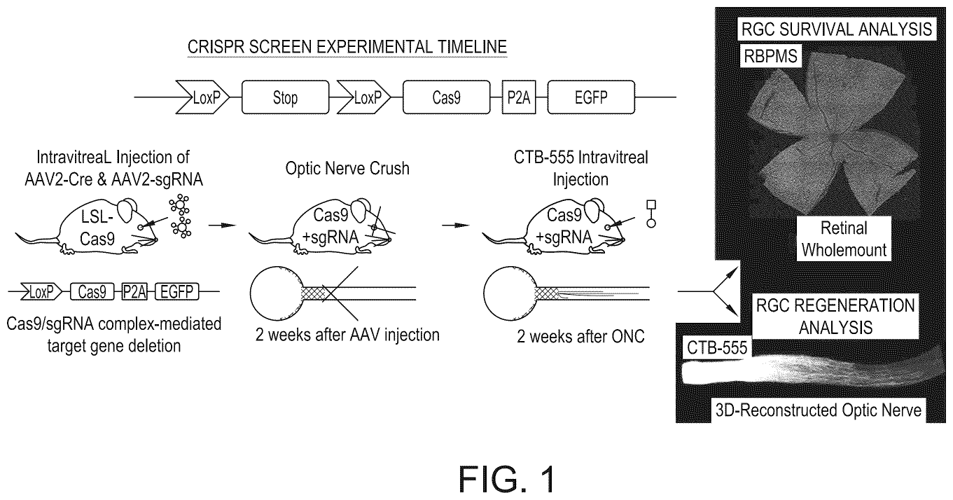

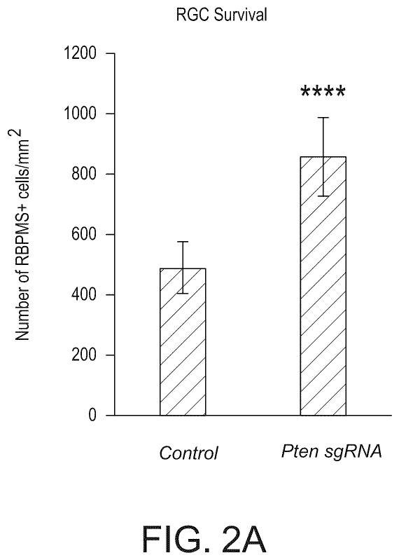

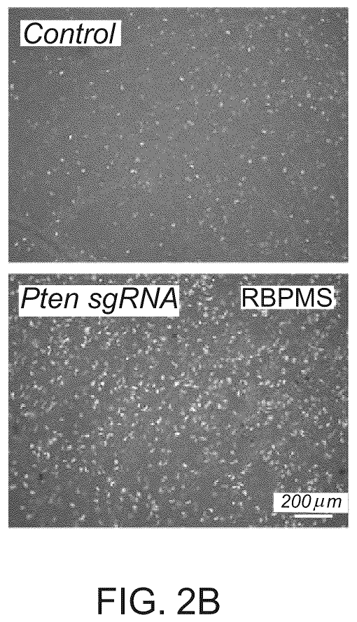

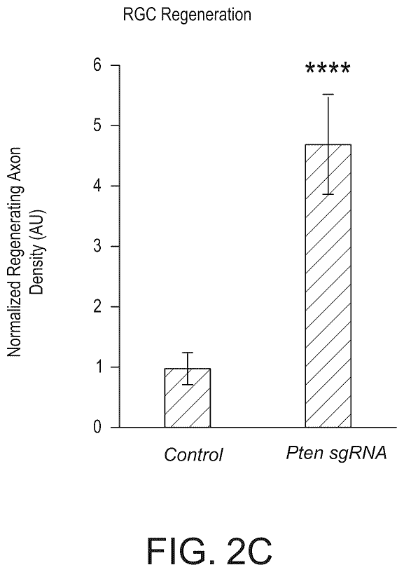

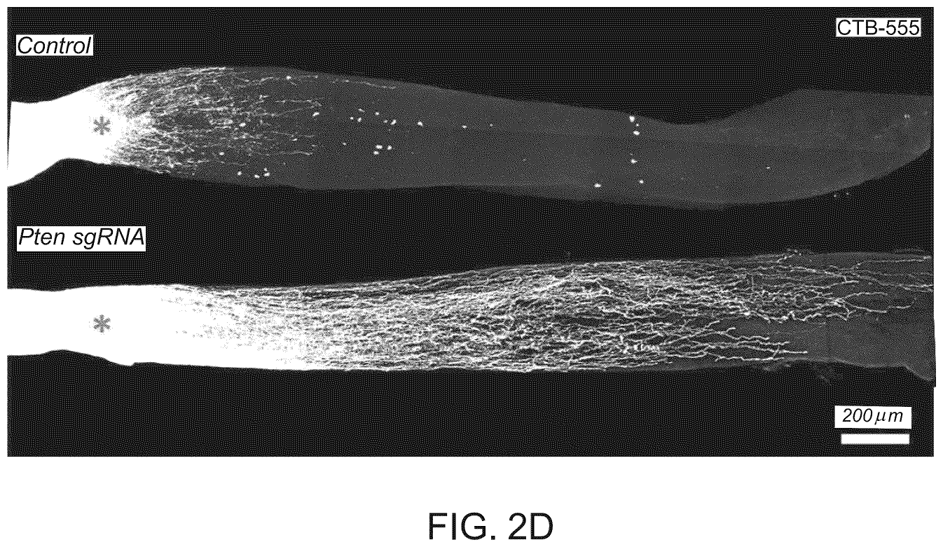

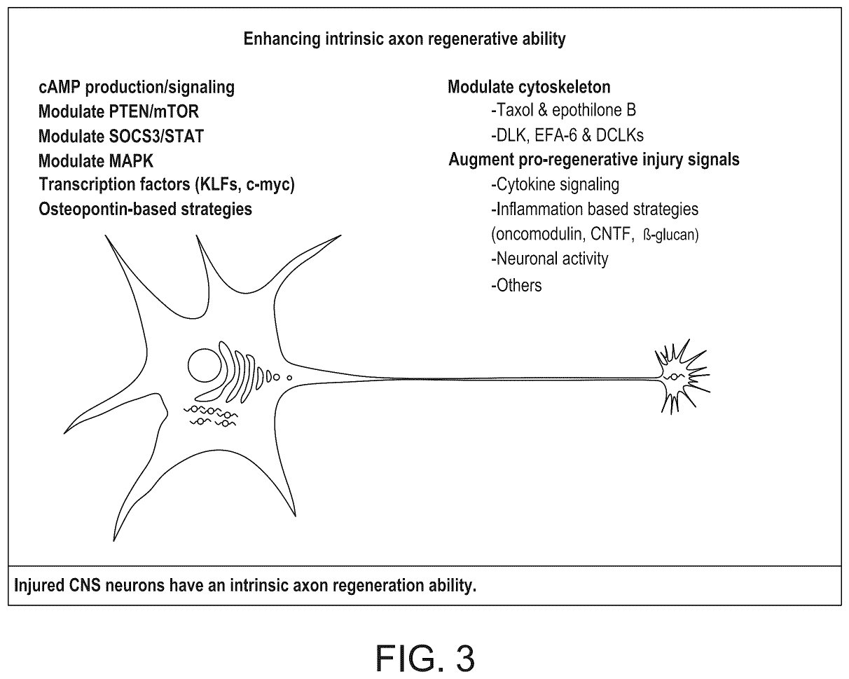

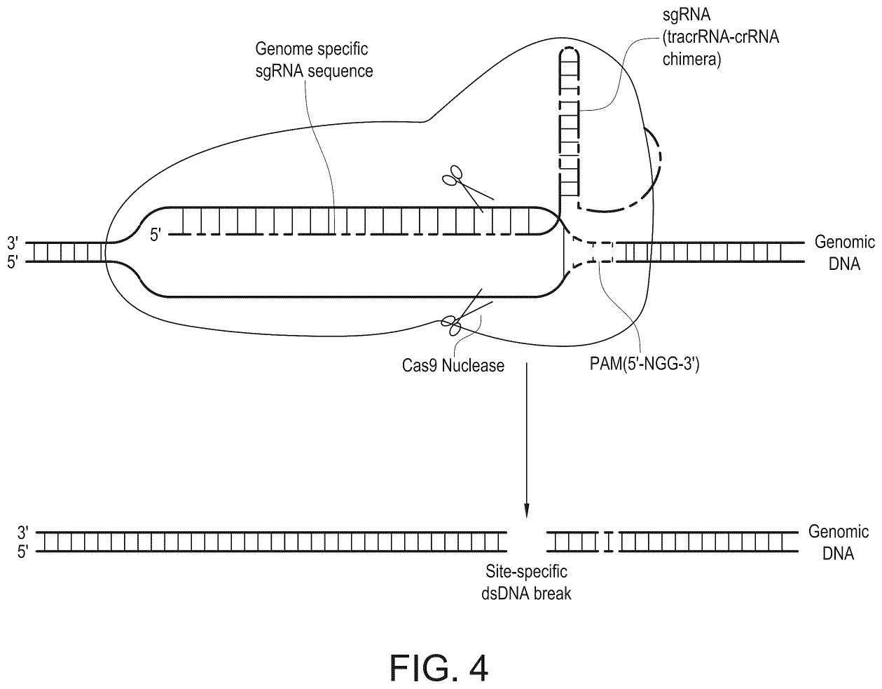

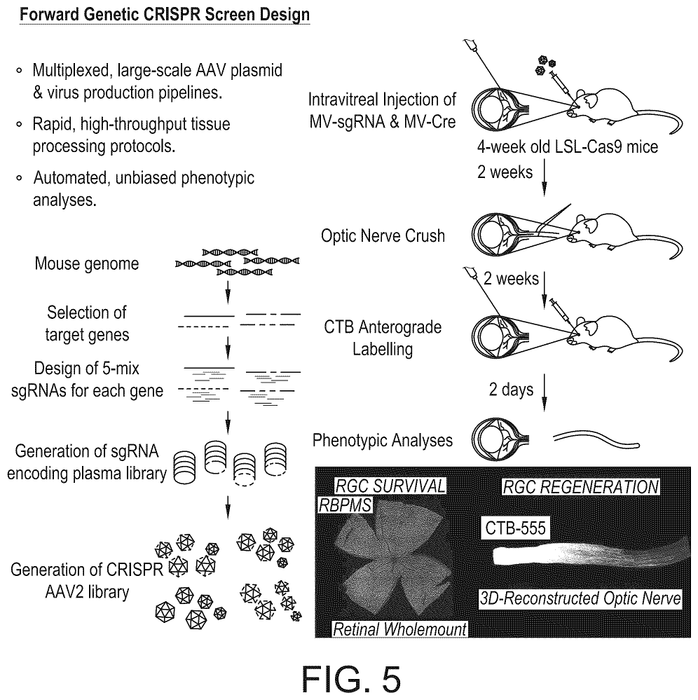

In order to conduct a forward genetic screen of transcription factor, kinome, phosphatome and other genes in vivo, a model system was used to maximize screening fidelity and throughput. To assess neuron survival and regeneration in a mammalian CNS neurodegeneration model, the relatively simple and reliable optic nerve crush (ONC) model was chosen, which causes ˜80% retinal ganglion cell (RGC) death at 2 weeks after injury, and displays no spontaneous retinal ganglion cell (RGC) axon regeneration (Tran, N. M., Shekhar, K., Whitney, I. E., Jacobi, A., Benhar, I., Hong, G., et al. (2019). Single-Cell Profiles of Retinal Ganglion Cells Differing in Resilience to Injury Reveal Neuroprotective Genes. Neuron 104, 1039-1055.e12. doi:10.1016/j.neuron.2019.11.006). This animal model has been used to demonstrate neuroprotection and neuroregeneration effects in degenerating central nervous system (CNS) neurons, and, particularly, that certain gene manipulations can robustly induce neuronal survival and axon regeneration after CNS injury (see, e.g., FIG. 3). Next, CRISPR/Cas9 technology, see FIG. 4, was utilized to delete target genes in retinal ganglion cells (RGCs). Only a small proportion of genes have been investigated in this context due to the considerable time and costs required for studies in conditional knockout (cKO) mice. To achieve this, a library of sgRNA plasmids were generated targeting transcription factor, kinome, phosphatome and other genes (>2,000 genes), based on the gRNA sequences from Zhang Feng's mouse Genome-scale CRISPR Knock-Out (GeCKO) library (Sanjana, N. E., Shalem, O., and Zhang, F. (2014). Improved vectors and genome-wide libraries for CRISPR screening. Nat. Methods 11, 783-784. doi:10.1038/nmeth.3047; see also Horlbeck, et al. Compact and highly active next-generation libraries for CRISPR-mediated gene repression and activation. eLife. DOI: 10.7554/eLife.19760) (see Table 2). To efficiently transduce retinal ganglion cells (RGC), AAV2 viruses were manufactured with sgRNA for each target gene, as intravitreally-injected AAV2 viruses are highly effective at targeting retinal ganglion cells (RGC). The production of >2,000 plasmids and >2,000 viruses for the screen was expedited by introducing a number of multiplexing steps and industrial scale solutions to their respected production pipelines, see FIG. 5. For target gene deletion, Rosa-Lox-Stop-Lox-Cas9-EGFP knock-in (LSL-Cas9) mice were intravitreally co-injected with AAV2-Cre and AAV2-sgRNA (targeting gene-of-interest) 2 weeks before optic nerve crush (ONC), to provide sufficient time for adeno-associated virus (AAV) life cycle completion, CRISPR knockout (KO), and extant protein turnover. Retinal ganglion cell (RGC) survival and axon regeneration were then assessed 2 weeks after optic nerve crush (ONC), following anterograde labelling of retinal ganglion cells (RGC) by intravitreal injection of Cholera Toxin Subunit B (CTB)-555 (FIG. 1). Because each AAV-CRISPR-mediated gene deletion was tested n=1, the following exclusion criteria were applied: (1) Poor eyeball health (e.g. cataracts or microphthalmia); (2) Lens injury (due to risk of a false positive); and (3) Low GFP expression (indicative of poor viral transduction). The following inclusion criteria were generally used to select target genes of interest: (1) survival increase of >25% retinal ganglion cells (RCGs) and/or (2) regenerating RGC axons detected at 500 μm from optic nerve crush (ONC) site. In some instances, particular hit genes qualitatively demonstrating neuroregenerative or neuroprotective activity, yet falling short of meeting one or more of these inclusion criteria, were nevertheless selected for inclusion and considered as meeting the inclusion criteria. Target genes meeting inclusion criterion (1) are referred to as neuroprotective target genes and target genes meeting inclusion criterion (2) are referred to as neuroregeneration target genes. This approach was validated using Pten sgRNA as a positive control, while injection of AAV2-Cre only was chosen as the negative control (FIGS. 2A-2D). In conclusion, this optimized CRISPR screening platform in the mouse optic nerve crush (ONC) model allowed the efficient testing of ˜50 CRISPR-mediated gene manipulations per week, with an experimental timeline of ˜5-6 weeks. Such throughput requires the initiation of the screening pipeline on ≥25 new LSL-Cas9 mice each week, prior production of AAVs, and running this cycle of experiments in parallel. In some instances, a mix of four genes were tested per eye and, if one of the genes met the inclusion criteria, each of the four genes was then re-tested singly.

TABLE 2
Single-guide RNA (sgRNA) gene library.

| Sub-library | Genes |
|---|---|
| Kinases, Phosphatases, Drug Targets | 2318 |
| Cancer and Apoptosis | 2916 |
| Stress and Proteostasis | 3093 |
| Mitochondria, Trafficking, Motility | 2219 |
| Gene Expression | 2292 |
| Membrane Proteins | 2418 |
| Unassigned | 3649 |

For an unbiased assessment of the effect of each of the AAV-CRISPR-mediated gene deletions on retinal ganglion cell (RGC) neuron survival at 2 weeks after ONC, retina whole mounts were immunostained for a pan-RGC (retinal ganglion cell) marker, RNA-binding protein with multiple splicing (RBMPS), and the number of surviving RBPMS+RGCs were automatically counted, and then quantified by determining the number of RBMPS+RGCs per mm² and comparing to control. For visualization of regeneration of the retinal ganglion cell (RGC) neurons, retinal ganglion cell (RGC) axons were anterogradely labeled by intravitreal injection of CTB-555 two days before the mice were transcardially perfused. To facilitate screening, a procedure was optimized for unbiased assessment of neuron regeneration in up to 64 optic nerves per week. This involved a rapid tissue clearing protocol (<48 hours) within a 96-well plate, confocal microscope-mediated optical sectioning of the cleared nerves, and automated quantification of CTB-555-labelled RGC axon density within the 3D-reconstructed images of these optic nerves.

### Example 2. Identification of Novel, Clinically-Translatable Gene Targets for Promoting Neuronal Survival and Axon Regeneration after a Central Nervous System (CNS) Nerve Injury Using CRISPR/Cas9 Gene-Editing System and the Adult Mouse Optic Nerve Crush (ONC) Model

Neuron degeneration occurs as a result of neuronal injury and neurodegenerative disease in the mammalian central nervous system (CNS). Neuron degeneration involves both degeneration of the axon, and degeneration of the cell body (soma) and the other neurites of the neuron. In order to treat neurodegenerative diseases and CNS injuries, it is necessary to identify ways to protect the central nervous system (CNS) neuron from such degeneration, and regenerate the neuron if it has already undergone degeneration. While molecules have been identified as targets for neuroprotection and neurodegeneration previously, only a subset of these therapeutic targets have been tested in animal models to date due to the limitations and difficulties associated with generating mice with genetic modifications. Using CRISPR-Cas9 gene editing, as described above, can overcome these historical problems, allowing genome-wide screening. By conducting an unbiased, in vivo CRISPR screen in an animal model of CNS neuron degeneration, several molecules were identified that can significantly promote neuroprotection and/or neuroregeneration of degenerating CNS neurons. Two weeks after ONC, approximately 70-80% of RGC neurons degenerate and die. These RGCs do not spontaneously regenerate their injured axons in the optic nerve. AAV-CRISPR-mediated deletion of genes in the transcription factor, kinase and phosphatase categories, identified a number of ‘hit’ molecules that can significantly induce RGC survival and/or axon regeneration two weeks after ONC.

The above-described forward genetic CRISPR screening strategy in the mouse optic nerve crush (ONC) model was used to identify clinically-translatable gene targets for promoting neuronal survival and/or axon regeneration in a widely-used mouse model of CNS neuron degeneration. Identification of such gene targets is necessary because, for example, disruption of axonal connections leads to functional deficits after central nervous system (CNS) injuries and in neurodegenerative diseases, as CNS neurons fail to regenerate their axons after degeneration. The screen addressed the challenge that, although certain genetic manipulations may induce central nervous system (CNS) neurons to survive and regenerate after an axon injury, only a small subset of genes have been tested to date and considerable time and costs are associated with mice knockout (KO) studies. The screen also allowed for the identification of gene targets that are clinically translatable; for example, genes that are not associated with tumor formation (e.g., PTEN and c-Myc).

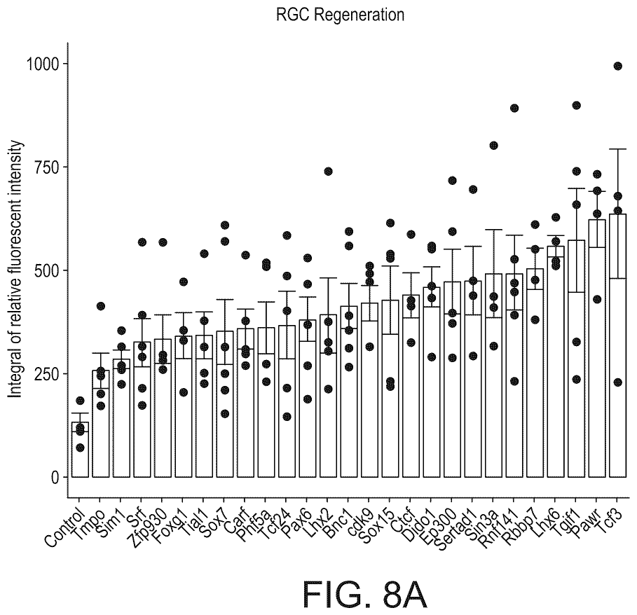

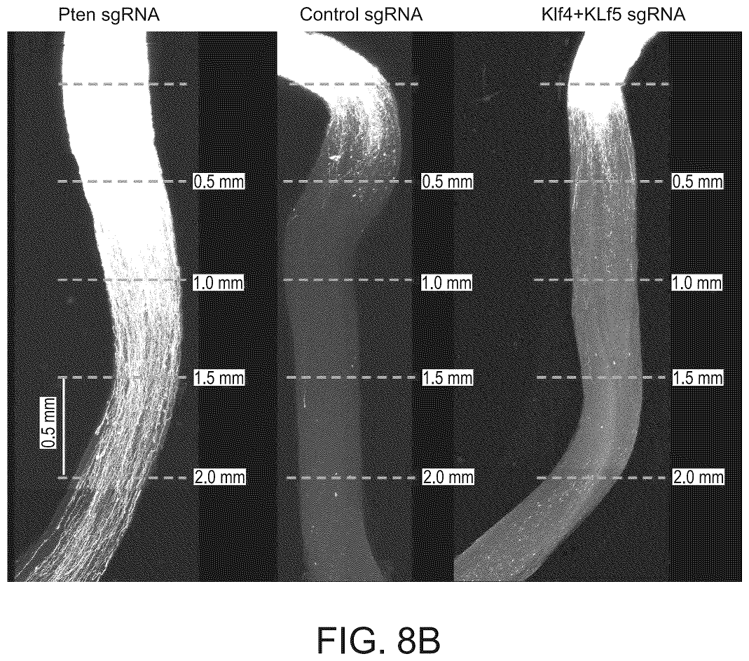

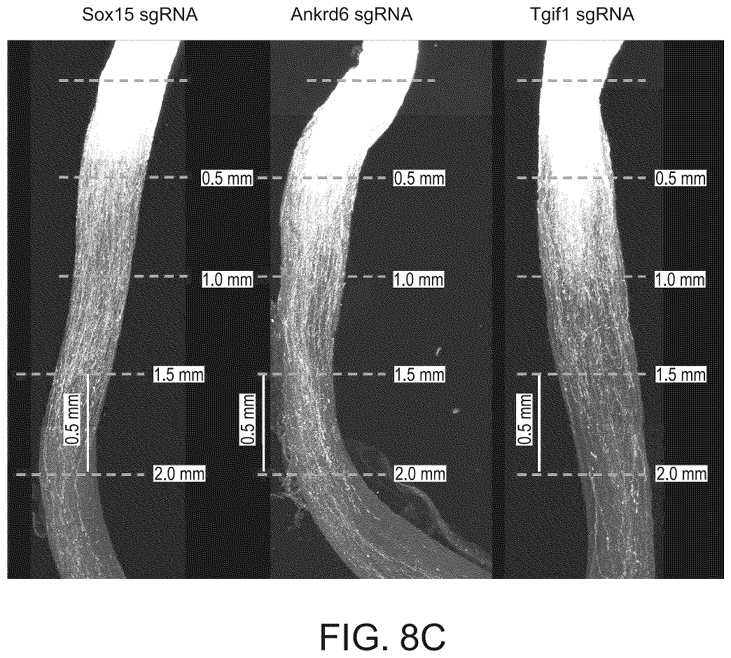

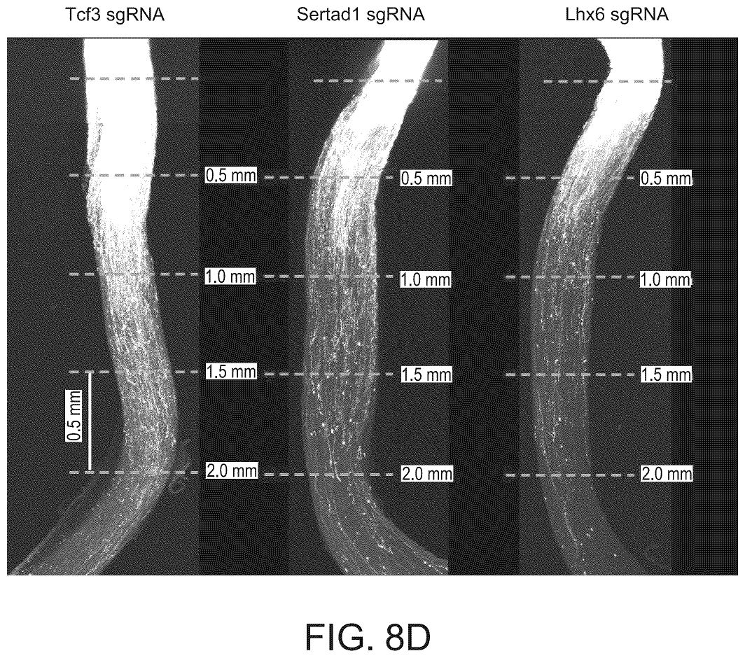

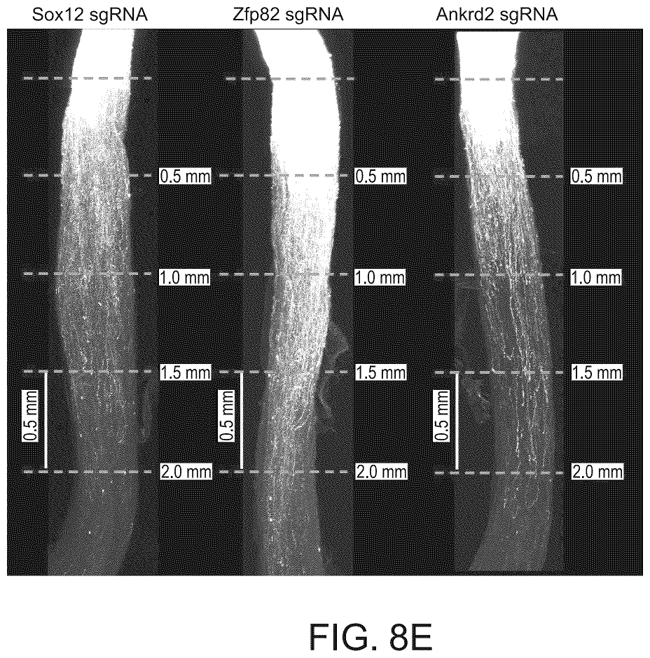

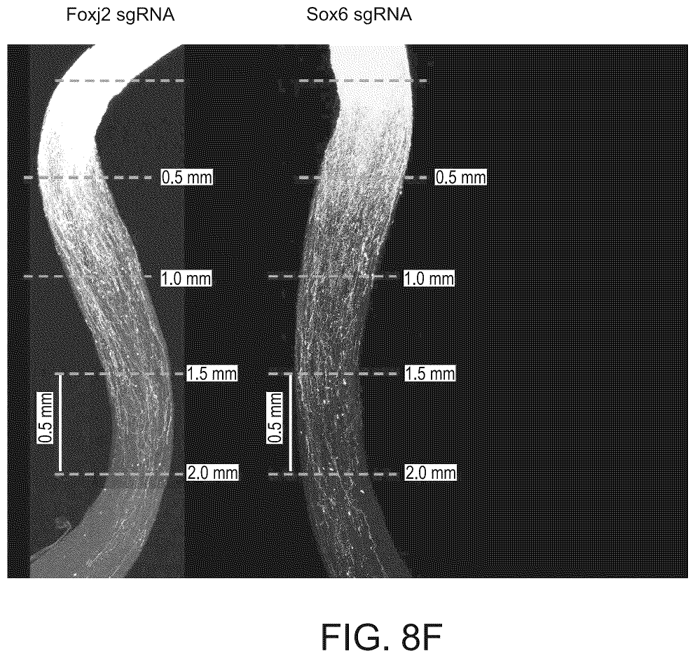

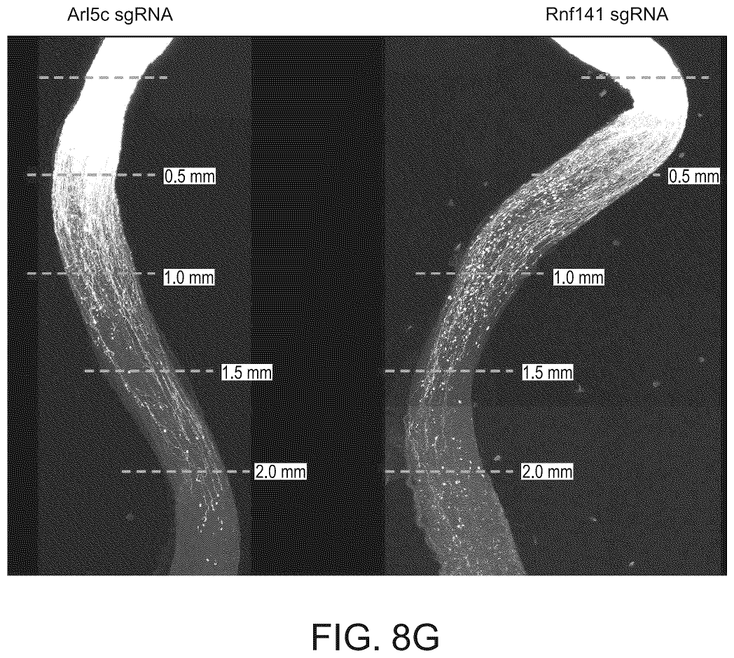

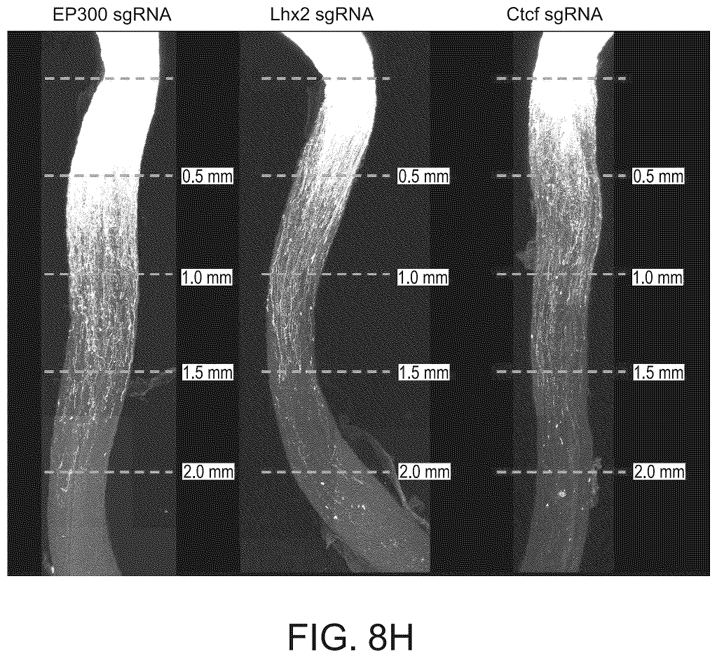

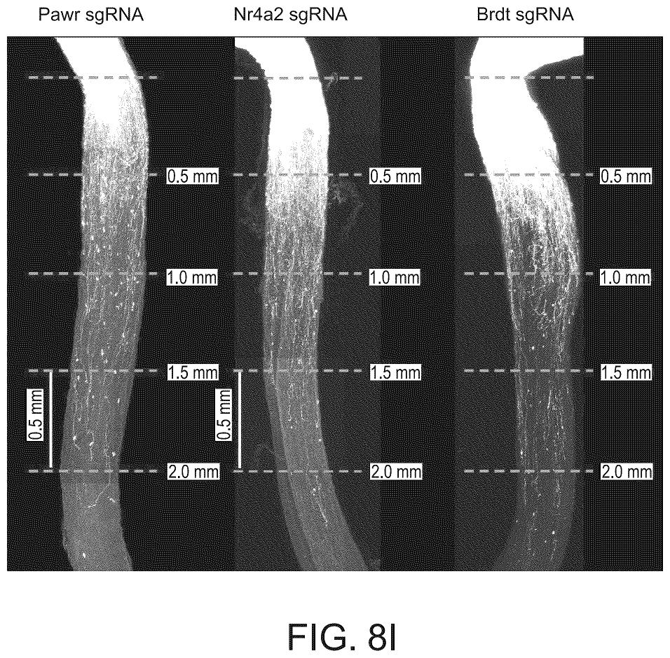

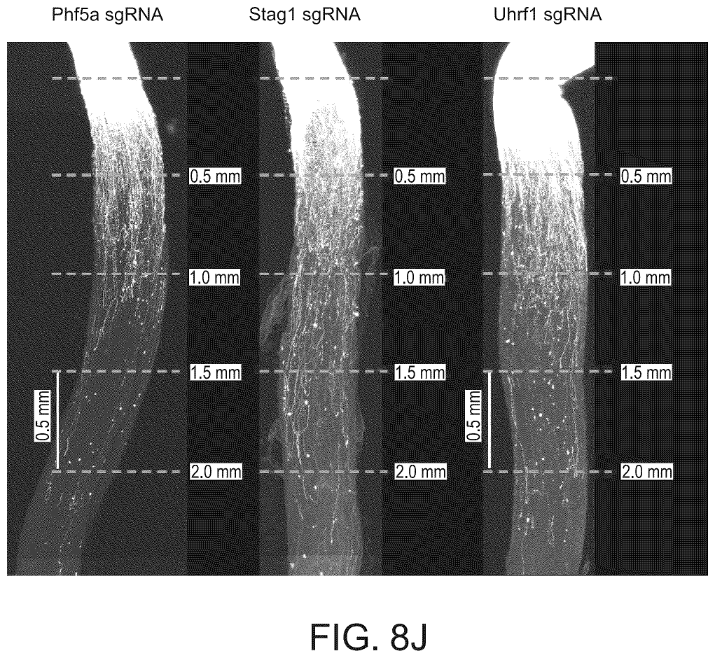

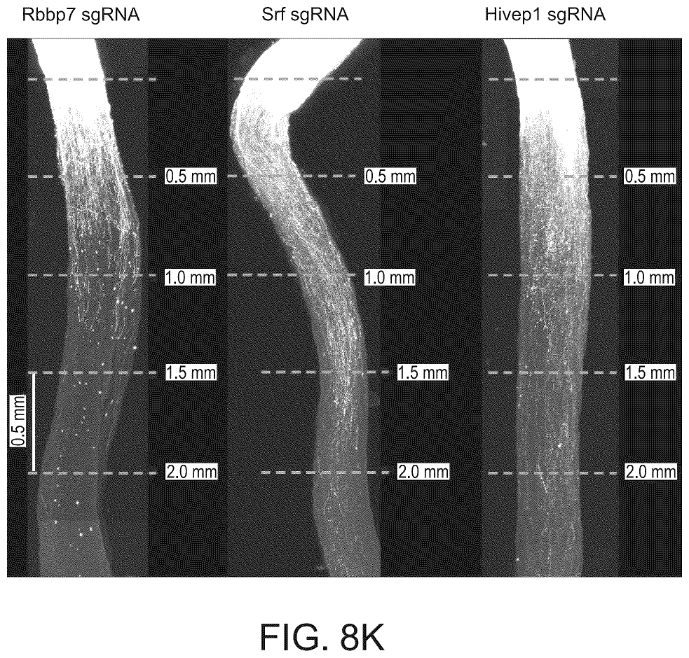

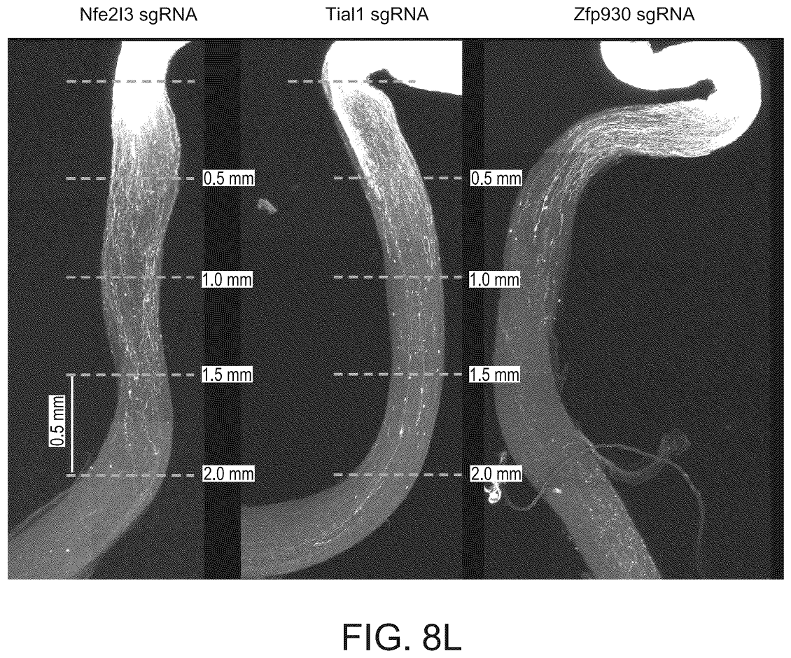

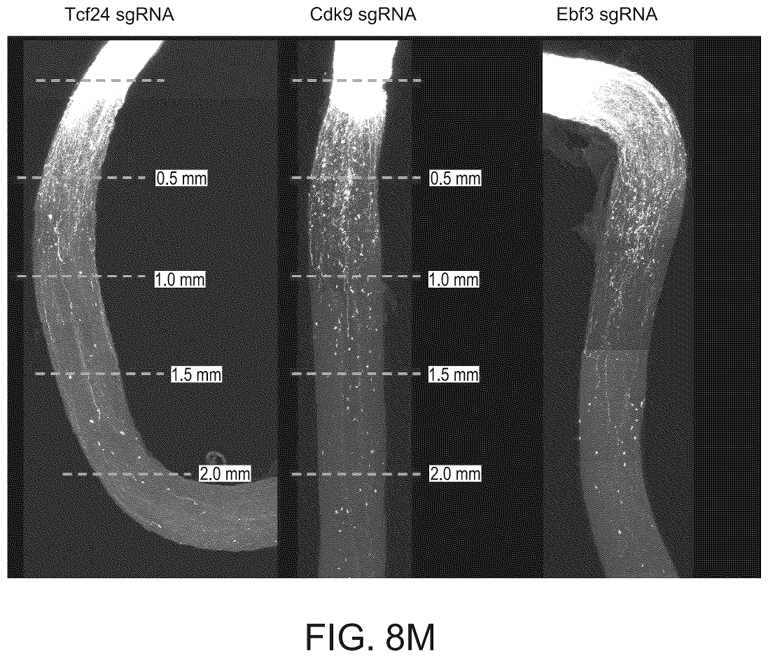

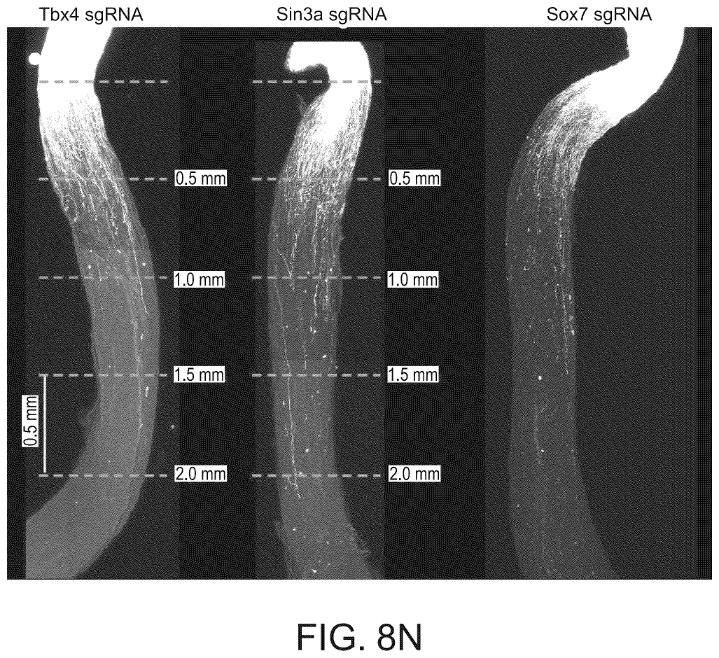

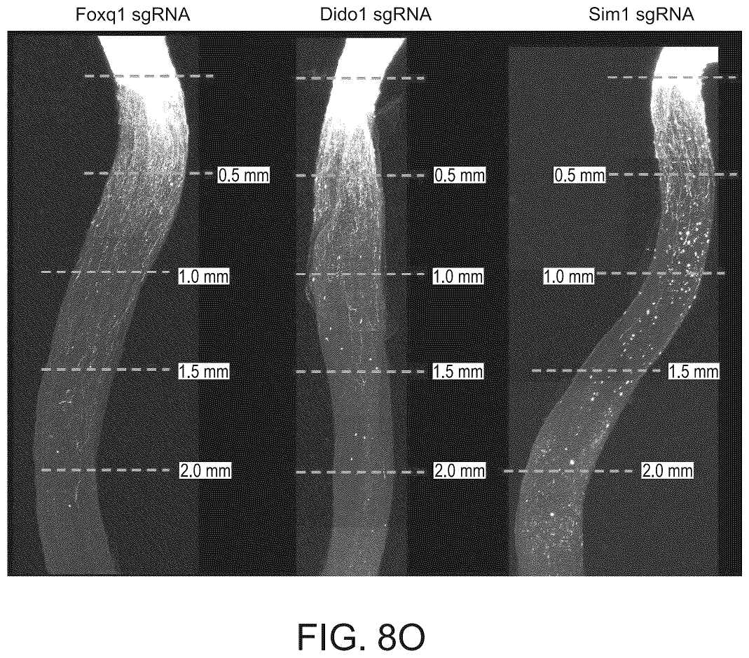

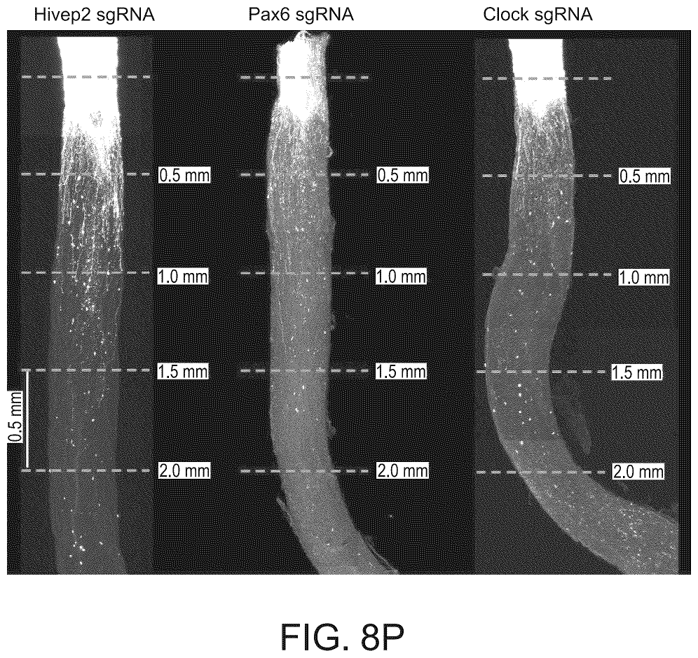

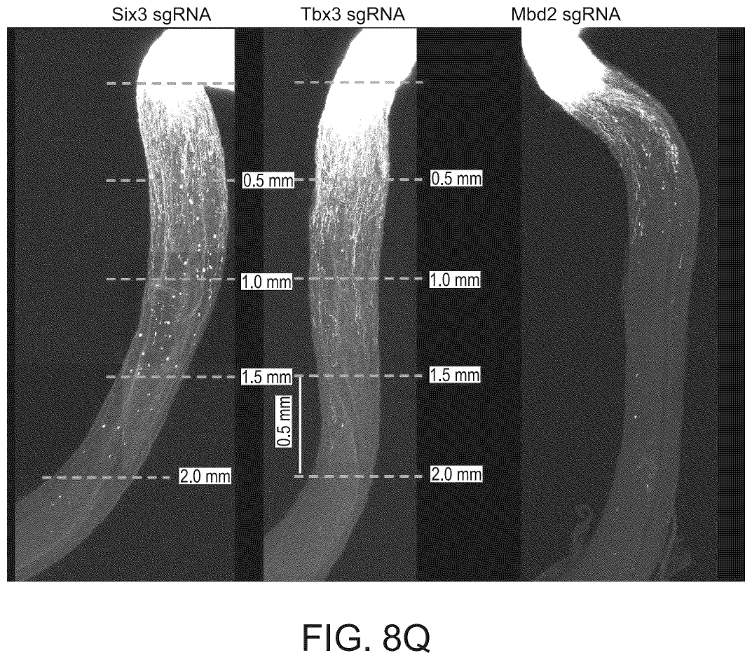

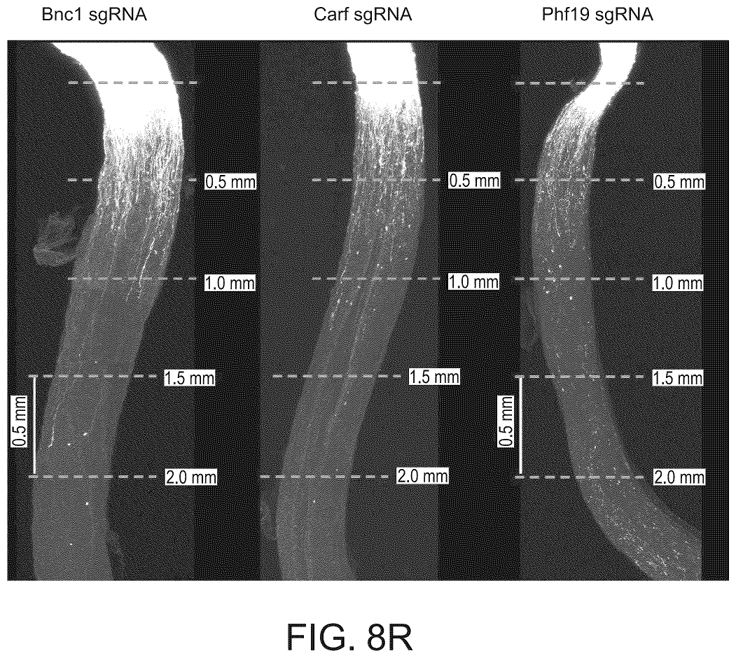

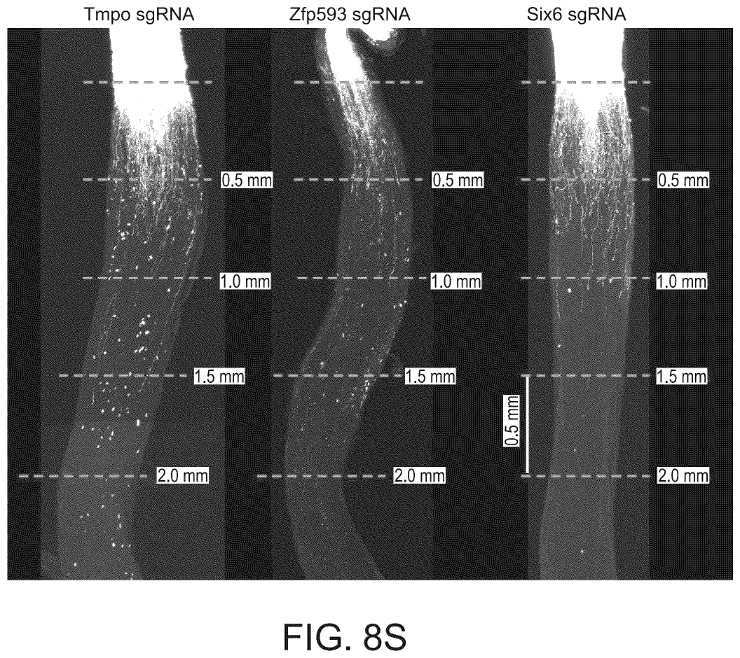

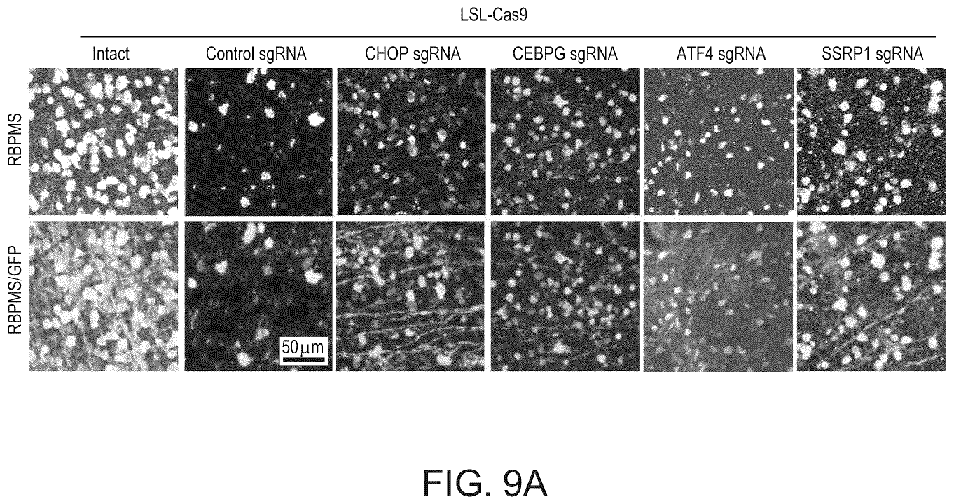

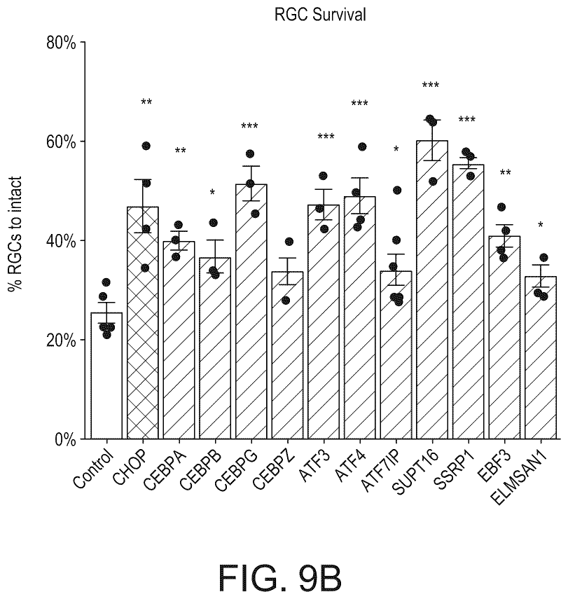

Of the dozens of genes meeting the inclusion criteria for retinal ganglion cell (RGC) survival, 63 significantly promoted retinal ganglion cell (RGC) survival, see FIGS. 6A, 6B, 9A and 9B. Neuroprotection hits identified included ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz. Of the dozens of genes meeting the inclusion criteria for retinal ganglion cell (RGC) regeneration, 31 significantly promoted retinal ganglion cell (RGC) regeneration, see FIGS. 6C, 6D, and 8A-8S. Neuroregeneration hits identified included Bnc1, Carf, cdk9, Ctef, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk1, Stradα, Tcf24, Tcf3, Tgif1, Tial1, Tie1, Tmpo, and Zfp930. The present invention in various embodiments is not limited by the classification of a gene target as belonging to a particular functional category (e.g., regulatory, transcription factor, kinase, or phosphatase).

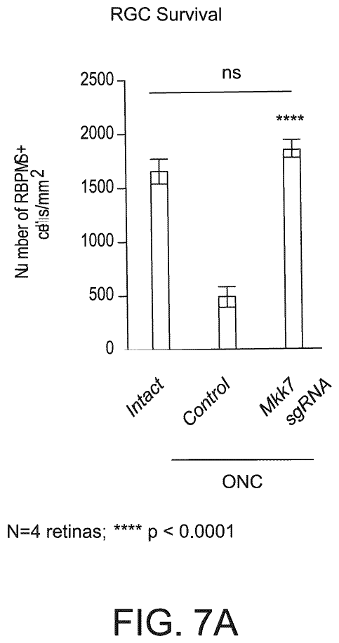

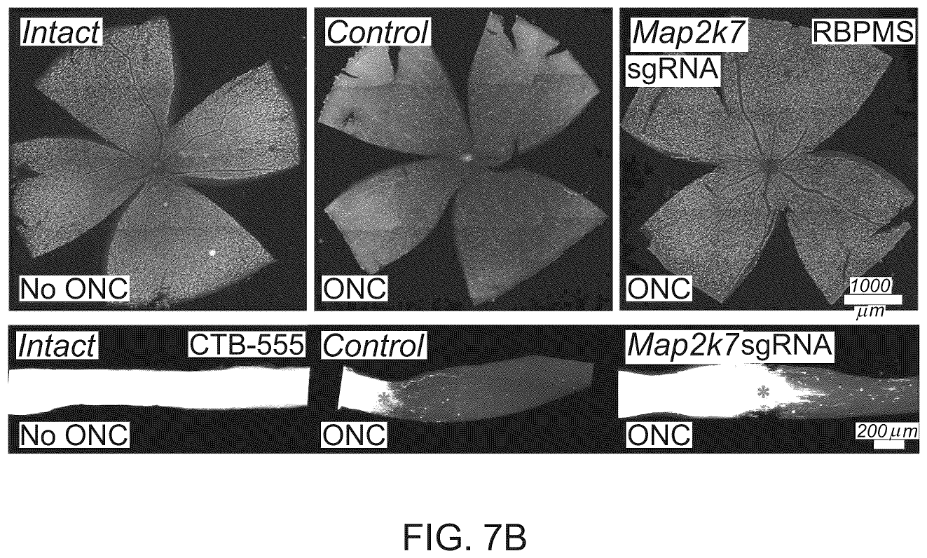

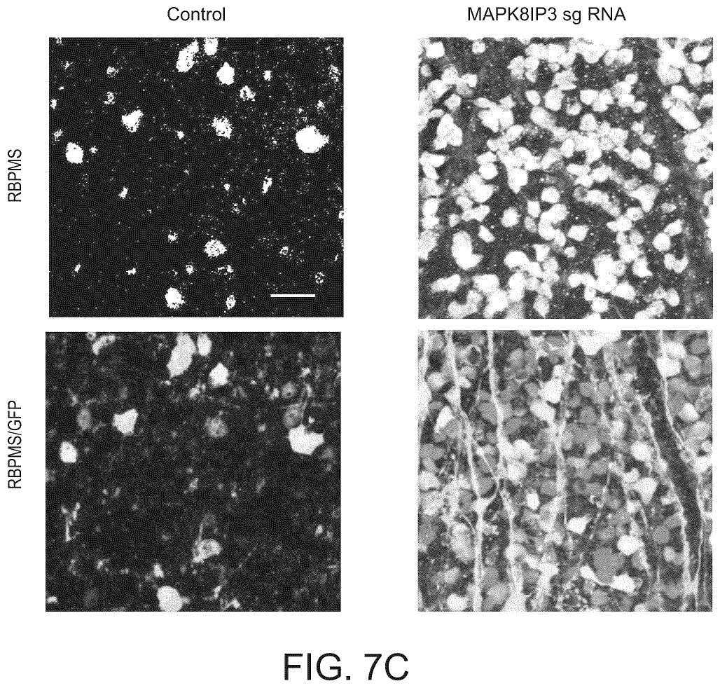

Of target genes identified promoting retinal ganglion cell survival, CRISPR knockout of MAP2K7 and MAPK8IP3 resulted in complete neuroprotection of central nervous system (CNS) neurons from degeneration after optic nerve crush injury, see FIGS. 7A-7C.

### Methods of the Examples

The following methods were employed in the above examples.

### Surgical Method for Intravitreal Injections and Optic Nerve Crush

All surgical procedures were performed in compliance with animal protocols approved by the IACUC at Boston Children's Hospital. For AAV injection, 4-week-old Lox-Stop-Lox-Cas9-EGFP knock-in (LSL-Cas9) mice were intravitreally-injected with 2-3 μl of either AAV-Cre (Control) and/or AAV-sgRNA (targeting gene-of-interest) with a pulled glass micropipette attached to a Hamilton syringe (Hamilton). For intravitreal injections, the pulled-glass micropipette was inserted near the peripheral retina behind the ora serrata and deliberately angled to avoid damage to the lens. Optic nerve crush (ONC) injury was performed two weeks after AAV injection, as per previously described (Park, K. K., Liu, K., Hu, Y., Smith, P. D., Wang, C., Cai, B., et al. (2008). Promoting axon regeneration in the adult CNS by modulation of the PTEN/mTOR pathway. Science (80-). 322, 963-966. doi:322/5903/963 [pii]10.1 126/science. 1161566 [doi]). Briefly, the optic nerve was exposed intraorbitally and crushed with a fine forceps (Dumont #5 FST) for 5 s approximately 500 mm behind the optic disc. Eye ointment was applied post-operatively to protect the cornea. At indicated time-points, mice were euthanized and optic nerves were immediately dissected out, and then flash frozen on dry ice.

### Optic Nerve Regeneration Visualization

For anterograde labeling of retinal ganglion cell axons within the optic nerve, 1 μl of cholera toxin subunit B with a conjugated Alexfluor555 (1 μg/μl in sterile saline, ThermoFisher) was injected intravitreally two days before perfusion. Dissected optic nerves were cleared using Visikol clearing solutions (per manufacturer's instructions). Cleared optic nerves anterogradely-labelled with CTB were then imaged using a Zeiss LSM 710 multiphoton confocal microscope.

### OTHER EMBODIMENTS

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for increasing survival, or reducing death or degeneration of a damaged or degenerating neuron, the method comprising contacting the damaged or degenerating neuron with an agent that reduces the expression or activity of a polypeptide selected from the group consisting of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz, thereby increasing survival, or reducing death or degeneration of the damaged or degenerating neuron.

2. The method of claim 1 further comprising contacting the damaged or degenerating neuron with another agent that reduces the expression or activity of an additional distinct polypeptide selected from the group consisting of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930, optionally to achieve a synergistic effect.

3. A method for increasing regeneration of a damaged or degenerating neuron, the method comprising contacting the damaged or degenerating neuron with an agent that reduces the expression or activity of a polypeptide selected from the group consisting of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930, thereby increasing regeneration of the damaged or degenerating neuron.

4. The method of claim 1, wherein damage to the neuron is associated with an injury or a neurodegenerative disease.

5. The method of claim 4, wherein the neurodegenerative disease is Alzheimer's disease, Parkinson's disease, Huntington's disease, multiple sclerosis (MS), amyotrophic lateral sclerosis (ALS), glaucoma, metachromatic leuokodystrophy, adrenoleukodystophy, or a lysosomal storage disorder.

6. The method of claim 4, wherein the injury is traumatic brain injury or spinal cord injury, traumatic brain injury, spinal cord injury, spinal cord crush, and/or optic nerve injury.

7. The method of claim 1, wherein the agent comprises a small molecule listed in Table 1, an inhibitory nucleic acid molecule comprising siRNA, shRNA, or antisense polynucleotide, or a CRISPR/Cas system.

8. A method for treating a damaged or degenerating neuron in a subject, the method comprising administering to the subject an agent that reduces the expression or activity of a polypeptide selected from the group consisting of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz, thereby treating the damaged or degenerating neuron.

9. The method of claim 8 further comprising contacting the damaged or degenerating neuron with another agent that reduces the expression or activity of an additional distinct polypeptide selected from the group consisting of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930, optionally to achieve a synergistic effect.

10. A method for increasing regeneration of a damaged or degenerating neuron in a subject in need thereof, the method comprising administering to the subject an agent that reduces the expression or activity of a polypeptide selected from the group consisting of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930, thereby increasing regeneration of the damaged or degenerating neuron in the subject.

11. The method of claim 8, wherein damage to the neuron is associated with an injury or a neurodegenerative disease.

12. The method of claim 8 further comprising contacting the damaged or degenerating neuron with another agent that reduces the expression or activity of an additional distinct polypeptide selected from the group consisting of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz, optionally to achieve a synergistic effect.

13. A pharmaceutical composition for increasing survival, or reducing death or degeneration of a damaged or degenerating neuron, the composition comprising: an agent that reduces the expression or activity of a polypeptide selected from the group consisting of ATF3, ATF4, ATF7IP, CEBPA, CEBPB, CEBPG, CEBPZ, CHOP, EBF3, ELMSAN1, Enoph1, Fastkd5, Fbp1, Fbp2, Fgfr2, Fgfr3, Flt1, Flt4, Frk, Gk5, Lats1, Lkb1, Lpin2, Lrp2, Ltk, MAP2K4, MAP2K7, Map3k11, Map3k19, MAPK8IP3, Nt5c1a, Pak4, Pask, Pdp1, Pdp2, Pgk1, Pi4kb, Pikfyve, Pklr, Pld2, Prkag2, Ptpn2, Ripk1, Ripk3, Rngtt, Sgpp1, Sh3kbp1, Slk, Smg1, Snrk, Speg, Sphk1, Sphk2, Srpk2, SSRP1, Stk10, Stk38l, SUPT16, Tesk1, Tie1, Tssk4, Uckl1, and Ywhaz and an excipient.

14. A pharmaceutical composition for increasing regeneration of a damaged or degenerating neuron, the composition comprising: an agent that reduces the expression or activity of a polypeptide selected from the group consisting of Bnc1, Carf, cdk9, Ctcf, Dido1, Ep300, Fastkd5, Foxq1, Lhx2, Lhx6, Pawr, Pax6, Phf5a, Rbbp7, Rnf141, Sertad1, Sim1, Sin3a, Snrk, Sox15, Sox7, Srf, Stk10, Stradα, Tcf24, Tcf3, Tgif1, Tial1, Tie1, Tmpo, and Zfp930 and a physiologically acceptable excipient.

15. The composition of claim 13, wherein the agent comprises a small molecule listed in Table 1, an inhibitory nucleic acid molecule comprising siRNA, shRNA, or antisense polynucleotide, or a CRISPR/Cas system.

16. A cell contacted with the pharmaceutical composition of claim 13.

17. A neuron produced by the method of claim 1.

18. A method of treating a neurodegenerative disease or nerve injury in a subject, the method comprising:
delivering to a subject in need thereof the cell of claim 16, wherein the cell is capable of differentiating into a neuron.

19. A method of treating a neurodegenerative disease or nerve injury in a subject, the method comprising:
delivering to a subject in need thereof the neuron of claim 17, wherein the neuron exhibits neuronal activity and function.

20. A kit comprising the agent of claim 13.

21. A cell or neuron suitable for implantation into a subject, wherein the cell or neuron has been contacted with the composition of claim 13.

22. A method for introducing a neuron into a subject, the method comprising administering the cell or neuron of claim 16 to the subject.


SEQUENCE LISTING (11 sequences)

SEQ ID NO: 1 - DNA, 5814 bp, Homo sapiens
  source 1..5814: mol_type = other DNA
agtgacgggcgcagcctcggcagcggcggcggcggagccctgaggcgacagcagctgcgggaggcgacgggctgcggcctgcggaacctgaggcagctggggagggccgggcgcgccggccggatagcgagccgcgctggcggcggcggtggccgcgatgatggagatccagatggacgagggcggcggcgtggtggtgtaccaggacgactactgctccggctcggtgatgtcggagcgggtgtcgggcctggcgggctccatctaccgcgagttcgagcgcctcatccactgctacgacgaggaggtggtcaaggagctcatgccgctggtggtgaacgtgctggagaacctagactcggtgctcagcgagaaccaggagcacgaggtggagctggagctgctgcgcgaggacaacgagcagctgctcacccagtacgagcgtgagaaggcgctgcgcaggcaggcggaggagaaattcattgagtttgaagatgctctggaacaagagaagaaagagctgcaaatccaggtggagcactacgagttccagacgcgccagctggagctgaaggccaagaactatgccgatcagatttcccggttggaggagcgggagtcggagatgaagaaggagtacaatgccctgcaccagcggcacacagagatgatacagacctacgtggagcacattgagaggtccaagatgcagcaggtcggaggaaacagccagaccgagagcagcctgccggggcggagcaggaaggagcgccccacctccctgaacgtgttccccctggctgacggcacggtacgtgcacagatcgggggcaagctcgtgcctgcgggggaccactggcacctgagtgacctcggccagctgcagtccagctccagctaccagtgtccacaggatgaaatgtccgagtcaggccagtcctcggcggccgccacacccagcaccacaggcaccaagtccaacacgcccacatcctccgtgccctcggccgccgtcacacccctcaacgagagcctgcagcccctgggggactatggcgtgggctccaagaacagcaagcgtgcccgggagaagcgcgacagccgcaacatggaagtacaggtcacccaggagatgcgcaacgtcagtataggcatgggcagcagtgacgagtggtctgatgttcaagacattattgactccacgccagagctggacatgtgtccagagacccgcctggaccgcacaggaagcagcccaacccagggcatcgtgaacaaagctttcggcatcaacaccgactccctgtaccatgagctgtcgacggcagggtctgaggtcatcggggatgtggacgaaggggccgacctcctaggggagttctcagtgcgcgatgatttctttggaatgggcaaagaagtggggaatctgctactggaaaactcacagcttctggaaaccaaaaacgccttgaatgtggtgaagaatgacctgattgccaaggtcgaccagctgtccggggagcaggaggtgctgaggggcgagttggaggctgctaagcaggccaaagtcaagctggaaaaccgtatcaaggagctggaagaggaactgaaaagagtgaagtccgaggccatcatcgcccgccgtgaacccaaagaagaggcggaggatgtaagcagctatctctgtacagaatcggacaaaatccccatggcccagcgccgccgcttcacgcgggtggagatggcccgtgtgctcatggagcggaaccagtacaaggagcggctgatggagctgcaggaggctgtgcggtggactgagatgatcagagcgtcccgagagcacccatccgtccaggagaagaagaagtcgaccatctggcagttcttcagccgcctcttcagctcttcctccagcccccctccggccaagcgcccctatccctcggtgaacatccactacaagtcacccaccactgccggcttcagccagcgccgcaaccatgccatgtgcccgatctcggcaggcagccggcccctggaattcttccctgacgacgactgcacgtcctccgcccgtcgagagcagaagcgcgagcagtaccgccaggtgcgtgagcacgtgcgtaacgacgacggccgtctgcaggcctgcggctggagcctgcccgccaagtacaagcagctgagtcccaacgggggccaggaggacacgcggatgaagaacgtgccggtgccggtgtactgccgccctctggtggagaaggaccccaccatgaagctgtggtgtgccgcgggcgtcaacctgagcgggtggaggcccaatgaggacgacgctgggaatggagtcaagccagcgccaggccgcgatcccctgacctgcgaccgcgaaggagacggcgagcccaagagcgcccacacgtctcccgagaagaagaaggccaaggagctccctgaaatggacgccacctccagccgggtgtggatcctgaccagcaccctgaccaccagcaaggtggtgatcatcgacgccaaccagccgggcacggtggtggaccagttcaccgtctgcaacgcgcacgtgctgtgcatctccagcatccccgcggccagcgacagcgactaccctcccggggagatgttcctggacagcgacgtgaacccagaggacccgggcgcagatggcgtgctggccggtatcaccctggtgggctgtgccacccgctgcaacgtgccgcggagcaactgctcctcccgaggggacaccccagtgctagacaaggggcagggggaggtggccaccatcgccaacgggaaggtcaacccgtcccagtccacagaggaggccacagaggccacggaggtgccagaccctgggcccagcgagccagagacagccacattgcggcccgggcctctcacagagcacgtcttcactgacccagccccgaccccgtcctctggcccccagcctggcagcgagaacgggccagagcctgacagcagcagcacacggccagagccagagcccagcggggaccccacgggagcaggcagcagtgctgcacccaccatgtggctgggagcccagaacggctggctctatgtgcactcggctgtggccaactggaagaagtgcctgcactccatcaagctgaaggattctgtgctgagcctggtgcatgtcaaaggccgtgtgctggtggctctggcggacgggaccctggccatcttccaccgtggtgaagatggccagtgggatctgagcaactatcacctaatggacctgggccacccgcaccactccatccgctgcatggctgttgtgtacgaccgcgtgtggtgtggctacaagaacaaggtgcacgtcatccagcccaagaccatgcagatagagaagtcatttgacgcccacccgcggcgggagagccaggtgcggcagctggcgtggatcggcgatggcgtatgggtgtccatccgcctggactccaccctgaggctctaccatgcacacacgcaccagcatctacaggacgtggacattgagccctacgtcagcaagatgctaggcactggcaagctgggtttctccttcgtacgcatcacggccctgcttgtcgcgggcagccggctctgggtgggcaccggcaacggagtggtcatctccatccccctgacagagagtgagtggcctgcacacctgcaggggcagtggtgctgccagaggtgtacgtgggttcacggggtggctctgcagggccaccttggagggtgccttgctgcccctacgctgaccgctctcctcttctcccatgctcctcccatgtccccagctgtggtcctgcaccgaggccagctcctggggctccgagccaataagacatcccccacctctggggagggcgcccgtcccgggggcatcatccacgtgtatggcgatgacagcagtgacagggcggccagcagcttcatcccctactgctccatggcccaggcccagctatgcttccatgggcaccgcgatgccgtgaagttctttgtctcggtgccagggaacgtgctggccaccctgaatgggagtgtgctggacagcccagccgagggccctgggccagctgcccctgcctcggaggtcgagggccagaagctgcggaacgtgctggtgctgagcggcggggagggctacatcgacttccgcattggagacggagaggacgacgagacggaggagggcgcaggggacatgagccaggtgaagcccgtgctgtccaaggcagagcgcagtcacatcatcgtgtggcaggtgtcctacacccccgagtgaagctgctgccctgcctggcccgacctgtacataggacccccgaccacctgacccccgcccggcccgcggggtagccagccaggcgccgccgcccctcttctaacctctcaacctgcagctttcacctgagtctggcccctccagcgggcagggagtgcggggatgcggatcagctgggaggaggaggggaggggaacttccacccgaggggaagatgctctcgggacagtttcccgggcagctcctggccagcttccagcccagagtcctcaagtccagggcaccttgggcccagcgcaggcagaatccgaggtggtcctggctctaccctgggcctcctactccccagcacccctggaggaggcaggggctccccgccgccgaggctgcctgccctgggcccacctctgcatgctgctcatggggccaccctgcctcctgggccctcactctgcctaggggagctgggccaggcactagcctttgcccagggaggtgggcctcaggctgcccaggtgcctgcaccccagccggccttctctggggcctccccgtcgtcaagcctctatcctgtctgtccccaccccagctgtcccctgcccagggagctggcataaaagcacgaggcccggctccctggggcagctgcttgagaacagagactgctaccccatcctgcccatgcaggcaggctcttgccagccccgttctgacccgtgtccccccaggctctgcctgggcagaagactcaccttggaggagtgggccctggagtcctgtccctcccagaagcccccagggtgggatttctcaggctgccagggcaggcccaggcctcaggaagaaggggaggcccctggcctctccgggatcagtcctaggacacaggctcagcctcaggttgatgggggatgatgtgctcccggggcctgcctcctgcacggggctccacggagcccagctcccagacacgctactaagtgcctagggttgcccgctgtggcctgcttccagggagcaacagagaggccaccaagcagaggcccgtggggctgaggatggagccgcccccagccgactccaagcccgcagagggcagacgccaccctggactgctctccctgcccagctgggcctctctggcctattcctaccttccaggcccactgcactcctgtctgggaggcccttatgagggcagcccagcccccgcacccacccccaaccagagaagcacagatcttggggagctgccccacaagccccgctggccaccgagggctgcagccgctgcgctgccggcttctccccaccaccctgccacctccactgtgatgtatgtccgctccctcgtctgttcccccaggatctcgaagtgactccgggctgagcagtggggcggctgggggaggggtgacgattctcctcaggctttggccctgcaagcaaacccacatatctgctctgtatgtaataaatgtcttaacgtcgta

SEQ ID NO: 2 - protein, 1336 aa, Homo sapiens
MMEIQMDEGGGVVVYQDDYCSGSVMSERVSGLAGSIYREFERLIHCYDEEVVKELMPLVVNVLENLDSVLSENQEHEVELELLREDNEQLLTQYEREKALRRQAEEKFIEFEDALEQEKKELQIQVEHYEFQTRQLELKAKNYADQISRLEERESEMKKEYNALHQRHTEMIQTYVEHIERSKMQQVGGNSQTESSLPGRRKERPTSLNVFPLADGTVRAQIGGKLVPAGDHWHLSDLGQLQSSSSYQCPQDEMSESGQSSAAATPSTTGTKSNTPTSSVPSAAVTPLNESLQPLGDYGVGSKNSKRAREKRDSRNMEVQVTQEMRNVSIGMGSSDEWSDVQDIIDSTPELDMCPETRLDRTGSSPTQGIVNKAFGINTDSLYHELSTAGSEVIGDVDEGADLLGEFSVRDDFFGMGKEVGNLLLENSQLLETKNALNVVKNDLIAKVDQLSGEQEVLRGELEAAKQAKVKLENRIKELEEELKRVKSEAIIARREPKEEAEDVSSYLCTESDKIPMAQRRRFTRVEMARVLMERNQYKERLMELQEAVRWTEMIRASREHPSVQEKKKSTIWQFFSRLFSSSSSPPPAKRPYPSVNIHYKSPTTAGFSQRRNHAMCPISAGSRPLEFFPDDDCTSSARREQKREQYRQVREHVRNDDGRLQACGWSLPAKYKQLSPNGGQEDTRMKNVPVPVYCRPLVEKDPTMKLWCAAGVNLSGWRPNEDDAGNGVKPAPGRDPLTCDREGDGEPKSAHTSPEKKKAKELPEMDATSSRVWILTSTLTTSKVVIIDANQPGTVVDQFTVCNAHVLCISSIPAASDSDYPPGEMFLDSDVNPEDPGADGVLAGITLVGCATRCNVPRSNCSSRGDTPVLDKGQGEVATIANGKVNPSQSTEEATEATEVPDPGPSEPETATLRPGPLTEHVFTDPAPTPSSGPQPGSENGPEPDSSSTRPEPEPSGDPTGAGSSAAPTMWLGAQNGWLYVHSAVANWKKCLHSIKLKDSVLSLVHVKGRVLVALADGTLAIFHRGEDGQWDLSNYHLMDLGHPHHSIRCMAVVYDRVWCGYKNKVHVIQPKTMQIEKSFDAHPRRESQVRQLAWIGDGVWVSIRLDSTLRLYHAHTHQHLQDVDIEPYVSKMLGTGKLGFSFVRITALLVAGSRLWVGTGNGVVISIPLTETVVLHRGQLLGLRANKTSPTSGEGARPGGIIHVYGDDSSDRAASSFIPYCSMAQAQLCFHGHRDAVKFFVSVPGNVLATLNGSVLDSPAEGPGPAAPASEVEGQKLRNVLVLSGGEGYIDFRIGDGEDDETEEGAGDMSQVKPVLSKAERSHIIVWQVSYTPE

SEQ ID NO: 3 - DNA, 1313 bp, Homo sapiens
  source 1..1313: mol_type = other DNA
gagcttgctgcattgcagccgccgcggcgccgctcggctcttcactcccaacaatggcggctccgagcccgagcggcggcggcggctccgggggcggcagcggcagcggcacccccggccccgtagggtccccggcgccaggccacccggccgtcagcagcatgcagggctttcagataaacttctgtgaaaaggcacaaagtaaacgcaaagcactgaagttgaattttgcaaatccacctttcaaatctacagcaaggtttactctgaatcccaatcctacaggagttcaaaacccacacatagagagactgagaacacacagcattgagtcatcaggaaaactgaagatctcccctgaacaacactgggatttcactgcagaggacttgaaagaccttggagaaattggacgaggagcttatggttctgtcaacaaaatggtccacaaaccaagtgggcaaataatggcagttaaaagaattcggtcaacagtggatgaaaaagaacaaaaacaacttcttatggatttggatgtagtaatgcggagtagtgattgcccatacattgttcagttttatggtgcactcttcagagagggtgactgttggatctgtatggaactcatgtctacctcgtttgataagttttacaaatatgtatatagtgtattagatgatgttattccagaagaaattttaggcaaaatcactttagcaactgtgaaagcactaaaccacttaaaagaaaacttgaaaattattcacagagatatcaaaccttccaatattcttctggacagaagtggaaatattaagctctgtgacttcggcatcagtggacagcttgtggactctattgccaagacaagagatgctggctgtaggccatacatggcacctgaaagaatagacccaagcgcatcacgacaaggatatgatgtccgctctgatgtctggagtttggggatcacattgataataggagtcaactattatctgcaactgcactaagaatgggaacaggacaagccagcttacctgcagtcaattcattgatgaaggcacataggcttctcccatctcaggcaaggtgtagtatgtccagtaatactgtatatagtgtgtatattaagtatacgtatttttatatgtggaaaatagtttctacttgcaatattctattcactgaatagtaaaatcacaagtgaatgagctgaaagtaattcagtttttgagggacaatatattagtccattctcacactactgtaaagaactacatgagactgggtaatttatgaagagaggttta

SEQ ID NO: 4 - protein, 399 aa, Homo sapiens
MAAPSPSGGGGSGGGSGSGTPGPVGSPAPGHPAVSSMQGKRKALKLNFANPPFKSTARFTLNPNPTGVQNPHIERLRTHSIESSGKLKISPEQHWDFTAEDLKDLGEIGRGAYGSVNKMVHKPSGQIMAVKRIRSTVDEKEQKQLLMDLDVVMRSSDCPYIVQFYGALFREGDCWICMELMSTSFDKFYKYVYSVLDDVIPEEILGKITLATVKALNHLKENLKIIHRDIKPSNILLDRSGNIKLCDFGISGQLVDSIAKTRDAGCRPYMAPERIDPSASRQGYDVRSDVWSLGITLYELATGRFPYPKWNSVFDQLTQVVKGDPPQLSNSEEREFSPSFINFVNLCLTKDESKRPKYKELLKHPFILMYEERAVEVACYVCKILDQMPATPSSPMYVD

SEQ ID NO: 5 - DNA, 3375 bp, Homo sapiens
  source 1..3375: mol_type = other DNA
agtgcggtgtttgtctgccggactgacgggcggccgggcggtgcgcggcggcggtggcggcggggaagatggcggcgtcctccctggaacagaagctgtcccgcctggaagcaaagctgaagcaggagaaccgggaggcccggcggaggatcgacctcaacctggatatcagcccccagcggcccaggcccaccctgcagctcccgctggccaacgatgggggcagccgctcgccatcctcagagagctccccgcagcaccccacgccccccgcccggccccgccacatgctggggctcccgtcaaccctgttcacaccccgcagcatggagagcattgagattgaccagaagctgcaggagatcatgaagcagacgggctacctgaccatcgggggccagcgctaccaggcagaaatcaacgacctggagaacttgggcgagatgggcagcggcacctgcggccaggtgtggaagatgcgcttccggaagaccggccacgtcattgccgttaagcaaatgcggcgctccgggaacaaggaggagaacaagcgcatcctcatggacctggatgtggtgctgaagagccacgactgcccctacatcgtgcagtgctttgggacgttcatcaccaacacggacgtcttcatcgccatggagctcatgggcacctgcgctgagaagctcaagaagcggatgcagggccccatccccgagcgcattctgggcaagatgacagtggcgattgtgaaggcgctgtactacctgaaggagaagcacggtgtcatccaccgcgacgtcaagccctccaacatcctgctggacgagcggggccagatcaagctctgcgacttcggcatcagcggccgcctggtggactccaaagccaagacgcggagcgccggctgtgccgcctacatggcacccgagcgcattgaccccccagaccccaccaagccggactatgacatccgggccgacgtatggagcctgggcatctcgttggtggagctggcaacaggacagtttccctacaagaactgcaagacggactttgaggtcctcaccaaagtcctacaggaagagcccccgcttctgcccggacacatgggcttctcgggggacttccagtccttcgtcaaagactgccttactaaagatcacaggaagagaccaaagtataataagctacttgaacacagcttcatcaagcgctacgagacgctggaggtggacgtggcgtcctggttcaaggatgtcatggcgaagactgagtcaccgcggactagcggcgtcctgagccagccccacctgcccttcttcaggtagctgcttggcggcggccagccccacagggggccaggggcatggccacaggcccccctccccacttggccacccagctgcctgccaggggagacctgggacctggacggccacctaggactgaggacagagagtggggggtgcccacccaccccccccgccccgggcctaccaagcccccgcccttcccaccccggggtcagccggccgtgtgcgtcccccgacagacactgtgaacggaagacagcaggccgcgatcagagtcgctgttcattcagccgcagcctctgggccggggcggcccccaggggccaggagagagccctggagtcccgcagccaccatgcacgctcccagcgtgctgtgtccttcgccactcccacgcgcccgttcctcttccgtcgccctctgtcccctgctctacctctctgtccttgtctggctctcccgtcaccctccctgcctctgtctctcttctggcctgagcctgggcccagccacctcctgacgggtcccctgggtctgcataggtctcccatggcgcaatgagtcagtggcccccagccaggcagtgtgggcattgccactgcggctggacggggctgcgcgctcgcgctctctctctctctctctctctctctttgatctcagggggtcctttttggagtttattgtattttattgtacttggtggggtgtttggggtgggggcggaggagagcttgttctcgtggggttgtcggtaccttcagaaacttttaccaaagtcacgattagctgcttgtggtggggccccaaccgccctcgggcactggggagctgggctggggctgctgctctggggtctccgggggccacagcttggggtgagttgaagacctcaggggatgtggaggggtctgcggggccctggccgcacaggatggccttcagggaaggtggtcttggggcatggtgcagagcaggtgaccggagggaatcggtgacggagcggggccaagggaggggtccggagggagtcagggatggagggcagagggagtggatgtgggggtttgaggacgtgtgacaagctccagcaggggtgggggccgggctgagggtgggggtgcgaggtggtcactcccatcgtgcccctggccgtccctccactcacccacacctggcccagtccacgttgaggtccaggactgggaaggaccgggtgagtgcaccggggacccaggccaggtgccccccggagcctgctggggtggccagagcaggagggggtgtgtttcctttttgtgggtgttgcatgcaaatcaagtggacaagaaaaaataacaaaacaaaaaacaagaaaaaaaaaacacaaaaccccgtaaaatcacaaagaaaatccaacaccaaaggcgcagaagccggctggccgtggtgggggcagcgtaggcgtagcatccctctcctctcacttagcctgttgactcttgttattatcatgatattcacaaaacgccgcatgtttaaaaagtcatagatgtcatcttctctctgcccccagggaggaaagccaccttctcttgccccttggcccctttgtcaggggccaggggtctgccgggtgggggtgccaacaggcctggccctttcctcccctgcatccagccatgggggcctctgcgattgccggaaggttgcatggctggtcccagggccagcacaggcccgaggccgggctgcctggttttatttttatttaactttattttctgttttatgagtgtgtgtccgcccacccccacccccttcagtgttaagtggggagccctgggggagtctctcctgcctcccagcctctcccaagacctcccccctcgtcaccagccatccctctggaccaggcagagggcggaccgggtgggcaggggcctgagggtggctcgggccagcccaccagccaatggacccctcctcaggccgccagtgtcgccctgcccctttttaaaacaaaatgccctcgtttgtaaacccttagacgcttgagaataaaccccttccttttcttcca

SEQ ID NO: 6 - protein, 419 aa, Homo sapiens
MAASSLEQKLSRLEAKLKQENREARRRIDLNLDISPQRPRPTLQLPLANDGGSRSPSSESSPQHPTPPARPRHMLGLPSTLFTPRSMESIEIDQKLQEIMKQTGYLTIGGQRYQAEINDLENLGEMGSGTCGQVWKMRFRKTGHVIAVKQMRRSGNKEENKRILMDLDVVLKSHDCPYIVQCFGTFITNTDVFIAMELMGTCAEKLKKRMQGPIPERILGKMTVAIVKALYYLKEKHGVIHRDVKPSNILLDERGQIKLCDFGISGRLVDSKAKTRSAGCAAYMAPERIDPPDPTKPDYDIRADVWSLGISLVELATGQFPYKNCKTDFEVLTKVLQEEPPLLPGHMGFSGDFQSFVKDCLTKDHRKRPKYNKLLEHSFIKRYETLEVDVASWFKDVMAKTESPRTSGVLSQPHLPFFR

SEQ ID NO: 7 - DNA, 3293 bp, Homo sapiens
  source 1..3293: mol_type = other DNA
gaggtaaacaagatggcggcggcgtgtcgggcgcggaagggggaggcggcccggggcgcccgcgagtgaggcgcggggcggcgaagggagcgcgggtggcggcacttgctgccgcggccttggatgggctgggcccccctcgccgctccgcctcctccacacgcgcggcggccgcggcgagggggacgcgccgcccggggcccggcaccttcgggaaccccccggcccggagcctgcggcctgcgccgcctcggccgccgggagccccgtggagcccccgccgccgcgccgccccgcggaccggacgctgagggcactcggggcggggcgcgcgctcgggcagacgtttgcggggaggggggcgcctgccgggccccggcgaccaccttgggggtcgcgggccggctcggggggcgcccagtgcgggccctcgcgggcgccgggcagcgaccagccctgagcggagctgttggccgcggcgggaggcctcccggacgcccccagccccccgaacgctcgcccgggccggcgggagtcggcgccccccgggaggtccgctcggtcgtccgcggcggagcgtttgctcctgggacaggcggtgggaccggggcgtcgccggagacgcccccagcgaagttgggctctccaggtgtgggggtcccggggggtagcgacgtcgcggacccggcctgtgggatgggcggcccggagaagactgcgctcggccgtgttcatacttgtccgtgggcctgaggtccccggaggatgacctagcactgaaaagccccggccggcctccccagggtccccgaggacgaagttgaccctgaccgggccgtctcccagttctgaggcccgggtcccactggaactcgcgtctgagccgccgtcccggacccccggtgcccgccggtccgcagaccctgcaccgggcttggactcgcagccgggactgacgtgtagaacaatcgtttctgttggaagaagggtttttcccttccttttggggtttttgttgccttttttttttcttttttctttgtaaaattttggagaagggaagtcggaacacaaggaaggaccgctcacccgcggactcagggctggcggcgggactccaggaccctgggtccagcatggaggtggtggacccgcagcagctgggcatgttcacggagggcgagctgatgtcggtgggtatggacacgttcatccaccgcatcgactccaccgaggtcatctaccagccgcgccgcaagcgggccaagctcatcggcaagtacctgatgggggacctgctgggggaaggctcttacggcaaggtgaaggaggtgctggactcggagacgctgtgcaggagggccgtcaagatcctcaagaagaagaagttgcgaaggatccccaacggggaggccaacgtgaagaaggaaattcaactactgaggaggttacggcacaaaaatgtcatccagctggtggatgtgttatacaacgaagagaagcagaaaatgtatatggtgatggagtactgcgtgtgtggcatgcaggaaatgctggacagcgtgccggagaagcgtttcccagtgtgccaggcccacgggtacttctgtcagctgattgacggcctggagtacctgcatagccagggcattgtgcacaaggacatcaagccggggaacctgctgctcaccaccggtggcaccctcaaaatctccgacctgggcgtggccgaggcactgcacccgttcgcggcggacgacacctgccggaccagccagggctccccggctttccagccgcccgagattgccaacggcctggacaccttctccggcttcaaggtggacatctggtcggctggggtcaccctctacaacatcaccacgggtctgtaccccttcgaaggggacaacatctacaagttgtttgagaacatcgggaaggggagctacgccatcccgggcgactgtggccccccgctctctgacctgctgaaagggatgcttgagtacgaaccggccaagaggttctccatccggcagatccggcagcacagctggttccggaagaaacatcctccggctgaagcaccagtgcccatcccaccgagcccagacaccaaggaccggtggcgcagcatgactgtggtgccgtacttggaggacctgcacggcgcggacgaggacgaggacctcttcgacatcgaggatgacatcatctacactcaggacttcacggtgcccggacaggtcccagaagaggaggccagtcacaatggacagcgccggggcctccccaaggccgtgtgtatgaacggcacagaggcggcgcagctgagcaccaaatccagggcggagggccgggcccccaaccctgcccgcaaggcctgctccgccagcagcaagatccgccggctgtcggcctgcaagcagcagtgaggctggccgcctgcagcccgtgtccaggagccccgccaggtgcccgcgccaggccctcagtcttcctgccggttccgcccgccctcccggagaggtggccgccatgcttctgtgccgaccacgccccaggacctccggagcgccctgcagggccgggcagggggacagcagggaccgggcgcagccctcccccctcggccgcccggcagtgcacgcggcttgttgacttcgcagccccgggcggagccttcccgggcgggcgtgggaggagggaggcggcctccatgcactttatgtggagactactggccccgcccgtggcctcgtgctccgcagggcgcccagcgccgtccggcggccccgccgcagaccagctggcgggtgtggagaccaggctcctgaccccgccatgcatgcagcgccacctggaagccgcgcggccgctttggttttttgtttggttggttccattttctttttttctttttttttttaagaaaaaataaaaggtggatttgagctgtggctgtgaggggtgtttgggagctgctgggtggcaggggggctgtggggtcgggctcacgtcgcggccgcctttgcgctctcgggtcaccctgctttggcggcccggccggagggcaggaccctcacctctcccccaaggccactgcgctcttgggaccccagagaaaacccggagcaagcaggagtgtgcggtcaatatttatatcatccagaaaagaaaaacacgagaaacgccatcgcgggatggtgcagacgcggcggggactcggagggtgccgtgcgggcgaggccgcccaaatttggcaataaataaagcttgggaagcttgga

SEQ ID NO: 8 - protein, 433 aa, Homo sapiens
MEVVDPQQLGMFTEGELMSVGMDTFIHRIDSTEVIYQPRRKRAKLIGKYLMGDLLGEGSYGKVKEVLDSETLCRRAVKILKKKKLRRIPNGEANVKKEIQLLRRLRHKNVIQLVDVLYNEEKQKMYMVMEYCVCGMQEMLDSVPEKRFPVCQAHGYFCQLIDGLEYLHSQGIVHKDIKPGNLLLTTGGTLKISDLGVAEALHPFAADDTCRTSQGSPAFQPPEIANGLDTFSGFKVDIWSAGVTLYNITTGLYPFEGDNIYKLFENIGKGSYAIPGDCGPPLSDLLKGMLEYEPAKRFSIRQIRQHSWFRKKHPPAEAPVPIPPSPDTKDRWRSMTVVPYLEDLHGADEDEDLFDIEDDIIYTQDFTVPGQVPEEEASHNGQRRGLPKAVCMNGTEAAQLSTKSRAEGRAPNPARKACSASSKIRRLSACKQQ

SEQ ID NO: 9 - DNA, 5126 bp, Homo sapiens
  source 1..5126: mol_type = other DNA
agctacccggcaccccctccccgcggccggcagcccgctcggtattatgattagcgctgggtgcggggtttcggcggccgggagggagttgtcggcgccgcggccgctgcggacggacgctcgcctgccggctgagatatccatgacgacattgaaaatgaattttttgtattcaccagatattcttatatgagaagatctattttaaacagtctaaatattttttcttctgttggaccagcatggcaggatttaagcgagggtatgatggaaagattgctggattatatgatctggataaaaccttgggtcgaggccattttgccgtggttaaacttgccaggcatgtctttacgggtgaaaaggtggcagtaaaagttattgacaagacaaaactggacactctagctactggtcatcttttccaggaagtgagatgcatgaaactagtgcagcatcctaacatcgtccgcctttatgaagttattgacacccagaccaaactatatcttattctagaacttggggatggaggagatatgtttgattatataatgaaacatgaggagggtcttaatgaagacttggccaagaagtattttgctcagatagttcatgctatatcttattgccataaactccatgtggttcacagagacttaaaaccagagaatgtagtcttctttgaaaaacaaggtcttgtaaagttgacagactttgggttcagcaacaaatttcaaccagggaagaagctcactacaagctgtggatctcttgcatattccgctccagaaattctgcttggtgatgagtatgatgcacctgcagtagatatttggagtctgggagtgatccttttcatgttggtgtgtgggcagccgccctttcaagaagccaatgacagtgaaacactgacaatgatcatggattgcaaatatacagtaccatcccatgtgtctaaagagtgtaaagacctaatcacacggatgctacagagagatcccaagagaagggcttctttagaagagattgaaaatcatccttggcttcagggagtggacccttcaccagctacaaagtataacattccccttgtgtcatacaaaaatctctcggaagaggagcacaacagcatcattcagcgcatggtgcttggggacatagcggatcgagacgccattgtagaagccctggaaaccaacaggtataaccatatcacagccacatacttcctgctggctgaaaggatcctgagagaaaagcaagagaaagaaatacagaccagatctgcaagcccgagcaatatcaaggcccagtttaggcagtcatggccaaccaaaattgatgtaccccaggaccttgaggatgacctcacggccactcctttgtcccacgcgactgtccctcagtctcctgctcgggctgctgacagtgtcctcaatggccacaggagcaaaggcctgtgtgactcagctaagaaagatgacctccctgagttggctggaccagcactctctacggtgccacccgcaagcttaaaacccacagccagtgggcggaagtgtctgttcagggtggaagaagatgaagaggaagatgaggaggacaagaaacccatgtccctctcaacacaagtggttttgcgccggaagccatctgtaaccaaccgcctgacatccaggaagagtgcgcccgtcctcaaccagatctttgaggaaggggaatctgacgatgagtttgacatggatgagaatctgcctcccaagttgagcaggttaaagatgaatatagcttctccaggtacagttcacaaacgctaccaccggaggaaaagtcagggccggggctccagctgcagtagttcggagaccagtgatgatgattctgaaagccggcggcggctcgataaagatagcgggttcacctactcctggcaccgacgggatagcagcgaggggccccctggcagtgagggggatggcgggggccagagcaagccaagcaatgccagtggaggggtggacaaggccagccccagtgagaacaatgctggtgggggcagtccctccagcggctcgggtggcaaccccaccaatacatcgggtaccacacgccgctgtgccggccccagcaactccatgcagctggcctctcgcagtgctggggagctcgttgagagcctcaaactcatgagcctctgcctcggctcccagcttcatgggagcaccaagtacattattgatccacagaatggcttgtcattttccagtgtgaaagtccaagagaaatctacgtggaaaatgtgcattagctccacagggaatgcagggcaggtccctgcagtgggcggcataaagtttttctctgaccacatggcagataccaccactgaattggaacggataaagagcaagaacctgaaaaataacgtgctgcagctacctctgtgcgaaaagaccatctctgtgaacatccagcggaaccctaaggaggggctgctgtgcgcatccagcccagccagctgttgccatgtcatctgactgtggccccatctggccgctagcacgcttcctgctcagagcagtgaagaccggctcacttcactgttccatttggttttactattttaaagtgggcgttaggagcaattatttattacctttccatttgttcgcctgatgatgtgacaatgcatggtctttgtgcatgctgctagacacttttctttcccagccgaaaagcctattatgtaatttttacattcataattttaatgtggatgatcaggattaaatcaagatatatatctggaacctcttataaatggagcacttagaaatttgttgttctgcacttaacctagagagagaaaaaatgcttttctttgtgaaaaatctgaattcctgtcctgaccttctgtgatgtggaaaccctaggctctgagacacactctctggtgtctgagacagaaccaaagcaataacgttgtgatgcccacaggcctggagccagctagcgaccttgtgccgcccagctgtccatggcccgtgcagagcagaggacagtgagtgtctgcactgagaaccttaaaccacagttgaacatacccacacctgtttgtcttaagctatagtgtaaaaacaaagtttgggctctgaaaatttaactgaaaaagatttccttgtttttgtaataggtgagataaagtacttagatttataaggcagcttcccctgtagtgataaattacaagcagacaatcttattttgtaatgtgatgaagtgatgatgtcttaactctacttagagagtgtatgtctgtctaacagaacaaaaagatgctctgtgtaaattccttcctgtagggcacactgcaggatttccatgtagatagaagaactatagggcctagtacagaaggtgcacacaaatgttggcaaagtcaaaaccccatgaattaaaacctactggaatttggtttttaggagtttggtaattagattatctcttttgttattttcattcagttatatcctttggctcagctagctttgaaattggctgatgaaaaaatatacataaaagggtaaaattcacacatacagcaaacaaaaatgcacaaagcctgcttcgtaacttttttttctggaattgtttttcactttgcctttttctgccaaaacaataatcaaagaactcttgctttaacctattcctgtacaaagactgtttttgaccagataatcatctgttgtggcattctatcttgtaggacactgtatattgcaaattgctgattatggaaggggccagttgctgttttttcatgcagtgccctgggagtcttaaaagcagtgcttagcaacattggtgatagcatgtggctgggacccagggcccttccccactcttcagccccgagtcatgtgtctgaggtgacggactgagacgcatctggtcctgtaattcagagagtgggcacatcaccaaagaactgcattgctgtggtcactgtttcttcaagtacacactgactctgctactttaggataaatatattttactcagaactctgaatttcacagtatacttactaaactaagtaaaaatgatacttaaaatacttattttactttctagacctaggctagatgttttaagctacagctctagttcattgtgatatttataatttgaaagctatgagaatagatgtgtgggtgaagccatagaacatatttgcttgaaattcttgagcagggatcttataaagggccagaaataagatgtgtggttcacatagatagtgagcgtaacatctgtattaaacataggagagaagtttataaagggcattggcaataaactctttgttgcagctgttttccaagcagtgtaaatactttttcctgtgattatgtatagccttggaatggcaccttttaactaacccatatgtgtttggtttcaatggttttttatattcagatgtatatatggtgctcactttaggatcagcagtgttgaccatttatgctgcatagctgtattatagccttattagttgtgtggttgacccttggggtatacaaatgtcagtctgagtggtgtcttactcctttgtttataagtgaatgattgtgcatgttttgtatgtcatagtatgtcgtcacataaaagggagggagcgaaaaaccattacattaagataatattggaccaaactacttacttgctctaaacagttacttgtaccccttaacctgtcttcaaaagttgcatatagttacagtagtgtataaattaaatattgtggaaaaacagtcttgtatttttctgtatgtgtgtatatatatataattatgtacttctggcaattctatctgtatttaaagatgtgacaatcttgacaccaattttaagaatagctgtgagaccgaattaaagataatccctaccaagtgaaaattgatgtgtgttaagagggtacagaattatcaactgatttggtcagttgcttccaatgctggttgatttccctcattgtgtaaacattgacaggtatgtgacaaatgggaaaaaaaatccaaataataaagtgacatattggtgttcagcaatataaa

SEQ ID NO: 10 - protein, 765 aa, Homo sapiens
MAGFKRGYDGKIAGLYDLDKTLGRGHFAVVKLARHVFTGEKVAVKVIDKTKLDTLATGHLFQEVRCMKLVQHPNIVRLYEVIDTQTKLYLILELGDGGDMFDYIMKHEEGLNEDLAKKYFAQIVHAISYCHKLHVVHRDLKPENVVFFEKQGLVKLTDFGFSNKFQPGKKLTTSCGSLAYSAPEILLGDEYDAPAVDIWSLGVILFMLVCGQPPFQEANDSETLTMIMDCKYTVPSHVSKECKDLITRMLQRDPKRRASLEEIENHPWLQGVDPSPATKYNIPLVSYKNLSEEEHNSIIQRMVLGDIADRDAIVEALETNRYNHITATYFLLAERILREKQEKEIQTRSASPSNIKAQFRQSWPTKIDVPQDLEDDLTATPLSHATVPQSPARAADSVLNGHRSKGLCDSAKKDDLPELAGPALSTVPPASLKPTASGRKCLFRVEEDEEEDEEDKKPMSLSTQVVLRRKPSVTNRLTSRKSAPVLNQIFEEGESDDEFDMDENLPPKLSRLKMNIASPGTVHKRYHRRKSQGRGSSCSSSETSDDDSESRRRLDKDSGFTYSWHRRDSSEGPPGSEGDGGGQSKPSNASGGVDKASPSENNAGGGSPSSGSGGNPTNTSGTTRRCAGPSNSMQLASRSAGELVESLKLMSLCLGSQLHGSTKYIIDPQNGLSFSSVKVQEKSTWKMCISSTGNAGQVPAVGGIKFFSDHMADTTTELERIKSKNLKNNVLQLPLCEKTISVNIQRNPKEGLLCASSPASCCHVI

SEQ ID NO: 11 - protein, 10 aa, Adeno-associated virus
LADQDYTKTA